(19) United States
(12) Patent Application Publication
(10) Pub. No.: US 2023/0383261 A1
(43) Pub. Date: Nov. 30, 2023

(54) HEPATOCYTE-LIKE CELLS

(71) Applicant: Sana Biotechnology, Inc., Seattle, WA (US)

(72) Inventors: John Michael Rukstalis, Milton, MA (US); Ryan M. Hasselkus, Quincy, MA (US); Elizabeth Suzanne Howe Vera, Melrose, MA (US)

(21) Appl. No.: 18/031,771

(22) PCT Filed: Oct. 13, 2021
(86) PCT No.: PCT/US21/54853

§ 371 Date: Apr. 13, 2023

Related U.S. Application Data
(60) Provisional application No. 63/152,791, filed on Feb. 23, 2021.
(60) Provisional application No. 63/091,477, filed on Oct. 14, 2020.

(51) Int. Cl. C12N 5/071

(52) U.S. Cl. CPC C12N 5/067; C12N 2510/00; C12N 2506/45; C12N 2501/01; C12N 2533/50; C12N 2513/00; C12N 2500/02; C12N 2500/38

(57) ABSTRACT

Provided herein are hepatocyte-like cells with enhanced in vitro ureagenesis capability and methods for producing and using such cells. The subject hepatocyte-like cells are produced by differentiating a source cell into a hepatocyte-like cell in the presence of one or more ureagenesis enhancer. The subject hepatocyte-like cells provided are useful for the treatment of liver disorders, particularly those where hepatocyte ureagenesis function is impaired.

### CROSS-REFERENCE TO RELATED APPLICATION

This application claims priority under 35 U.S.C. § 119(e) to U.S. Provisional Application Nos. 63/091,477 filed Oct. 14, 2020 and 63/152,791 filed Feb. 23, 2021, the disclosures of which are herein incorporated by reference in their entireties.

### BACKGROUND

The liver provides a wide range of crucial bodily functions including metabolism and neutralization of toxins. Despite its ability to regenerate, the liver cannot sustain injuries beyond a certain threshold.

Ammonia, a toxic waste metabolite, is constant produced from amino acid catabolism. Ammonia is resolved in the liver, where the urea cycle coverts free ammonia to urea via ureagenesis. Liver malfunctions such as acute liver disease, can lead to decreased ureagenesis and hyperammonemia. Untreated, the high amounts of ammonia associated with these liver disorders can cause brain damage, coma and eventually death.

Currently, liver transplants provide an effective long-term treatment for liver failure. Liver transplantation, however, is an expensive invasive surgical procedure that is limited by a shortage of healthy liver donors. Hepatocyte transplantation is an alternative to liver transplantation in certain liver disorders. Such transplantation involves a relatively less complicated surgical procedure that can be repeated several times if unsuccessful. Shortage of donor hepatocytes, however, limits the use of such methods.

Functional hepatocyte-like cells generated from multiple cell types (e.g., stem cells and fibroblasts) offer a possible solution to the shortage of donor hepatocytes. Hepatocyte-like cells differentiated using existing in vitro methods, however, exhibit significantly low levels of urea production as compared to primary hepatocyte counterparts. Moreover, these technologies often rely on spontaneous improvement in ureagenesis after in vivo implantation. Therefore, treatments using such cells may require high dosage levels to provide sufficient urea production. Such cells may also exhibit ureagenesis variability, depending on the presence of in vivo ureagenesis enhancers in the patient.

Thus, there remains a need for novel approaches, compositions and methods for the treatment of liver disorders, particularly those that associated with decreased ureagenesis.

### BRIEF SUMMARY

Provided herein are hepatocyte-like cells with enhanced in vitro ureagenesis capability and methods for producing and using such cells. The subject hepatocyte-like cells are produced by differentiating a source cell into a mature hepatocyte-like cell in the presence of one or more ureagenesis enhancers.

The subject hepatocyte-like cells provided are useful for the treatment of liver disorders, particularly those where hepatocyte ureagenesis function is impaired. In some embodiments, such hepatocyte-like cells advantageously allow for a reduction in total cell dose required for the treatments of disorders with decreased ureagenesis. Further, as the subject hepatocyte-like cells exhibit enhanced ureagenesis in vitro, it is possible to determine urea production of such cells prior to treatment, thereby reducing the batch-to-batch ureagenesis variability associated with cells generated using previous methods.

In one aspect, provided herein is an isolated hepatocyte-like cell that exhibits enhanced ureagenesis capability. In some embodiments, the cell produces converts ammonia at a rate of at least 3 nmol/min/10⁶ cells following stimulation with 5 mM or 10 mM ammonia.

In certain embodiments, the cell has increased expression of one or more urea cycle pathway enzymes. In exemplary embodiments, the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments, the hepatocyte-like cell has increased RNA expression of the one or more urea cycle pathway enzymes. In certain embodiments, the cell has increased protein expression of the one or more urea cycle pathway enzymes. In certain embodiments, the cell has increased expression of one or more genes selected from the group consisting of albumin (ALB), asialoglycoprotein receptor 1 (ASGR1), ASGR2, alpha fetoprotein (AFP), glucose-6-phosphatase catalytic subunit (G6PC), hepatocyte nuclear factor 4 alpha (HNF4a), keratin, type I cytoskeletal 18 (KRT18), SOX9, SERPINA1, CYP1A1, CYP1A2, CYP2C19, CYP2B6, CYP2D6, CYP3A4, and CYP3A7. In some embodiments, the cell secretes one or more of albumin, α-1 antitrypsin (A1AT), and coagulation Factor V.

In some embodiments, the cell has cytochrome p450 activity. In exemplary embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In certain embodiments, the cell has glycogen synthesis capability and/or storage capability. In some embodiments, the cell has low density lipoprotein (LDL) uptake and/or storage capability. In exemplary embodiments, the cell has lipid storage capability. In some embodiments, the cell has indocyanine green (ICG) uptake and/or clearance capability. In certain embodiments, the cell has gamma-glutamyl transpeptidase activity.

In another aspect, provided herein is a composition that includes a population of any of the isolated hepatocyte-like cells described herein. In some embodiments, the composition further includes a pharmaceutically acceptable carrier.

In one aspect, provided herein is a composition that includes: a) a polymer matrix; and b) a population of any of the hepatocyte-like cells described herein. In some embodiments, the population of the hepatocyte-like cells is encapsulated by the polymer matrix. In certain embodiments, the polymer matrix is semipermeable. In some embodiments, the polymer matrix includes alginate, a modified alginate, chitosan, hydroxyethyl methacrylate, methyl methacrylate, agarose, collagen, polylysine, polyethersulfone, polysulfone, polyornithine, and/or aminopropylsilicate or combinations thereof.

In another aspect, provided herein is a method that includes the steps of: a) providing a source cell; b) differentiating the source cell in vitro in at least one culture medium that includes an agent that increases intracellular cyclic AMP, wherein the source cell differentiates into a mature hepatocyte-like cell having enhanced in vitro ureagenesis capability; and recovering the mature hepatocyte-like cell.

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) takes places in a two-dimensional (2D) culture system. In certain embodiments, the two-dimensional culture system includes a substrate that includes an extracellular matrix (ECM) component. In some embodiments, the ECM component includes laminin and/or collagen. In some embodiments, the substrate is fetal bovine serum (FBS) free. In some embodiments, the two-dimensional culture system includes a soft hydrogel substrate. In some embodiments, the soft hydrogel substrate has an elastic modulus ranging from about 1 kPa to about 8 kPa. In certain embodiments, the soft hydrogel includes poly(ethylene glycol) (PEG). In some embodiments, the ECM component includes an ECM maturation component.

In some embodiments of this method, the differentiating step b) takes places in a three-dimensional culture system. In certain embodiments, the three-dimensional culture system includes an inverse colloidal crystal scaffold. In some embodiments, the inverse colloidal crystal scaffold is coated with an extracellular matrix (ECM) component. In particular embodiments, the extracellular matrix (ECM) component includes laminin and/or collagen.

In some embodiments of this method, the differentiating step b) is carried out in the presence of an endothelial cell. In particular embodiments, the endothelial cell is a human umbilical vein endothelial cell (HUVEC).

In some embodiments of this method, the differentiating step b) is carried out at one or more oxygen conditions. In some embodiments, the one or more oxygen conditions comprise a hypoxic condition. In some embodiments, the one or more oxygen conditions comprise a normoxic condition. In some embodiments of this method, the differentiating step b) is carried out at increased oxygen conditions. In some embodiments, the mature hepatocyte-like cell is contacted with vitamin K selected from the group consisting of vitamin K1, vitamin K2, and both vitamins K1 and K2. In some embodiments of this method, the mature hepatocyte-like cell is contacted with vitamin K1.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydroxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In one aspect, provided herein is a method for producing a mature hepatocyte-like cell having enhanced ureagenesis capability. This method includes the steps of: a) providing a source cell; b) differentiating the source cell in vitro in a two dimensional culture system that includes a fetal bovine serum (FBS) free substrate that includes an extracellular matrix (ECM) component; and at least one culture medium that includes an agent that increases intracellular cyclic AMP, wherein the source cell differentiates into a mature hepatocyte-like cell having enhanced in vitro ureagenesis capability; and c) recovering the mature hepatocyte-like cell. In exemplary embodiments, the extracellular matrix component includes laminin and/or collagen.

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) takes places in a two-dimensional (2D) culture system. In certain embodiments, the two-dimensional culture system includes a substrate that includes an extracellular matrix (ECM) component. In some embodiments, the two-dimensional culture system includes a soft hydrogel substrate. In certain embodiments, the soft hydrogel includes poly(ethylene glycol) (PEG). In some embodiments, the soft hydrogel substrate has an elastic modulus ranging from about 1 kPa to about 8 kPa. In some embodiments, the ECM component includes an ECM maturation component.

In some embodiments of this method, the differentiating step b) takes places in a three-dimensional culture system. In certain embodiments, the three-dimensional culture system includes an inverse colloidal crystal scaffold. In some embodiments, the inverse colloidal crystal scaffold is coated with an extracellular matrix (ECM) component. In particular embodiments, the extracellular matrix (ECM) component includes laminin and/or collagen.

In some embodiments of this method, the differentiating step b) is carried out in the presence of an endothelial cell. In particular embodiments, the endothelial cell is a human umbilical vein endothelial cell (HUVEC).

In some embodiments of this method, the differentiating step b) is carried out at one or more oxygen conditions. In some embodiments, the one or more oxygen conditions comprise a hypoxic condition. In some embodiments, the one or more oxygen conditions comprise a normoxic condition. In some embodiments of this method, the differentiating step b) is carried out at increased oxygen conditions. In some embodiments, the mature hepatocyte-like cell is contacted with vitamin K selected from the group consisting of vitamin K1, vitamin K2, and both vitamins K1 and K2. In some embodiments of this method, the mature hepatocyte-like cell is contacted with vitamin K1.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydoxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In another aspect, provided herein is a method for producing a mature hepatocyte-like cell having enhanced ureagenesis capability. This method includes the steps of: a) providing a source cell; b) differentiating the source cell in vitro in a two dimensional culture system that includes: i. a soft hydrogel substrate; and ii. at least one culture medium that includes an agent that increases intracellular cyclic AMP, wherein the source cell differentiates into a mature hepatocyte-like cell having enhanced in vitro ureagenesis capability; and c) recovering the mature hepatocyte-like cell. In some embodiments, the soft hydrogel substrate has an elastic modulus ranging from about 1 kPa to about 8 kPa. In some embodiments, the soft hydrogel has an elastic modulus of less than 5 kPa. In certain embodiments, the soft hydrogel includes poly(ethylene glycol) (PEG).

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) is carried out in the presence of an endothelial cell. In particular embodiments, the endothelial cell is a human umbilical vein endothelial cell (HUVEC).

In some embodiments of this method, the differentiating step b) is carried out at one or more oxygen conditions. In some embodiments, the one or more oxygen conditions comprise a hypoxic condition. In some embodiments, the one or more oxygen conditions comprise a normoxic condition. In some embodiments of this method, the differentiating step b) is carried out at increased oxygen conditions. In some embodiments, the mature hepatocyte-like cell is contacted with vitamin K selected from the group consisting of vitamin K1, vitamin K2, and both vitamins K1 and K2. In some embodiments of this method, the mature hepatocyte-like cell is contacted with vitamin K1.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydoxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In one aspect, provided herein is a method for producing a mature hepatocyte-like cell having enhanced ureagenesis capability. This method includes the steps of: a) providing a source cell; b) differentiating the source cell in vitro in at least one culture medium that includes an agent that increases intracellular cyclic AMP, wherein the source cell differentiates into a mature hepatocyte-like cell having enhanced in vitro ureagenesis capability; and c) recovering the mature hepatocyte-like cell. In this method, the differentiating is initially carried out on a first substrate that includes gelatin and fetal bovine serum and transferred to a second substrate that includes laminin and/or collagen on about day 14 of the differentiating. In exemplary embodiments, the second substrate further includes fetal bovine serum.

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) is carried out in the presence of an endothelial cell. In particular embodiments, the endothelial cell is a human umbilical vein endothelial cell (HUVEC).

In some embodiments of this method, the differentiating step b) is carried out at one or more oxygen conditions. In some embodiments, the one or more oxygen conditions comprise a hypoxic condition. In some embodiments, the one or more oxygen conditions comprise a normoxic condition. In some embodiments of this method, the differentiating step b) is carried out at increased oxygen conditions. In some embodiments, the mature hepatocyte-like cell is contacted with vitamin K selected from the group consisting of vitamin K1, vitamin K2, and both vitamins K1 and K2. In some embodiments of this method, the mature hepatocyte-like cell is contacted with vitamin K1.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydoxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In another aspect, provided herein is a method for producing a mature hepatocyte-like cell having enhanced ureagenesis capability. This method includes the steps of: a) providing a source cell; b) differentiating the source cell in vitro in normoxia condition in at least one culture medium that includes an agent that increases intracellular cyclic AMP, wherein the source cell differentiates into a mature hepatocyte-like cell having enhanced in vitro ureagenesis capability; and c) recovering the mature hepatocyte-like cell. In certain embodiments, the normoxia condition is ˜20% partial pressure of 02.

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) is carried out in the presence of an endothelial cell. In particular embodiments, the endothelial cell is a human umbilical vein endothelial cell (HUVEC).

In some embodiments of this method, the mature hepatocyte-like cell is contacted with vitamin K1.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydoxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In another aspect, provided herein is a method for producing a mature hepatocyte-like cell having enhanced ureagenesis capability. This method includes the steps of: a) providing a source cell; b) differentiating the source cell in vitro in at least one culture medium that includes vitamin K1, wherein the source cell differentiates into a mature hepatocyte-like cell having enhanced in vitro ureagenesis capability; and c) recovering the mature hepatocyte-like cell. In some embodiments, the vitamin K1 is at a concentration of 750 μM-10 mM.

In some embodiments of this method, the source cell is a stem cell, a fibroblast, a gastric epithelial cell, a ductal cell, or a hepatocyte. In exemplary embodiments, the source cell is a stem cell. In certain embodiments, stem cell is an induced pluripotent stem cell.

In some embodiments of this method, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In exemplary embodiments, the agent is forskolin. In some embodiments, the culture medium includes 5-20 M of forskolin.

In some embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments of this method, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments of this method, the source cell differentiates into an immature hepatocyte-like cell prior to differentiating into the mature hepatocyte-like cell. In exemplary embodiments, the differentiating step b) takes place during the differentiation of the immature hepatocyte-like cell to the mature hepatocyte-like cell.

In some embodiments of this method, the differentiating step b) is carried out in the presence of an endothelial cell. In particular embodiments, the endothelial cell is a human umbilical vein endothelial cell (HUVEC).

In some embodiments of this method, the differentiating step b) is carried out at increased oxygen conditions.

In some embodiments of this method, the method further includes step d) encapsulating the mature hepatocyte-like cell in a polymer matrix. In exemplary embodiments, the polymer matrix is semipermeable. In some embodiments, the polymer matrix includes alginate, a modified alginate, chitosan, hydoxyethyl methacrylate, methyl methacrylate, agarose, collagen, polylysine, polyethersulfone, polysulfone, polyornithine, and/or aminopropylsilicate or combinations thereof.

In some embodiments of this method, the mature hepatocyte-like cell has increased RNA expression of the one or more urea cycle pathway enzymes. In exemplary embodiments, the mature hepatocyte-like cell has increased protein expression of the one or more urea cycle pathway enzymes.

In some embodiments of this method, culture medium further includes hepatocyte growth factor (HGF), and oncostatin-M (OSM). In certain embodiments, the culture medium is free of hepatocyte growth factor (HGF).

In some embodiments of this method, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

In certain embodiments of this method, the mature hepatocyte-like cell secretes albumin and/or α-1 antitrypsin (A1AT).

In some embodiments of this method, the mature hepatocyte-like cell has cytochrome p450 activity. In particular embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments of this method, the mature hepatocyte-like cell has glycogen synthesis capability and/or storage capability. In certain embodiments, the mature hepatocyte-like cell has low density lipoprotein (LDL) uptake and/or storage capability. In some embodiments of this method, the mature hepatocyte-like cell has lipid storage capability. In exemplary embodiments, the mature hepatocyte-like cell has indocyanine green (ICG) uptake and/or clearance capability. In particular embodiments, the mature hepatocyte-like cell has gamma-glutamyl transpeptidase activity.

In another aspect, provided herein is a method for treating a liver disorder in a patient that includes administering to the patient a therapeutically effective amount of a population of hepatocyte-like cells that includes enhanced ureagenesis capability.

In some embodiments, the population of hepatocyte-like cells convert ammonia at a rate of at least 3 nmol/min/10⁶ cells following stimulation with 5 mM or 10 mM ammonia. In certain embodiments, the hepatocyte-like cells further have increased expression of one or more urea cycle pathway enzymes. In exemplary embodiments, the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments, the hepatocyte-like cells have increased RNA expression of the one or more urea cycle pathway enzymes. In certain embodiments, the hepatocyte-like cells have increased protein expression of the one or more urea cycle pathway enzymes.

In some embodiments, the liver disorder is selected from the group consisting of fibrosis, cirrhosis, end-stage liver disease, a metabolic liver disease, acute liver failure, and chronic liver failure.

In exemplary embodiments, the administering includes grafting the population of hepatocyte-like cells into the patient's liver. In certain embodiments, the grafting includes injecting the population of hepatocyte-like cells into the patient. In some embodiments, the injecting of the population of hepatocyte-like cells is into the patient's liver. In some embodiments, the hepatocyte-like cells have increased expression of one or more genes selected from the group consisting of albumin (ALB), asialoglycoprotein receptor 1 (ASGR1), ASGR2, alpha fetoprotein (AFP), glucose-6-phosphatase catalytic subunit (G6PC), hepatocyte nuclear factor 4 alpha (HNF4a), keratin, type I cytoskeletal 18 (KRT18), SOX9, SERPINA1, CYP1A1, CYP1A2, CYP2C19, CYP2B6, CYP2D6, CYP3A4, and CYP3A7. In some embodiments, the hepatocyte-like cells secrete albumin and/or α-1 antitrypsin (A1AT).

In certain embodiments, the hepatocyte-like cells have cytochrome p450 activity. In certain embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments, the hepatocyte-like cells have glycogen synthesis capability and/or storage capability. In certain embodiments, the hepatocyte-like cells have low density lipoprotein (LDL) uptake and/or storage capability. In some embodiments, the hepatocyte-like cells have lipid storage capability. In certain embodiments, the hepatocyte-like cells have indocyanine green (ICG) uptake and/or clearance capability. In some embodiments, the hepatocyte-like cells have gamma-glutamyl transpeptidase activity.

In yet another aspect, provided herein is a mature hepatocyte-like cell differentiated from a source cell, wherein the mature hepatocyte-like cell has increased ureagenesis capability, wherein the source cell is differentiated in vitro in at least one culture medium that includes an agent that increases intracellular cyclic AMP. In some embodiments, the source cell is a stem cell, a fibroblast, a gastric epithelial cell, a ductal cell, or a hepatocyte. In certain embodiments, the source cell is a stem cell. In exemplary embodiments, the stem cell is an induced pluripotent stem cell.

In some embodiments, the agent is selected from the group consisting of forskolin, glucagon, glucagon-like peptide-1 (GLP-1), glucose-dependent insulinotropic peptide (GIP), and an analog thereof. In exemplary embodiments, the agent is forskolin. In some embodiments, the culture medium includes 5-20 μM of forskolin.

In certain embodiments, the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments, the mature hepatocyte-like cell has increased RNA expression of the one or more urea cycle pathway enzymes. In certain embodiments, the mature hepatocyte-like cell has increased protein expression of the one or more urea cycle pathway enzymes.

In some embodiments, the source cell differentiates into a progenitor hepatocyte-like cell prior to differentiating into the mature hepatocyte-like cell. In certain embodiments, the differentiating step b) takes place after the source cell differentiates into the progenitor hepatocyte-like cell.

In some embodiments, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In exemplary embodiments, the mature hepatocyte-like cell secretes albumin and/or α-1 antitrypsin (A1AT).

In some embodiments, the mature hepatocyte-like cell has cytochrome p450 activity. In certain embodiments, the cytochrome p450 activity includes activity of one or more cytochrome p450 family members selected from the group consisting of CYP1A1, CYP1A2, CYP2C19, CYP2B6, CYP2D6, CYP3A4, and CYP3A7.

In some embodiments, the mature hepatocyte-like cell has glycogen synthesis capability and/or storage capability. In some embodiments, the mature hepatocyte-like cell has low density lipoprotein (LDL) uptake and/or storage capability. In certain embodiments, the mature hepatocyte-like cell has lipid storage capability. In exemplary embodiments, the mature hepatocyte-like cell has indocyanine green (ICG) uptake and/or clearance capability. In some embodiments, the mature hepatocyte-like cell has gamma-glutamyl transpeptidase activity.

In one aspect, provided herein is a composition that includes: a) a polymer matrix; and b) a population of any of the mature hepatocyte-like cells described herein, wherein the population of the isolated hepatocyte-like cell is encapsulated by the polymer matrix. In certain embodiments, the polymer matrix is semipermeable. In some embodiments, the polymer matrix includes alginate, a modified alginate, chitosan, hydroxyethyl methacrylate, methyl methacrylate, agarose, collagen, polylysine, polyethersulfone, polysulfone, polyornithine, and/or aminopropylsilicate or combinations thereof.

In another aspect, provided herein is a method that includes the steps of: a) providing a mature hepatocyte-like cell that has been differentiated in vitro from a source cell that is not a hepatocyte or hepatocyte like cell; b) culturing the mature hepatocyte-like cell in at least one culture medium that includes an agent that increases intracellular cyclic AMP; and c) recovering the mature hepatocyte-like cell. In some embodiments, the source cell is a stem cell, a fibroblast, a gastric epithelial cell, or a ductal cell. In certain embodiments, the source cell is a stem cell. In some embodiments, the stem cell is an induced pluripotent stem cell.

In exemplary embodiments, the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing. In some embodiments, the agent is forskolin. In particular embodiments, the culture medium includes 5-20 μM of forskolin.

In another aspect, provided herein is a hepatocyte-like cell that exhibits enhanced ureagenesis capability for the treatment of a liver disorder. In some embodiments, the cell converts ammonia at a rate of at least 3 nmol/min/10⁶ cells following stimulation with 5 mM or 10 mM ammonia.

In certain embodiments, the cell further has increased expression of one or more urea cycle pathway enzymes. In exemplary embodiments, the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments, the cell has increased RNA expression of the one or more urea cycle pathway enzymes. In certain embodiments, the cell has increased protein expression of the one or more urea cycle pathway enzymes.

In exemplary embodiments, the liver disorder is selected from the group consisting of fibrosis, cirrhosis, end-stage liver disease, a metabolic liver disease, acute liver failure, and chronic liver failure.

In some embodiments, the cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In some embodiments, the cell secretes albumin and/or α-1 antitrypsin (A1AT).

In certain embodiments, the cell has cytochrome p450 activity. In exemplary embodiments, the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

In some embodiments, the cell has glycogen synthesis capability and/or storage capability. In exemplary embodiments, the cell has low density lipoprotein (LDL) uptake and/or storage capability. In certain embodiments, the cell has lipid storage capability.

In some embodiments, the cell has indocyanine green (ICG) uptake and/or clearance capability.

In yet another aspect, provided herein is the use of a hepatocyte-like cell comprising enhanced ureagenesis capability in the manufacture of a medicament for the treatment of a liver disorder in a patient in need thereof. In some embodiments, the cell converts ammonia at a rate of at least 3 nmol/min/10⁶ cells following stimulation with 5 mM or 10 mM ammonia.

In certain embodiments, the cell further has increased expression of one or more urea cycle pathway enzymes. In exemplary embodiments, the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

In some embodiments, the cell has increased RNA expression of the one or more urea cycle pathway enzymes. In certain embodiments, the cell has increased protein expression of the one or more urea cycle pathway enzymes.

In exemplary embodiments, the liver disorder is selected from the group consisting of fibrosis, cirrhosis, end-stage liver disease, a metabolic liver disease, acute liver failure, and chronic liver failure.

In some embodiments, the cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In some embodiments, the cell secretes albumin and/or α-1 antitrypsin (A1AT).

In certain embodiments, the cell has cytochrome p450 activity. In exemplary embodiments, the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

In some embodiments, the cell has glycogen synthesis capability and/or storage capability. In exemplary embodiments, the cell has low density lipoprotein (LDL) uptake and/or storage capability. In certain embodiments, the cell has lipid storage capability.

In some embodiments, the cell has indocyanine green (ICG) uptake and/or clearance capability. In certain embodiments, the cell has gamma-glutamyl transpeptidase activity.

In another aspect, provided herein is a method for producing a mature hepatocyte-like cell having enhanced ureagenesis capability comprising: (a) providing a source cell; (b) differentiating the source cell in vitro in a three dimensional culture system comprising at least one culture medium comprising an agent that increases intracellular cyclic AMP, wherein the source cell differentiates into a mature hepatocyte-like cell having enhanced in vitro ureagenesis capability; and (c) recovering the mature hepatocyte-like cell.

In some embodiments, the three dimensional culture system further comprises a soft hydrogel substrate. In some embodiments, the soft hydrogel substrate has an elastic modulus ranging from about 1 kPa to about 8 kPa. In some embodiments, the soft hydrogel substrate comprises poly(ethylene glycol) (PEG).

In some embodiments, the three-dimensional culture system comprises an inverse colloidal crystal scaffold. In some embodiments, the inverse colloidal crystal scaffold is coated with an extracellular matrix (ECM) component. In some embodiments, the extracellular matrix (ECM) component comprises laminin and/or collagen.

In some embodiments, the three dimensional culture system further comprises a polymer matrix. In some embodiments, the polymer matrix is semipermeable. In some embodiments, the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

In some embodiments, the source cell is selected from the group consisting of a stem cell, a fibroblast, a gastric epithelial cell, and a ductal cell. In some embodiments, the source cell is a stem cell. In some embodiments, the stem cell is an induced pluripotent stem cell.

In some embodiments, the agent is selected from the group consisting of forskolin, glucagon, glucagon-like peptide-1 (GLP-1), glucose-dependent insulinotropic peptide (GIP), and an analog thereof. In some embodiments, the agent is forskolin. In some embodiments, the culture medium comprises 5-20 μM of forskolin.

In some embodiments, the culture medium further comprises hepatocyte growth factor (HGF) and oncostatin-M (OSM). In some embodiments, the culture medium is free of hepatocyte growth factor (HGF).

In some embodiments, the differentiating step b) is carried out at one or more oxygen conditions. In some embodiments, the one or more oxygen conditions comprise a hypoxic condition. In some embodiments, the one or more oxygen conditions comprise a normoxic condition.

In some embodiments, the mature hepatocyte-like cell is contacted with vitamin K selected from the group consisting of vitamin K1, vitamin K2, and both vitamins K1 and K2.

In some embodiments, the mature hepatocyte-like cell further has increased expression of one or more urea cycle pathway enzymes. In some embodiments, the one or more urea cycle pathway enzymes are selected from the group consisting of forskolin carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In some embodiments, the mature hepatocyte-like cell has increased RNA expression of the one or more urea cycle pathway enzymes. In some embodiments, the mature hepatocyte-like cell has increased protein expression of the one or more urea cycle pathway enzymes.

In some embodiments, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In some embodiments, the mature hepatocyte-like cell secretes albumin and/or α-1 antitrypsin (A1AT).

In some embodiments, the mature hepatocyte-like cell has cytochrome p450 activity. In some embodiments, the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

In some embodiments, the mature hepatocyte-like cell has glycogen synthesis capability and/or storage capability. In some embodiments, the mature hepatocyte-like cell has low density lipoprotein (LDL) uptake and/or storage capability. In some embodiments, the mature hepatocyte-like cell has lipid storage capability. In some embodiments, the mature hepatocyte-like cell has indocyanine green (ICG) uptake and/or clearance capability. In certain embodiments, the cell has gamma-glutamyl transpeptidase activity

In another aspect, provided herein is a method for producing a mature hepatocyte-like cell having enhanced ureagenesis capability comprising: (a) providing a source cell; (b) differentiating the source cell in vitro in a three dimensional culture system comprising: i) a fetal bovine serum (FBS) free substrate; and ii) at least one culture medium comprising an agent that increases intracellular cyclic AMP, wherein the source cell differentiates into a mature hepatocyte-like cell having enhanced in vitro ureagenesis capability; and (c) recovering the mature hepatocyte-like cell.

In some embodiments, the FBS free substrate comprises a soft hydrogel substrate. In some embodiments, the soft hydrogel substrate has an elastic modulus ranging from about 1 kPa to about 8 kPa. In some embodiments, the soft hydrogel substrate comprises poly(ethylene glycol) (PEG).

In some embodiments, the three-dimensional culture system comprises an inverse colloidal crystal scaffold. In some embodiments, the inverse colloidal crystal scaffold is coated with an extracellular matrix (ECM) component. In some embodiments, the extracellular matrix (ECM) component comprises laminin and/or collagen.

In some embodiments, the three dimensional culture system further comprises a polymer matrix. In some embodiments, the polymer matrix is semipermeable. In some embodiments, the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

In some embodiments, the source cell is selected from the group consisting of a stem cell, a fibroblast, a gastric epithelial cell, and a ductal cell. In some embodiments, the source cell is a stem cell. In some embodiments, the stem cell is an induced pluripotent stem cell.

In some embodiments, the agent is selected from the group consisting of forskolin, glucagon, glucagon-like peptide-1 (GLP-1), glucose-dependent insulinotropic peptide (GIP), and an analog thereof. In some embodiments, the agent is forskolin. In some embodiments, the culture medium comprises 5-20 μM of forskolin.

In some embodiments, the culture medium further comprises hepatocyte growth factor (HGF) and oncostatin-M (OSM). In some embodiments, the culture medium is free of hepatocyte growth factor (HGF).

In some embodiments, the differentiating step b) is carried out at one or more oxygen conditions. In some embodiments, the one or more oxygen conditions comprise a hypoxic condition. In some embodiments, the one or more oxygen conditions comprise a normoxic condition.

In some embodiments, the mature hepatocyte-like cell is contacted with vitamin K selected from the group consisting of vitamin K1, vitamin K2, and both vitamins K1 and K2.

In some embodiments, the mature hepatocyte-like cell further has increased expression of one or more urea cycle pathway enzymes. In some embodiments, the one or more urea cycle pathway enzymes are selected from the group consisting of forskolin carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In some embodiments, the mature hepatocyte-like cell has increased RNA expression of the one or more urea cycle pathway enzymes. In some embodiments, the mature hepatocyte-like cell has increased protein expression of the one or more urea cycle pathway enzymes.

In some embodiments, the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7. In some embodiments, the mature hepatocyte-like cell secretes albumin and/or α-1 antitrypsin (A1AT).

In some embodiments, the mature hepatocyte-like cell has cytochrome p450 activity. In some embodiments, the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

In some embodiments, the mature hepatocyte-like cell has glycogen synthesis capability and/or storage capability. In some embodiments, the mature hepatocyte-like cell has low density lipoprotein (LDL) uptake and/or storage capability. In some embodiments, the mature hepatocyte-like cell has lipid storage capability. In some embodiments, the mature hepatocyte-like cell has indocyanine green (ICG) uptake and/or clearance capability. In certain embodiments, the cell has gamma-glutamyl transpeptidase activity

### BRIEF DESCRIPTION OF THE DRAWINGS

FIGS. 1A-1B are line graphs depicting relative quantification (RQ) of CPS1 (FIG. 1A) and ARG1 (FIG. 1B) at days 0, 14, 21, 28 and 35 of differentiation from iPSC to hepatocyte-like cells. Cells were cultured + or −10 mM forskolin from days 28-35. RQ of primary human hepatocytes (PHH) is also included for reference.

FIGS. 2A-2B are images of fluorescent immunoblots of expression of CPS1 (FIG. 2A) and ARG1 (FIG. 2B) at day 35 of differentiation from iPSC to hepatocyte-like cells. Cells were cultured + or −10 mM forskolin from days 28-35. Immunoblot of primary human hepatocytes (PHH) is also included for reference.

FIG. 3A shows a diagram of a 3D differentiation protocol. FIG. 3B shows a diagram of a 3D differentiation protocol that includes the addition of forskolin during maturation of hepatocytes.

FIG. 4A shows percentages of CD184 (CXCR) positive cells produced using 3D differentiation processes in accordance with the present technology (FIG. 3B). FIG. 4B shows percentages of CD117 (c-kit) positive cells produced using the 3D differentiation processes.

FIGS. 5A and 5B are phase contrast images of suspension aggregation cultures from the 3D differentiation process of the present technology. FIG. 5C shows HNFa expression at day 0, day 14 and day 21 of the differentiation process using three different pluripotent stem cell lines. FIG. 5D shows albumin expression at day 0, day 14 and day 21 of the differentiation process using three different pluripotent stem cell lines. FIG. 5E is phase contrast image of the aggregated spheroids.

FIG. 6 are fluorescent immunostaining images of HNF4a and albumin expression in aggregated spheroids at day 21 of differentiation (see, for example, FIG. 3B and detailed 3D suspension protocols outlined below).

FIGS. 7A and 7B show gene expression analysis of hepatocyte maturation genes (FIG. 7A) and urea cycle genes (FIG. 7B) in hepatocyte-like cells generated using a 3D differentiation protocol (FIG. 3A) or a 3D differentiation protocol with the addition of 10 μM forskolin from days 28 to day 35 (FIG. 3B).

FIGS. 8A and 8B show the albumin production rate of hepatocyte-like cells differentiated from two iPSC cell lines and cultured on either a soft PEG hydrogel or a stiff PEG hydrogel.

FIGS. 9A-9C show the urea synthesis of hepatocyte-like cells that were produced upon treatment with either vitamin K1, vitamin K2 MK4, vitamin K2 MK7, or vitamin E. Urea synthesis was analysed using basal media (FIG. 9A), media containing ammonia (FIG. 9B), and media containing ammonia and arginine.

FIG. 10 shows CYP3A4 activity in mature hepatocyte-like cells upon treatment with media containing HGF and media lacking HGF.

FIG. 11 shows urea production in hepatocyte-like cells upon the addition of either 5 μM or 10 μM forskolin during hepatocyte maturation.

FIG. 12 shows urea production in hepatocyte-like cells that were cultured in either a hypoxia or normoxia condition and differentiated using either a 2D adherent method and 3D suspension method for differentiating and/or maturing cells into hepatocyte-like cells.

FIG. 13A shows a schematic diagram of a 3D differentiation method for differentiating and/or maturing cells into hepatocyte-like cells and followed by alginate encapsulation of such cells. FIG. 13B shows phase contrast images of aggregates formed from different amounts of cells. FIG. 13C shows that aggregates containing about 750 cells yielded clusters of significant size with negligible necrosis.

FIG. 14A shows a schematic diagram of various differentiation methods that include forming: (1) alginate encapsulated spheroids, (2) naked spheroids, (3) alginate encapsulated single-cell suspensions, or (4) 2D cell layers. FIG. 14B shows albumin production in hepatocyte-like cells produced using the methods of FIG. 14A. FIG. 14C shows basal urea secretion in these hepatocyte-like cells.

FIGS. 15A-15C show phase contrast and immunofluorescent images of cells in the alginate encapsulated spheroids, naked spheroids and alginate encapsulated single-cell suspension. The immunostaining detected live cells. FIG. 15A shows the cells 7 days post encapsulation or a corresponding time for the naked clusters. FIG. 15B shows the cells 3 days post encapsulation or a corresponding time for the naked clusters. FIG. 15C shows the cells 5 days post encapsulation or a corresponding time for the naked clusters.

FIGS. 16A-16B show urea production in hepatocytes cultured using a 2D differentiation method.

FIG. 17 shows urea production in hepatocytes cultured using a 3D differentiation method including alginate encapsulation.

FIG. 18 shows heat maps and hierarchal clustering of urea cycle gene in the samples from human fetal hepatocytes, day 35 hepatocytes differentiated using a 3D differentiation method, day 6 and day 35 hepatocytes differentiated using a 2D differentiation method, and primary human hepatocytes (PHH).

FIG. 19 shows images of alginate encapsulated hepatocyte spheroids and encapsulated single-cell suspensions.

FIG. 20 shows a schematic diagram of transplantation of encapsulated in vitro differentiated hepatocytes into a mouse model of acute liver failure. Ex vivo ureagenesis analysis was performed on the transplanted cells.

FIGS. 21A-21D show ex vivo urea production in the hepatocytes exposed to basal media and increasing amounts of ammonia. In particular, FIG. 21A shows the activity of the encapsulated mature hepatocytes. FIG. 21B shows the activity of the encapsulated mature hepatocytes and non-parenchymal cells. FIG. 21C shows the activity of murine primary hepatocytes. FIG. 21D shows the activity of empty beads.

FIGS. 22A-22E show AFP expression in hepatocytes differentiating from pluripotent stem cells at various days of the differentiation method A (FIGS. 22A and 22C), the differentiation method B (FIG. 22B) and the differentiation method C (FIGS. 22D and 22E). FIGS. 22A-22E, 23A-23E, 24A-24E, 25A-25E, 26A-26E, 27A-27E, 28A-28E, 29A-29D, 30A-30D, 31A-31D, 32A-32D, 33A-33D, 34A-34D, and 35A-35D show gene expression data for hepatocytes differentiated using one of the following methods: 3D differentiation method with forskolin treatment at days 21-35 (method A), a 3D differentiation method with forskolin treatment at days 30-35 (method B), and a 2D differentiation method with forskolin treatment at days 28-35 (method C).

FIGS. 23A-23E show ALB expression in hepatocytes differentiating from pluripotent stem cells at various days of the differentiation method A (FIGS. 23A and 23C), the differentiation method B (FIG. 23B), and the differentiation method C (FIGS. 23D and 23E).

FIGS. 24A-24E show ARG1 expression in hepatocytes differentiating from pluripotent stem cells at various days of the differentiation method A (FIGS. 24A and 24C), the differentiation method B (FIG. 24B), and the differentiation method C (FIGS. 24D and 24E).

FIGS. 25A-25E show ASGR1 expression in hepatocytes differentiating from pluripotent stem cells at various days of the differentiation method A (FIGS. 25A and 25C), the differentiation method B (FIG. 25B) and the differentiation method C (FIGS. 25D and 25E).

FIGS. 26A-26E show ASL1 expression in hepatocytes differentiating from pluripotent stem cells at various days of the differentiation method A (FIGS. 26A and 26C), the differentiation method B (FIG. 26B), and the differentiation method C (FIGS. 26D and 26E).

FIGS. 27A-27E show ASS1 expression in hepatocytes differentiating from pluripotent stem cells at various days of the differentiation method A (FIGS. 27A and 27C), the differentiation method B (FIG. 27B), and the differentiation method C (FIGS. 27D and 27E).

FIGS. 28A-28E show CPS1 expression in hepatocytes differentiating from pluripotent stem cells at various days of the differentiation method A (FIGS. 28A and 28C), the differentiation method B (FIG. 28B), and the differentiation method C (FIGS. 28D and 28E).

FIGS. 29A-29D show G6PC expression in hepatocytes differentiating from pluripotent stem cells at various days of the differentiation method A (FIGS. 29A and 29C), the differentiation method B (FIG. 29B), and the differentiation method C (FIG. 29D).

FIGS. 30A-30D show HNFa expression in hepatocytes differentiating from pluripotent stem cells at various days of the differentiation method A (FIGS. 30A and 30C), the differentiation method B (FIG. 30B), and the differentiation method C (FIG. 30D).

FIGS. 31A-31D show KRT18 expression in hepatocytes differentiating from pluripotent stem cells at various days of the differentiation method A (FIGS. 31A and 31C), the differentiation method B (FIG. 31B), and the differentiation method C (FIG. 31D).

FIGS. 32A-32D show NAGS expression in hepatocytes differentiating from pluripotent stem cells at various days of the differentiation method A (FIGS. 32A and 32C), the differentiation method B (FIG. 32B), and the differentiation method C (FIG. 32D).

FIGS. 33A-33C show NKX2.5 expression in hepatocytes differentiating from pluripotent stem cells at various days of the differentiation method A (FIGS. 33A and 33C) and the differentiation method B (FIG. 33B). TNNT2 expression in hepatocytes differentiating from pluripotent stem cells at various days using the differentiation C is shown in FIG. 33D.

FIGS. 34A-34D show OTC expression in hepatocytes differentiating from pluripotent stem cells at various days of the differentiation method A (FIGS. 34A and 34C), the differentiation method B (FIG. 34B), and the differentiation method C (FIG. 34D).

FIGS. 35A-35D show SOX9 expression in hepatocytes differentiating from pluripotent stem cells at various days of the differentiation method A (FIGS. 35A and 35C), the differentiation method B (FIG. 35B) and the differentiation method C (FIG. 35D).

Other objects, advantages and embodiments of the present technology will be apparent from the detailed description following.

### DETAILED DESCRIPTION

### I. Introduction

Hepatocyte-like cells differentiated using existing in vitro methods exhibit significantly low levels of urea production as compared to primary hepatocyte counterparts. See, e.g., Yu et al., Stem Cell Res. 9(3): 196-207 (2012). Therefore, treatments using such cells may require high dosage levels to provide sufficient urea production. These technologies also often rely on spontaneous improvement in ureagenesis after in vivo implantation. Thus, cells using these methods exhibiting varying levels of urea production, depending on the presence of in vivo ureagenesis enhancers in the patient.

In contrast, hepatocyte-like cells differentiated using the methods provided herein exhibit enhanced ureagenesis capability in vitro. In some embodiments, such hepatocyte-like cells advantageously allow for a reduction in total cell dose required for the treatments of disorders with decreased ureagenesis. Further, as the subject hepatocyte-like cells exhibit enhanced ureagenesis in vitro, it is possible to assess urea production of such cells prior to treatment, thereby reducing batch-to-batch ureagenesis variability associated with previous methods. Aspects of the subject hepatocyte-like cells and methods for producing and using such cells are discussed in further detail below.

### II. Definitions

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

As used herein, the terms “grafting”, “administering,” “introducing”, “implanting” and “transplanting” as well as grammatical variations thereof are used interchangeably in the context of the placement of cells (e.g., cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the technology. Any recited method may be carried out in the order of events recited or in any other order that is logically possible. Although any methods and materials similar or equivalent to those described herein may also be used in the practice or testing of the technology, representative illustrative methods and materials are now described.

Before the technology is further described, it is to be understood that this technology is not limited to particular embodiments described, as such may, of course, vary. It is also to be understood that the terminology used herein is for describing particular embodiments only, and is not intended to be limiting, since the scope of the present technology will be limited only by the appended claims.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the technology. The upper and lower limits of these smaller ranges may independently be included in the smaller ranges and are encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

### III. Detailed Description of the Embodiments

A. Mature Hepatocyte-Like Cells

In one aspect, provided herein are hepatocyte-like cells with enhanced ureagenesis capability. Ureagenesis refers to the process by which a cell converts ammonia to produce urea using the urea cycle. In particular embodiments, the hepatocyte-like cells develop and exhibit enhanced ureagenesis in vitro. In contrast to previous methods that rely on variable in vivo processes to develop hepatocyte-like cells with improved ureagenesis, the cells provided herein exhibit enhanced ureagenesis in vitro as a result of an in vitro differentiation process in the presence of one more ureagenesis enhancers. One of skill in the art can assess enhanced ureagenesis of the subject hepatocyte-like cells prior to transplantation, thereby advantageously reducing potential batch-to-batch variability associated with hepatocyte-like cells using existing methods.

As used herein, the term “enhanced ureagenesis capability” refers an improved ability to convert ammonia to urea by the urea cycle as compared to a reference. Ureagenesis can be measured using any suitable assay known in the art, for example, colorimetric urea assays (e.g., QuantiChrom Assay Kit by BiosAssay Systems). In some embodiments, enhanced ureagenesis capability is compared to a reference cell. In particular embodiments, the reference cell is a mature hepatocyte-like cell differentiated in the absence of one or more of the ureagenesis enhancers provided herein. In some embodiments, enhanced ureagenesis capability of the mature hepatocyte-like cell differentiated in the presence of a particular ureagenesis enhancer is at least 10%, 15%, 20%, 25%, 30%, 35%, 40%, 45%, 50%, 55%, 60%, 65%, 70%, 75%, 85%, 90%, 95% or 99% more ureagenesis as compared to a reference mature hepatocyte-like cell differentiated in the absence of the ureagenesis enhancer. In exemplary embodiments, the subject mature hepatocyte-like cell provided herein exhibits ureagenesis at a level comparable to a wild type mature hepatocyte. In exemplary embodiments, the subject the mature hepatocyte-like cell provided herein exhibits at least 50%, 55%, 60%, 65%, 70%, 75%, 85%, 90%, 95% or 99% ureagenesis as compared to a reference wild-type mature hepatocyte.

In some embodiments, the hepatocyte-like cell exhibits stimulation of ureagenesis activity when stimulated with ammonia. For example, the hepatocyte-like cell exhibits a ureagenesis rate of at least 3, 4, 5, 6, 8, 10, 12, 15, 20, or 25 nmol/min/10⁶ cells following stimulation with 5 mM or 10 mM ammonia. In some embodiments, the hepatocyte like cell exhibits a ureagenesis rate following stimulation with 5 mM or 10 mM ammonia of between 3 and 50 nmol/min/10⁶ cells, between 3 and 40 nmol/min/10⁶ cells, between 3 and 30 nmol/min/10⁶ cells, between 4 and 50 nmol/min/10⁶ cells, between 4 and 40 nmol/min/10⁶ cells, between 4 and 30 nmol/min/10⁶ cells, between 5 and 50 nmol/min/10⁶ cells, between 5 and 40 nmol/min/10⁶ cells, between 5 and 30 nmol/min/10⁶ cells, between 6 and 50 nmol/min/10⁶ cells, between 6 and 40 nmol/min/10⁶ cells, between 6 and 30 nmol/min/10⁶ cells, between 8 and 50 nmol/min/10⁶ cells, between 8 and 40 nmol/min/10⁶ cells, between 8 and 30 nmol/min/10⁶ cells, between 10 and 50 nmol/min/10⁶ cells, between 10 and 40 nmol/min/10⁶ cells, between 10 and 30 nmol/min/10⁶ cells, between 12 and 50 nmol/min/10⁶ cells, between 12 and 40 nmol/min/10⁶ cells, between 12 and 30 nmol/min/10⁶ cells, between 15 and 50 nmol/min/10⁶ cells, between 15 and 40 nmol/min/10⁶ cells, between 15 and 30 nmol/min/10⁶ cells, between 20 and 50 nmol/min/10⁶ cells, between 20 and 40 nmol/min/10⁶ cells, or between 20 and 30 nmol/min/10⁶ cells.

In particular embodiments, the hepatocyte-like cell with enhanced ureagenesis capability exhibits increased expression of one or more urea cycle pathway enzymes. Exemplary urea cycle pathway enzymes include, but are not limited to, carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In particular embodiments, the hepatocyte-like cell exhibits expression of one of the following urea cycle enzymes: CPS1, NAGS, ARG1, ASL, ASS1, or OTC. In some embodiments, the hepatocyte-like cell exhibits increased RNA transcript expression of one or more urea cycle pathway enzymes. In exemplary embodiments, the hepatocyte-like cell exhibits increased protein expression of one or more urea cycle pathway enzymes. In some embodiments, the mature hepatocyte-like cell exhibits increased protein expression of one or more urea cycle pathway enzymes at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In some embodiments, the subject mature hepatocyte-like cell exhibits a similar expression level of one or more urea cycle pathway enzymes as compared to a reference wild-type mature hepatocyte. In exemplary embodiments, the subject mature hepatocyte-like cell exhibits at least 50%, 55%, 60%, 65%, 70%, 75%, 85%, 90%, 95% or 99% the level of expression of one or more urea pathway enzymes as compared to a reference wild-type mature hepatocyte.

As used herein, a “mature hepatocyte-like cell” refers to a cell that exhibits one or more characteristics of a mature hepatocyte including, but not limited to: albumin secretion, α-1 antitrypsin (A1AT) secretion, cytochrome p450 activity, glycogen synthesis capability and/or storage capability, lipid (e.g., low density lipoprotein (LDL)) uptake and/or storage capability, indocyanine green (ICG) uptake and/or clearance capability, and gamma-glutamyl transpeptide activity. In exemplary embodiments, the mature hepatocyte-like cells provided herein exhibit enhanced ureagenesis capability and at least one or more of other characteristics of a mature hepatocyte disclosed herein. In exemplary embodiments, the subject mature hepatocyte-like cell exhibits enhanced ureagenesis capability and 2, 3, 4, 5, 6, 7, 8, 9 or 10 additional characteristics of a mature hepatocyte disclosed herein.

In some embodiments, the mature hepatocyte-like cell exhibits albumin secretion. In some embodiments, the mature hepatocyte-like cell secretes albumin at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of albumin secretion of a reference wild-type mature hepatocyte (e.g., a primary human hepatocyte). Albumin secretion can be assessed using any suitable technique include, for example, enzyme-linked immunosorbent (ELISA) assays and immunohistochemistry techniques (see, e.g., Wu et al., Cell Stem Cell 14: 394-403 (2014), which are incorporated herein by reference, particularly in parts pertinent to albumin secretion assessment). In certain embodiments, the mature hepatocyte-like cell exhibits increased expression of the albumin gene (ALB).

In some embodiments, the mature hepatocyte-like cell exhibits α-1 antitrypsin (A1AT) secretion. In some embodiments, the mature hepatocyte-like cell secretes A1AT at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of AAT secretion of a reference wild-type mature hepatocyte (e.g., a primary human hepatocyte). A1AT secretion can be assessed using any suitable technique include, for example, enzyme-linked immunosorbent (ELISA) assays and immunohistochemistry techniques (see, e.g., Wu et al., Cell Stem Cell 14: 394-403 (2014), which is incorporated herein by reference, particularly in parts pertinent to A1AT secretion assessment). In certain embodiments, the mature hepatocyte-like cell exhibits increased expression of the α-1 antitrypsin gene (SERPINA1). In some embodiments, the mature hepatocyte-like cell expresses the SERPINA1 gene at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell expresses the SERPINA1 gene at least 50%, 55%, 60%, 65%, 70%, 75%, 85%, 90%, 95% or 99% the level of AAT secretion of a reference wild-type mature hepatocyte (e.g., a primary human hepatocyte).

In some embodiments, the mature hepatocyte-like cell exhibits coagulation Factor V secretion. In some embodiments, the mature hepatocyte-like cell secretes Factor V at an amount that is at least 10%, 15%0, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of Factor V secretion of a reference wild-type mature hepatocyte (e.g., a primary human hepatocyte). Factor V secretion can be assessed using any suitable technique include, for example, enzyme-linked immunosorbent (ELISA) assays and immunohistochemistry techniques. In certain embodiments, the mature hepatocyte-like cell exhibits increased expression of the coagulation Factor V gene (F5).

In exemplary embodiments, the mature-hepatocyte-like cell exhibits glycogen synthesis capability and/or storage capability. In some embodiments, the mature hepatocyte-like cell exhibits at least 10%, 15%, 20%, 25%, 30%, 35%, 40%, 45%, 50%, 55%, 60%, 65%, 70%, 75%, 85%, 90%, 95% or 99% more glycogen synthesis capability and/or storage capability than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level glycogen synthesis capability and/or storage capability of a reference wild-type mature hepatocyte (e.g., a primary human hepatocyte). Glycogen synthesis capability and/or storage capability can be assessed, for example, using immunohistochemistry techniques (see, e.g., Du et al., Cell Stem Cell 14: 394-403 (2014)), which is incorporated herein by reference, particularly in parts pertinent to glycogen synthesis capability and/or storage capability assessment.

In exemplary embodiments, the mature-hepatocyte-like cell exhibits lipid (e.g., VLDL, LDL, and HDL) uptake and/or storage capability. In particular embodiments, the mature-hepatocyte-like cell exhibits low-density lipoprotein (LDL) uptake and/or storage capability. In some embodiments, the mature hepatocyte-like cell exhibits at least 10%, 15%, 20%, 25%, 30%, 35%, 40%, 45%, 50%, 55%, 60%, 65%, 70%, 75%, 85%, 90%, 95% or 99% more lipid uptake and/or storage than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of lipid uptake and/or storage of a reference wild-type mature hepatocyte (e.g., a primary human hepatocyte).

In exemplary embodiments, the mature-hepatocyte-like cell exhibits ICG uptake and/or clearance capability. In some embodiments, the mature hepatocyte-like cell at least 10%, 15%, 20%, 25%, 30%, 35%, 40%, 45%, 50%, 55%, 60%, 65%, 70%, 75%, 85%, 90%, 95% or 99% greater ICG uptake and/or clearance than a reference mature hepatocyte-like cell differentiated in the absence of a ureagenesis enhancer. In exemplary embodiments, the mature hepatocyte-like cell exhibits at least 50%, 55%, 60%, 65%, 70%, 75%, 85%, 90%, 95% or 99% the level of lipid uptake and/or storage of a reference wild-type mature hepatocyte (e.g., a primary human hepatocyte). Lipid uptake/storage capability and ICG uptake and/or clearance capability can be assessed using immunohistochemistry techniques as described, for example in Huang et al., Cell Stem Cell 14: 370-384 (2014); and Wang et al., Cell Stem Cell 19: 449-461 (2016), which are incorporated herein by reference, particularly in parts pertinent to ICG activity assessment.

In some embodiments, the mature hepatocyte-like cell exhibits cytochrome p450 activity. Such cells may exhibit activity of one or more cytochrome p450 family members, including CYP1A1, CYP1A2, CYP2C9, CYP2C19, CYP2B6, CYP2D6, CYP3A4, and/or CYP3A7. In certain embodiments, the mature hepatocyte-like cell exhibits gene or protein expression of one or more cytochrome p450 family members. In exemplary embodiments, the mature hepatocyte-like cell exhibits at least 50%, 55%, 60%, 65%, 70%, 75%, 85%, 90%, 95% or 99% the level of cytochrome p450 activity of a reference wild-type mature hepatocyte (e.g., a primary human hepatocyte). Cytochrome p450 activity can be measured using nucleic acid quantitative analysis techniques (e.g., qPCR), luminescent assays (see, e.g., Kim et al., Biomol Ther: 23(5): 486-492 (2015); and P450-Glo assay kit, Promega), or liquid chromatography/mass spectrometry techniques (see, e.g., Du et al., Cell Stem Cell 14: 394-403 (2014); and Lahoz et al., Methods in Molecular Biology 806:97-97 (2012)), all of which are incorporated herein by reference, particularly in parts pertinent to cytochrome p450 activity assessment.

In many embodiments, the mature hepatocyte-like cell exhibits asialoglycoprotein receptor expression (e.g., ASGR1 and/or ASGR2 expression. In exemplary embodiments, the mature hepatocyte-like cell exhibits at least 50%, 55%, 60%, 65%, 70%, 75%, 85%, 90%, 95% or 99% the level of asialoglycoprotein receptor expression of a reference wild-type mature hepatocyte (e.g., a primary human hepatocyte).

In many embodiments, the mature hepatocyte-like cell exhibits alpha-fetoprotein (AFP) expression. In exemplary embodiments, the mature hepatocyte-like cell exhibits at least 50%, 55%, 60%, 65%, 70%, 75%, 85%, 90%, 95% or 99% the level of alpha-fetoprotein expression of a reference wild-type mature hepatocyte (e.g., a primary human hepatocyte).

In some embodiments, the mature hepatocyte-like cell exhibits gamma-glutamyl transpeptidase activity. In exemplary embodiments, the mature hepatocyte-like cell exhibits at least 50%, 55%, 60%, 65%, 70%, 75%, 85%, 90%, 95% or 99% the level of gamma-glutamyl transpeptidase activity of a reference wild-type mature hepatocyte (e.g., a primary human hepatocyte). Gamma-glutamyl transpeptidase activity can be assessed, for example, using immunohistochemistry techniques (see, e.g., Woo et al., Gastroenterology 42:602-611 (2012)), which is incorporated herein by reference, particularly in parts pertinent to gamma-glutamyl transpeptidase activity assessment.

In many embodiments, the mature hepatocyte-like cell exhibits SOX9 expression. In exemplary embodiments, the mature hepatocyte-like cell exhibits at least 50%, 55%, 60%, 65%, 70%, 75%, 85%, 90%, 95%, or 99% the level of SOX9 expression of a reference wild-type mature hepatocyte (e.g., a primary human hepatocyte). In exemplary embodiments, the mature hepatocyte-like cell exhibits increased SOX9 expression compared to a reference wild-type mature hepatocyte.

In many embodiments, the mature hepatocyte-like cell exhibits keratin, type I cytoskeletal 18 (KRT18) expression. In exemplary embodiments, the mature hepatocyte-like cell exhibits at least 50%, 55%, 60%, 65%, 70%, 75%, 85%, 90%, 95%, or 99% the level of KRT18 expression of a reference wild-type mature hepatocyte (e.g., a primary human hepatocyte). In exemplary embodiments, the mature hepatocyte-like cell exhibits increased KRT18 expression compared to a reference wild-type mature hepatocyte.

In many embodiments, the mature hepatocyte-like cell exhibits HNF4A (e.g., HNF4a and HNF4a) expression. In exemplary embodiments, the mature hepatocyte-like cell exhibits at least 50%, 55%, 60%, 65%, 70%, 75%, 85%, 90%, 95%, or 99% the level of HNF4A expression of a reference wild-type mature hepatocyte (e.g., a primary human hepatocyte). In exemplary embodiments, the mature hepatocyte-like cell exhibits increased HNF4A expression compared to a reference wild-type mature hepatocyte.

In many embodiments, the mature hepatocyte-like cell exhibits G6PC expression. In exemplary embodiments, the mature hepatocyte-like cell exhibits at least 50%, 55%, 60%, 65%, 70%, 75%, 85%, 90%, 95% or 99% the level of G6PC expression of a reference wild-type mature hepatocyte (e.g., a primary human hepatocyte). In some embodiments, the mature hepatocyte-like cell exhibits increased G6PC expression compared to a reference wild-type mature hepatocyte.

B. Cell Differentiation

The subject mature hepatocyte-like cells provided herein are produced by in vitro differentiating a source cell in the presence of a ureagenesis enhancer at a discrete time point during the differentiation period.

In exemplary embodiments, the source cell is differentiated into a hepatocyte-like progenitor cell intermediate and the hepatocyte-like progenitor cell is differentiated into a mature-like hepatocyte in a culture medium that includes one or more ureagenesis enhancers. As used herein, a “hepatocyte-like progenitor cell” refers to a cell capable of differentiating into a mature hepatocyte-like cell and that has one or more characteristics of a hepatocyte progenitor cell. Hepatocyte progenitor cell characteristics include, but are not limited to: EpCAM expression, cytokeratin-19 expression, asialoglycoprotein receptor 1 (ASGPR1) expression, and increased gene expression of AFP, ALB, HNF4A, HNF6, SERPINA1, ALB and/or CYP3A7. In particular embodiments, the increased gene expression of AFP, ALB, HNF4A, HNF6, SERPINA1, ALB and/or CYP3A7 is compared to a reference source cell (e.g., a stem cell, a fibroblast or any source cell described herein). In certain embodiments, the increase in gene expression is at least a 10-fold, 100-fold, 1×10³-fold, 1×10⁴-fold, 1×10⁵-fold, or 1×10⁶-fold increase in gene expression as compared to a reference source cell. In exemplary embodiments, the hepatocyte-like progenitor cell exhibits 1) expression of EpCAM and/or cytokeratin-19; and 2) increased gene expression of AFP, ALB, HNF4A, HNF6, SERPINA1, ALB and/or CYP3A7. In some embodiments, the hepatocyte-like progenitor cell exhibits 1) expression of EpCam and cytokeratin-19; and 2) increased expression of ALB and CYP3A7. Hepatocyte-like progenitor cells also include hepatoblasts-like cells, which are bipotential progenitor cells capable of differentiating into hepatocytes or cholangiocytes. In some embodiments, the one or more ureagenesis enhancers is contacted with the mature hepatocyte-like cell.

In some embodiments, one or more ureagenesis enhancers are contacted with the differentiating cell beginning at a specific day of differentiation. In certain embodiments, one or more ureagenesis enhancers are contacted with the differentiating cell beginning at day 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 of differentiation. In some embodiments, one or more ureagenesis enhancers are contacted with the differentiating cell 1, 2, 3, 4, 5, 6, 7, 8, 9, 10, 11, 12, 13, 14, 15, 16, 17, 18, 19, or 20 days prior to differentiation into the mature hepatocyte-like cell. In certain embodiments, one or more ureagenesis enhancers are contacted with a mature hepatocyte-like cell that exhibits one or more characteristics of a mature hepatocyte as disclosed herein. In some embodiments, the ureagenesis enhancer is contacted with the differentiating cell (e.g., the hepatocyte-like progenitor cell) for at least 2, 3, 4, 5, 6, 7, 8, 9, 10, 11, 12, 24 or 36 hours. In some embodiments, the ureagenesis enhancer is contact with the differentiating cell for at least 2, 3, 4, 5, 6, 7, 8, 9, 10, 11, 12, 13, or 14 days. In several embodiments, the ureagenesis enhancer is contact with the differentiating cell for at least 1, 2, 3, 4, 5, 6, 7, 8, 9, or 10 weeks.

1. Source Cells

Any suitable source cell can be used for producing the subject hepatocyte-like cells including, but not limited to, stem cells (e.g., induced pluripotent stem cells and embryonic stem cells) fibroblasts, gastric epithelial cells, hepatocytes, and ductal cells.

In some embodiments, the source cell is a stem cell. In particular embodiments, the source cell is a human stem cell. In some embodiments, the stem cell is a pluripotent stem cell. “Pluripotent stem cells” as used herein have the potential to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art. See, e.g., Zhou et al, Stem Cells 27 (11):2667-74 (2009); Huangfu et al., Nature Biotechnol. 26 (7):795 (2008); Woltjen et al, Nature 458 (7239):766-770 (2009); and Zhou et al, Cell Stem Cell 8:381-384 (2009). In certain embodiments, the stem cell is an embryonic stem cell. Methods of hepatocyte differentiation using stem cells are known in the art. See, e.g., Blackford et al., Stem Cells Transl Med. 8(2):124-137 (2019); Hannan et al., Nature Prot. 8(2):430-437 (2013); Liu et al., Sci Transl Med. 3(82):82ra39 (2011); Woo et al., Gastroenterology 42:602-611 (2012); and Carpentier et al., J Clin Invest. 124(11): 4953-4964 (2014), which are incorporated herein by reference, particularly in parts pertinent to hepatocyte-like cell differentiation methods.

In some of the embodiments, wherein the source cell is an induced pluripotent stem cell, the induced pluripotent stem cell is first differentiated to a definitive endoderm (DE) that expresses one or more of the following genes: CXCR4, SOX17, CER, and/or FOXA2. The DE is then subsequently differentiated into a hepatocyte-like progenitor cell that expresses alpha-1-fetoprotein (AFP). Subsequently, the hepatocyte-like progenitor cell is differentiated into the mature hepatocyte-like cell. See, e.g., Liu et al., Sci Transl Med. 3(82):82ra39 (2011), which is incorporated herein by reference, particularly in parts pertinent to hepatocyte-like cell differentiation. In some embodiments, the hepatocyte-like progenitor cell is differentiated in the presence of one or more of the ureagenesis enhancers described herein to produce the mature hepatocyte-like cell with enhanced ureagenesis capability. In particular embodiments, the hepatocyte-like progenitor cell is differentiated to the mature hepatocyte-like cell in a culture medium that includes one or more ureagenesis enhancers and oncostatin-M (OSM). In certain embodiments, the culture medium further includes hepatocyte growth factor (HGF). In some embodiments, the mature hepatocyte-like cell is subjected to the one or more ureagenesis enhancers.

In some embodiments, wherein the source cell is a human embryonic stem cell or a human induced pluripotent stem cell, the source cell is first differentiated to a definitive endoderm that expresses SOX17 and/or FOXA2. The definitive endoderm is differentiated into a hepatoblast-like cell that expresses AFP and/or HNF4A. The hepatoblast-like cell is then differentiated into a mature hepatocyte-like cell that express one or a combination of the following genes: AFP, AAT, ALB, and HNF3B. See, e.g., Carpentier et al., J Clin Invest. 124(11): 4953-4964 (2014), which is incorporated herein by reference, particularly in parts pertinent to hepatocyte-like cell differentiation. In some embodiments, the hepatoblast-like cell is differentiated in the presence of one or more of the ureagenesis enhancers described herein to produce the mature hepatocyte-like cell with enhanced ureagenesis capability. In certain embodiments, the hepatoblast-like cell is differentiated in a culture medium that includes one or more ureagenesis enhancers, DMSO and HGF. In some embodiments, the mature hepatocyte-like cell is subjected to the one or more ureagenesis enhancers.

In some embodiments, wherein the source cell is a human pluripotent stem cell, the source cell is first differentiated to a definitive endoderm that expresses SOX17 and/or CXCR4. The definite endoderm is differentiated into a hepatic endoderm that expresses EpCAM, cytokeratin 19 and one or a combination of the following genes: AFP, SERPINA2, and HNF4A. The hepatic endoderm is then differentiated into a mature hepatocyte-like cell that express one or a combination of the following genes: AFP, ASGR2, SERPINA2, CYP3A7, and ALB. See, e.g., Blackford et al., Stem Cells Transl Med. 8(2):124-137 (2019), which is incorporated herein by reference, particularly in parts pertinent to hepatocyte-like cell differentiation. In some embodiments, the hepatic endoderm is differentiated in the presence of one or more of the ureagenesis enhancers described herein to produce the mature hepatocyte-like cell with enhanced ureagenesis capability. In certain embodiments, the hepatic endoderm is differentiated in a cell culture medium that includes one or more ureagenesis enhancers and oncostatin-M (OSM). In certain embodiments, the culture medium further includes hepatocyte growth factor (HGF). In some embodiments, the mature hepatocyte-like cell is subjected to the one or more ureagenesis enhancers.

In some embodiments, the mature hepatocyte-like cell is transdifferentiated from a source cell. Transdifferentiation refers to the direct conversion of a differentiated cell type into another without an intermediary pluripotent stage. Exemplary cells that are capable of transdifferentiation into mature hepatocyte-like cells include, but are not limited to, fibroblasts, gastric epithelial cells, hepatocytes, and ductal cells.

In certain embodiments, the mature hepatocyte-like cell is transdifferentiated from a fibroblast. See, e.g., Zhu et al., Nature 508(7494):93-7(2014); Du et al., Cell Stem Cell 14(3):394-403 (2014); and Huang et al., Cell Stem Cell 4(3):370-84, which are incorporated herein by reference, particularly in parts pertinent to methods of transdifferentiation. In such methods, one or more genes useful for fibroblast to hepatocyte reprogramming are transferred to the fibroblast by retroviral transduction delivery techniques and the transduced fibroblast is cultured in medium containing factors favoring the formation of a hepatocyte-like progenitor cell that subsequently differentiates into a mature-hepatocyte like cell. Genes that are useful for fibroblast to hepatocyte reprogramming include, but are not limited to, OCT4, SOX2, KLF4, HNF6, HNF1α, FOXA3, HNF1β and/or HNF4a. In some embodiments, the hepatocyte-like progenitor cell is differentiated in the presence of one or more of the ureagenesis enhancers described herein to produce the mature hepatocyte-like cell with enhanced ureagenesis capability.

In some embodiments, the mature hepatocyte-like cell is transdifferentiated from a mature hepatocyte. In some embodiments, the mature hepatocyte is converted into expandable hepatocyte-like progenitor cells that subsequently differentiate into mature-hepatocyte like cells. In some embodiments, conversion of mature hepatocyte to hepatocyte-like progenitor cell is carried out in a culture medium that includes one or a combination of the following: a Wnt signaling agonist (e.g., CHIR99021), a TGFβ signaling inhibitor (e.g., A82-01 and A83-01) and a ROCK kinase inhibitor (e.g., Y27632, A82-01). See, e.g., Kim et al., J. Hepatol. 70(1):97-107 (2019); and Fu et al., Cell Res. 29(1):8-22 (2019), which are incorporated herein by reference, particularly in parts pertinent to methods of transdifferentiation. In some embodiments, the hepatocyte-like progenitor cell is differentiated in the presence of one or more of the ureagenesis enhancers described herein to produce the mature hepatocyte-like cell with enhanced ureagenesis capability.

In some embodiments, the subject mature hepatocyte-like cell is transdifferentiated from a gastric epithelial cell (e.g., Wang et al., Cell Stem Cell 19(4):449-61 (2016)) or a ductal cell (see, e.g., Huch et al., Cell 160(1-2):299-312 (2015), which are incorporated herein by reference, particularly in parts pertinent to methods of transdifferentiation.). In exemplary embodiments, the source cell is transdifferentiated in the presence of one or more of the ureagenesis enhancers described herein to produce the mature hepatocyte-like cell with enhanced ureagenesis capability.

2. Ureagenesis Enhancers

The subject mature hepatocyte-like cells provided herein are produced by in vitro differentiating a source cell under one or more conditions that promote enhanced in vitro ureagenesis capability.

a. Small Molecule Enhancers

In some embodiments, differentiation occurs in at least one culture medium that includes one or more small molecule ureagenesis enhancers. In exemplary embodiments, such a differentiation step occurs after the source cell has differentiated into an intermediate hepatocyte-like progenitor cell. In certain embodiments, the small molecule enhancer is added to a mature hepatocyte-like cell that exhibits one or more characteristics of a mature hepatocyte as disclosed herein. In some embodiments, differentiation in the presence of the one or more small molecule ureagenesis enhancers occurs 1, 2, 3, 4, 5, 6, 7, 8, 9, 10, 11, 12, 13, 14, 15, 16, 17, 18, 19, or20 days prior to formation of the mature hepatocyte-like cell. In some embodiments, the small molecule ureagenesis enhancer is contact with the differentiating cell for at least 2, 3, 4, 5, 6, 7, 8, 9, 10, 11, 12, 13, or 14 days. In some embodiments, the small molecule ureagenesis enhancer is contact with the differentiating cell for 2, 3, 4, 5, 6, 7, 8, 9, 10, 11, 12, 13, or 14 days. In several embodiments, the small molecule ureagenesis enhancer is contact with the differentiating cell for at least 1, 2, 3, 4, 5, 6, 7, 8, 9, or 10 weeks.

In some embodiments, the small molecule enhancers disclosed herein are included in the culture medium at a concentration of at least 1, 5, 10, 15, 20, 25, 30, 35, 40, 45, 50, 55, 60, 65, 70, 75, 80, 85, 90, 100, 250, 500, or 750 μM. In some embodiments, the small molecule enhancers disclosed herein are included in the culture medium at a concentration of between 1 and 800 μM, between 1 and 750 μM, between 1 and 700 μM, between 1 and 650 μM, between 1 and 600 μM, between 1 and 550 μM, between 1 and 500 μM, between 1 and 500 μM, between 1 and 450 μM, between 1 and 400 μM, between 1 and 350 μM, between 1 and 300 μM, between 1 and 250 μM, between 1 and 200 μM, between 1 and 150 μM, between 1 and 100 μM, between 1 and 50 μM, between 50 and 800 μM, between 50 and 750 μM, between 50 and 700 μM, between 50 and 650 μM, between 50 and 600 μM, between 50 and 550 μM, between 50 and 500 μM, between 50 and 500 μM, between 50 and 450 μM, between 50 and 400 μM, between 50 and 350 μM, between 50 and 300 μM, between 50 and 250 μM, between 50 and 200 μM, between 50 and 150 μM, between 50 and 100 μM, between 100 and 800 μM, between 100 and 750 μM, between 100 and 700 μM, between 100 and 650 μM, between 100 and 600 μM, between 100 and 550 μM, between 100 and 500 μM, between 100 and 500 μM, between 100 and 450 μM, between 100 and 400 μM, between 100 and 350 μM, between 100 and 300 μM, between 100 and 250 μM, between 100 and 200 μM, between 100 and 150 μM, between 200 and 800 μM, between 200 and 750 μM, between 200 and 700 μM, between 200 and 650 μM, between 200 and 600 μM, between 200 and 550 μM, between 200 and 500 μM, between 200 and 500 μM, between 200 and 450 μM, between 200 and 400 μM, between 200 and 350 μM, between 200 and 300 μM, between 200 and 250 μM, between 300 and 800 μM, between 300 and 750 μM, between 300 and 700 μM, between 300 and 650 μM, between 300 and 600 μM, between 300 and 550 μM, between 300 and 500 μM, between 300 and 500 μM, between 300 and 450 μM, between 300 and 400 μM, between 300 and 350 μM, between 400 and 800 μM, between 400 and 750 μM, between 400 and 700 μM, between 400 and 650 μM, between 400 and 600 μM, between 400 and 550 μM, between 400 and 500 μM, between 400 and 500 μM, between 400 and 450 μM, between 500 and 800 μM, between 500 and 750 μM, between 500 and 700 μM, between 500 and 650 μM, between 500 and 600 μM, between 500 and 550 μM, between 600 and 800 μM, between 600 and 750 μM, between 600 and 700 μM, between 600 and 650 μM, between 700 and 800 μM, between 700 and 750 μM, or between 750 and 800 μM.

In some embodiments, the small molecule enhancers disclosed herein are included in the culture medium at a concentration of at least 1, 5, 10, 15, 20, 25, 30, 35, 40, 45, 50, 55, 60, 65, 70, 75, 80, 85, 90, 100, 250, 500, or 750 mM. In some embodiments, the small molecule enhancers disclosed herein are included in the culture medium at a concentration of between 1 and 800 mM, between 1 and 750 mM, between 1 and 700 mM, between 1 and 650 mM, between 1 and 600 mM, between 1 and 550 mM, between 1 and 500 mM, between 1 and 500 mM, between 1 and 450 mM, between 1 and 400 mM, between 1 and 350 mM, between 1 and 300 mM, between 1 and 250 mM, between 1 and 200 mM, between 1 and 150 mM, between 1 and 100 mM, between 1 and 50 mM, between 50 and 800 mM, between 50 and 750 mM, between 50 and 700 mM, between 50 and 650 mM, between 50 and 600 mM, between 50 and 550 mM, between 50 and 500 mM, between 50 and 500 mM, between 50 and 450 mM, between 50 and 400 mM, between 50 and 350 mM, between 50 and 300 mM, between 50 and 250 mM, between 50 and 200 mM, between 50 and 150 mM, between 50 and 100 mM, between 100 and 800 mM, between 100 and 750 mM, between 100 and 700 mM, between 100 and 650 mM, between 100 and 600 mM, between 100 and 550 mM, between 100 and 500 mM, between 100 and 500 mM, between 100 and 450 mM, between 100 and 400 mM, between 100 and 350 mM, between 100 and 300 mM, between 100 and 250 mM, between 100 and 200 mM, between 100 and 150 mM, between 200 and 800 mM, between 200 and 750 mM, between 200 and 700 mM, between 200 and 650 mM, between 200 and 600 mM, between 200 and 550 mM, between 200 and 500 mM, between 200 and 500 mM, between 200 and 450 mM, between 200 and 400 mM, between 200 and 350 mM, between 200 and 300 mM, between 200 and 250 mM, between 300 and 800 mM, between 300 and 750 mM, between 300 and 700 mM, between 300 and 650 mM, between 300 and 600 mM, between 300 and 550 mM, between 300 and 500 mM, between 300 and 500 mM, between 300 and 450 mM, between 300 and 400 mM, between 300 and 350 mM, between 400 and 800 mM, between 400 and 750 mM, between 400 and 700 mM, between 400 and 650 mM, between 400 and 600 mM, between 400 and 550 mM, between 400 and 500 mM, between 400 and 500 mM, between 400 and 450 mM, between 500 and 800 mM, between 500 and 750 mM, between 500 and 700 mM, between 500 and 650 mM, between 500 and 600 mM, between 500 and 550 mM, between 600 and 800 mM, between 600 and 750 mM, between 600 and 700 mM, between 600 and 650 mM, between 700 and 800 mM, between 700 and 750 mM, or between 750 and 800 mM.

In particular embodiments, the small molecule enhancer increases expression of one or more urea cycle pathway enzymes. Exemplary urea cycle pathway enzymes include, but are not limited to, carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin. In particular embodiments, the small molecule enhancer increases expression of one of the following urea cycle enzymes: CPS1, NAGS, ARG1 ASL, ASS1, or OTC. In some embodiments, the small molecule enhancer increases RNA transcription expression of one or more urea cycle pathway enzymes. In exemplary embodiments, the hepatocyte-like cell exhibits increased protein expression of one or more urea cycle pathway enzymes. In some embodiments, the small molecule enhancer increases protein expression of one or more urea cycle pathway enzymes at an amount that is at least 10%, 15%, 20%, 25%, 30%, 35%, 40%, 45%, 50%, 55%, 60%, 65%, 70%, 75%, 85%, 90%, 95% or 99% more than a reference mature hepatocyte-like cell differentiated in the absence of the enhancer.

In some embodiments, the small molecule enhancer is an agent that increases intracellular cyclic AMP. Without being bound by any particular theory of operation and as disclosed in the examples provided herein, agents that increase intracellular cyclic AMP increase transcription and translation of enzymes of the urea cycle, thereby increasing the in vitro ureagenesis capacity of the mature hepatocyte-like cell. Agents that increase intracellular cyclic AMP include, but are not limited to, forskolin, glucagon, glucagon-like peptide-1 (GLP-1), glucose-dependent insulinotropic peptide (GIP), phosphodiesterase inhibitors, and analogs of any of the foregoing. Exemplary phosphodiesterase inhibitors include IBMX (3-isobutyl-1-methylxanthine), theophylline, V11294A, rolipram, milrinone, CDP-840, papaverine, sildenafil, tadalafil, roflumilast, amrinone, cilostazol, and dipyridamole.

In exemplary embodiments, the enhancer is forskolin. In some embodiments, the differentiating hepatocyte-like progenitor cell is differentiated in a cell culture medium that includes forskolin. In exemplary embodiments, a hepatocyte-like cell that exhibits one or more characteristics of a mature hepatocyte as described herein is differentiated in a cell culture medium that includes forskolin. In some embodiments, forskolin is included in the culture medium at a concentration of at least 1, 5, 10, 15, 20, 25, 30, 35, 40, 45, 50, 75, or 100 μM. In certain embodiments, forskolin is included in the culture medium at a concentration of at least 1, 5, 10, 15, 20, 25, 30, 35, 40, 45, 50, 75, or 100 mM. In some embodiments, forskolin is included in the culture medium at a concentration of between 0.5 and 800 μM, between 0.5 and 750 μM, between 0.5 and 700 μM, between 0.5 and 650 μM, between 0.5 and 600 μM, between 0.5 and 550 μM, between 0.5 and 500 μM, between 0.5 and 500 μM, between 0.5 and 450 μM, between 0.5 and 400 μM, between 0.5 and 350 μM, between 0.5 and 300 μM, between 0.5 and 250 μM, between 0.5 and 200 μM, between 0.5 and 150 μM, between 0.5 and 100 μM, between 0.5 and 95 μM, between 0.5 and 90 μM, between 0.5 and 85 μM, between 0.5 and 80 μM, between 0.5 and 75 μM, between 0.5 and 70 μM, between 0.5 and 65 μM, between 0.5 and 60 μM, between 0.5 and 55 μM, between 0.5 and 50 μM, between 0.5 and 45 μM, between 0.5 and 40 μM, between 0.5 and 35 μM, between 0.5 and 30 μM, between 0.5 and 25 μM, between 0.5 and 20 μM, between 0.5 and 15 μM, between 0.5 and 10 μM, between 0.5 and 9 μM, between 0.5 and 8 μM, between 0.5 and 7 μM, between 0.5 and 6 μM, between 0.5 and 5 μM, between 0.5 and 2 μM, between 0.5 and 1 μM, between 1 and 30 μM, between 5 and 30 μM, between 10 and 30 μM, between 1 and 20 μM, between 5 and 20 μM, or between 10 and 20 μM. In some embodiments, forskolin is included in the culture medium at a concentration of 5-20 μM. In some embodiments, the cell (e.g., a differentiating hepatocyte-like progenitor cell) is subjected to forskolin for at least 1, 2, 3, 4, 5, 6, 7, 8, 9, 10, 11, 12, 13, 14, 15, 16, 17, 18, 19, 20, 21, 22, 23, or 24 hours. In exemplary embodiments, the differentiating cell is subjected to forskolin for at least 8 hours. In several embodiments, the cell culture medium further includes Oncostatin-M (OSM) and/or hepatocyte growth factor (HGF).

In some embodiments, the small molecule enhancer is vitamin K1. In exemplary embodiments, the vitamin K is vitamin K1. In some embodiments, differentiation occurs in a culture medium that is substantially free of vitamin K2 and/or vitamin K3. Without being bound by any particular theory of operation and as disclosed in the examples provided herein, vitamin K can increase in vitro ureagenesis in mature hepatocyte-like cells. In some embodiments, a differentiating hepatocyte-like progenitor cell is differentiated in a cell culture medium that includes vitamin K1. In exemplary embodiments, a hepatocyte-like cell that exhibits one or more characteristics of a mature hepatocyte as described herein is differentiated in a cell culture medium that includes vitamin K1. In some embodiments, vitamin K1 is included in the culture medium at a concentration of at least 0.05, 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 100, 250, 500, or 750 μM. In some embodiments, vitamin K is included in the culture medium at a concentration of between 0.05 and 800 μM, between 0.05 and 750 μM, between 0.05 and 700 μM, between 0.05 and 650 μM, between 0.05 and 600 μM, between 0.05 and 550 μM, between 0.05 and 500 μM, between 0.05 and 500 μM, between 0.05 and 450 μM, between 0.05 and 400 μM, between 0.05 and 350 μM, between 0.05 and 300 μM, between 0.05 and 250 μM, between 0.05 and 200 μM, between 0.05 and 150 μM, between 0.05 and 100 μM, between 0.05 and 95 μM, between 0.05 and 90 μM, between 0.05 and 85 μM, between 0.05 and 80 μM, between 0.05 and 75 μM, between 0.05 and 70 μM, between 0.05 and 65 μM, between 0.05 and 60 μM, between 0.05 and 55 μM, between 0.05 and 50 μM, between 0.05 and 45 μM, between 0.05 and 40 μM, between 0.05 and 35 μM, between 0.05 and 30 μM, between 0.05 and 25 μM, between 0.05 and 20 μM, between 0.05 and 15 μM, between 0.05 and 10 μM, between 0.05 and 9 μM, between 0.05 and 8 μM, between 0.05 and 7 μM, between 0.05 and 6 μM, between 0.05 and 5 μM, between 0.05 and 2 μM, between 0.05 and 1 μM, between 0.05 and 0.5 μM, between 0.05 and 0.1 μM, between 0.1 and 5 μM, between 0.5 and 5 μM, or between 1 and 5 μM. In some embodiments, the cell (e.g., a differentiating hepatocyte-like progenitor cell) is subjected to vitamin K1 for at least 1, 2, 3, 4, 5, 6, 7, 8, 9, 10, 11, 12, 13, 14, 15, 16, 17, 18, 19, 20, 21, 22, 23, or 24 hours. In exemplary embodiments, the differentiating cell is subjected to vitamin for at least 8 hours. In some embodiments, arginine is added to the culture medium together with the vitamin K1. In certain embodiments, arginine is added at a concentration of 750 μM-10 mM. In exemplary embodiments, arginine is added to the culture medium at a similar concentration to vitamin K1. In several embodiments, the cell culture medium further includes Oncostatin-M (OSM) and/or hepatocyte growth factor (HGF).

In some embodiments, the small molecule enhancer is vitamin K2. In exemplary embodiments, the vitamin K is vitamin K2. In some embodiments, differentiation occurs in a culture medium that is substantially free of vitamin K1 and/or vitamin K3.

In some embodiments, a differentiating hepatocyte-like progenitor cell is differentiated in a cell culture medium that includes vitamin K2. In exemplary embodiments, a hepatocyte-like cell that exhibits one or more characteristics of a mature hepatocyte as described herein is differentiated in a cell culture medium that includes vitamin K2. In some embodiments, vitamin K2 is included in the culture medium at a concentration of at least 0.05, 0.1, 0.2, 0.3, 0.4, 0.5, 0.75, 1, 2, 3, 4, 5, 10, 15, 20, 25, 30, 35, 40, 45, 50, 55, 60, 65, 70, 75, 80, 85, 90, 100, 250, 500, or 750 μM. In some embodiments, vitamin K is included in the culture medium at a concentration of between 0.05 and 800 μM, between 0.05 and 750 μM, between 0.05 and 700 μM, between 0.05 and 650 μM, between 0.05 and 600 μM, between 0.05 and 550 μM, between 0.05 and 500 μM, between 0.05 and 500 μM, between 0.05 and 450 μM, between 0.05 and 400 μM, between 0.05 and 350 μM, between 0.05 and 300 μM, between 0.05 and 250 μM, between 0.05 and 200 μM, between 0.05 and 150 μM, between 0.05 and 100 μM, between 0.05 and 95 μM, between 0.05 and 90 μM, between 0.05 and 85 μM, between 0.05 and 80 μM, between 0.05 and 75 μM, between 0.05 and 70 μM, between 0.05 and 65 μM, between 0.05 and 60 μM, between 0.05 and 55 μM, between 0.05 and 50 μM, between 0.05 and 45 μM, between 0.05 and 40 μM, between 0.05 and 35 μM, between 0.05 and 30 μM, between 0.05 and 25 μM, between 0.05 and 20 μM, between 0.05 and 15 μM, between 0.05 and 10 μM, between 0.05 and 9 μM, between 0.05 and 8 μM, between 0.05 and 7 μM, between 0.05 and 6 μM, between 0.05 and 5 μM, between 0.05 and 2 μM, between 0.05 and 1 μM, between 0.05 and 0.5 μM, between 0.05 and 0.1 μM, between 0.1 and 5 μM, between 0.5 and 5 μM, or between 1 and 5 μM. In some embodiments, the cell (e.g., a differentiating hepatocyte-like progenitor cell) is subjected to vitamin K2 for at least 1, 2, 3, 4, 5, 6, 7, 8, 9, 10, 11, 12, 13, 14, 15, 16, 17, 18, 19, 20, 21, 22, 23, or 24 hours. In exemplary embodiments, the differentiating cell is subjected to vitamin for at least 8 hours. In some embodiments, arginine is added to the culture medium together with the vitamin K2. In certain embodiments, arginine is added at a concentration of 750 μM-10 mM. In exemplary embodiments, arginine is added to the culture medium at a similar concentration to vitamin K2. In several embodiments, the cell culture medium further includes Oncostatin-M (OSM) and/or hepatocyte growth factor (HGF).

b. Culture Medium

Differentiation to the mature hepatocyte-like cells can occur in the presence of a culture medium that includes one or more growth factors. In some embodiments, the differentiation occurs in a culture medium that includes hepatocyte growth factor (HGF), and Oncostatin-M (OSM). In some embodiments, wherein differentiation occurs through an intermediate hepatocyte-like progenitor cell, the differentiation to the mature hepatocyte-like cell is done in the absence of HGF. In some embodiments, the culture medium includes OSM, but is HGF-free. In particular embodiments, wherein differentiation occurs through an intermediate hepatocyte-like progenitor cell, the differentiation from intermediate hepatocyte-like progenitor cell to the mature hepatocyte-like cell is initially performed in the presence of HGF and the HGF is removed from the culture medium 1, 2, 3, 4, 5, 6, 7, 8, 9, 10, 11, 12, 13, 14, 15, 16, 17, 18, 19, 20, 21, 21, 22, 23, or 24 hours after the start of differentiation. In certain embodiments, the HGF is removed from the culture medium 2, 3, 4, 5, 6, 7, 8, 9, or 10 days from the beginning of differentiation of the hepatocyte-like progenitor cell to the mature hepatocyte-like cell.

c. Increased Oxygen Conditions

Without being bound by anything particular theory of operation and as shown in the examples provided herein, hepatocyte-like cells cultured under elevated oxygen conditions exhibit increased in vitro ureagenesis. Thus, in some embodiments, differentiation to the mature hepatocyte-like cell according to the methods provided herein occurs at an elevated oxygen level that promotes enhanced in vitro ureagenesis. In some embodiments, the differentiation occurs under normoxia (normoxic) conditions (˜20% partial pressure of 02). In certain embodiments, the differentiation occurs under hyperoxia conditions (>20% partial pressure). In some embodiments, the differentiation to mature hepatocyte-like cell is performed at above 5, 6, 7, 8, 9, 10, 11, 12, 13, 14, 15, 16, 17, 18, 19, 20, 21, 22, 23, 24, 25, 26, 27, 28, 29, 30, 35, or 40% partial pressure of 02. In some embodiments, the differentiation to mature hepatocyte-like cell is performed at 5-30%, 6-30%, 7-30%, 8-30%, 9-30%, 10-30%, 11-30%, 12-30%, 13-30%, 14-30%, 15-30%, 16-30%, 17-30%, 18-30%, 19-30%, 20-30%, 21-30%, 22-30%, 23-30%, 24-30%, 25-30%, 26-30%, 27-30%, 28-30%, 29-30%, 15-25%, 15-35%, 15-40%, or 20-25% partial pressure of O₂.

In some embodiments, differentiation to the mature hepatocyte-like cell according to the methods provided herein occurs under at least two oxygen conditions. In some instances, one of the oxygen conditions is a hypoxic condition and the other oxygen condition is a normoxic condition. In some embodiments, the hypoxic condition comprises an oxygen content of about 0% to about 5% oxygen, e.g., about 0%-5%, about 0%-4%, about 0%-3%, about 0%-2%, about 0%-3%, about 0%-4%, about 1%-5%, about 1%-4%, about 1%-3%, about 1%-2%, about 2%-3%, about 3%-4%, about 4%-5%, about 2%-5%, and about 3%-5% oxygen content. In some embodiments, the normoxic condition comprises an oxygen content of about 20% to about 22% oxygen, e.g., about 20%-22%, about 20%-2 1%, about 21%-22%, about 20%, about 21%, and about 22% oxygen content. In some embodiments, the differentiating cells are exposed to a hypoxic condition for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more days. In some embodiments, the differentiating cells are exposed to a normoxic condition for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or more days.

3. Culturing Systems

The differentiation methods described herein can be carried out with cells in suspension or attached to a 2D or 3D solid support. Solid supports include, but are not limited to, glass or plastic culture dishes, multiwell dishes, flasks, plates, microcarriers, polyvinylidene fluoride, polymer films such as metal films and 3D scaffolds. In certain embodiments, the differentiation occurs in a 2D culture system. In some embodiments, the differentiation occurs in a 3D scaffold composed of a synthetic or natural hydrogel. In exemplary embodiments, the 3D scaffold is a poly(ethylene glycol) (PEG) scaffold. In particular embodiments, the 3D scaffold is an inverted colloidal crystal PEG scaffold. See, e.g., Shirahama et al., J Vis Exp. 114:54331 (2016), which is incorporated herein by reference, particularly in parts pertinent to inverted colloidal crystal PEG scaffolds.

In some embodiments, differentiation to mature hepatocyte-like cells is carried out on a 2D or 3D solid support that includes one or more extracellular matrix components. Extracellular matrix components include, but are not limited to, type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin. In exemplary embodiments, the solid support is coated with recombinant laminin (e.g., Laminin 521, BioLamina). In some embodiments, the solid support is free of fetal bovine serum (FBS). In some embodiments wherein differentiation occurs via a hepatocyte-like progenitor cell intermediate, the hepatocyte-like progenitor cell intermediate is transferred to a solid substrate coated with an ECM component that promotes differentiation to the mature hepatocyte-like cell. In some embodiments, the differentiation occurs initially in a fetal bovine serum free first substrate and then the differentiating cells are transferred to a second substrate that includes laminin and/or collagen to promote differentiation to the mature hepatocyte-like cell. In some embodiments, the differentiating cells are transferred to the second substrate at about day 10, 11, 12, 13, 14, 15, 16, 17, 18, 19, 20, 21, 22, 23, or 24 of differentiation. In exemplary embodiments, the first substrate includes gelatin. In some embodiments, the second substrate further includes fetal bovine serum. Exemplary ECM component that promotes differentiation to the mature hepatocyte-like cell include, for example, recombinant Laminin 411 (BioLamina).

In some embodiments, the differentiation is carried out using a hydrogel substrate. Hydrogel substrates can be used with the differentiation methods provided as part of a 2D or 3D culturing system. In particular embodiments, the hydrogel matrix has an elastic modulus of less than 2, 1, 0.5, or 0.1 kPa. In some embodiments, the hydrogel matrix has an elastic modulus of between 10 Pa and 5 kPa, between 10 Pa and 1 kPa, between 8 Pa and 1 kPa, between 50 Pa and 5 kPa, between 100 Pa and 5 kPa, between 200 Pa and 5 kPa, between 250 Pa and 5 kPa, between 500 Pa and 5 kPa, between 750 Pa and 5 kPa, 1 kPa and 5 kPa, between 10 Pa and 5 kPa, between 10 Pa and 1 kPa, between 10 Pa and 900 Pa, between 10 Pa and 800 Pa, between 10 Pa and 700 Pa, between 10 Pa and 600 Pa, between 10 Pa and 500 Pa, between 10 Pa and 400 Pa, between 10 Pa and 300 Pa, between 10 Pa and 200 Pa, between 10 Pa and 100 Pa, between 10 Pa and 50 Pa, or between 10 Pa and 25 Pa. In some embodiments, the hydrogel matrix has an elastic modulus of at least 1, 2, 3, 4, 5, 10, 15, 20, 25, 30, 35, 40, 45, 50 kPa. In some embodiments, the hydrogel matrix has an elastic modulus of between 1 and 100 kPa, between 5 and 100 kPa, between 10 and 100 kPa, between 25 and 100 kPa, between 50 and 100 kPa, between 1 and 100 kPa, between 1 and 50 kPa, between 1 and 40 kPa, between 1 and 30 kPa, between 1 and 25 kPa, between 1 and 20 kPa, between 1 and 15 kPa, between 1 and 10 kPa, between 10 and 50 kPa, between 10 and 40 kPa, between 10 and 35 kPa, between 10 and 30 kPa, between 10 and 25 kPa, between 10 and 20 kPa, between 10 and 15 kPa, between 1 and 10 kPa, between 20 and 50 kPa, between 20 and 40 kPa, between 20 and 35 kPa, between 20 and 30 kPa, between 20 and 25 kPa, between 30 and 50 kPa, between 30 and 40 kPa, between 30 and 35 kPa, or between 40 and 50 kPa. In certain embodiments, the hydrogel matrix has an elastic modulus that prevents Yes Associated Protein 1 (YAP1) from interfering with differentiation to the mature hepatocyte-like cell. It is believed that stiff matrix causes cell spreading in hepatocyte-like progenitor cell via nuclear YAP1. Such cell spreading causes the hepatocyte-like progenitor cell to differentiate into a cholangiocyte and hinders hepatocyte differentiation. Thus, in some embodiments, elastic hydrogel substrates (less than 2 kPa) advantageously promote differentiation to mature hepatocyte-like cells. In some embodiments, the hydrogel substrate is attached to one or more ECM component. In particular embodiments, the ECM component is attached to the hydrogel matrix at a density of at least 50, 75, 100, 150, 200, 250, 300, 400, 450, 500, 600, 700, 800, 900 or 1,000 μM of ECM. In particular embodiments, the ECM component is attached to the hydrogel matrix at a density of less than 50, 75, 100, 150, 200, 250, 300, 400, 450, 500, 600, 700, 800, 900 or 1,000 μM of ECM. In particular embodiments, the ECM component is attached to the hydrogel matrix at a density of at least between 50 and 5,000 μM, between 50 and 4,000 μM between 50 and 3,000 μM, between 50 and 2,000 μM, between 50 and 1,000 μM, between 50 and 500 μM, between 50 and 400 μM, between 1 and 300 μM, between 50 and 200 μM, between 50 and 100 μM, between 50 and 75 μM, between 50 and 50 μM, between 50 and 25 μM, between 50 and 10 μM, between 100 and 5,000 μM, between 100 and 4,000 μM between 100 and 3,000 μM, between 100 and 2,000 μM, between 100 and 1,000 μM, between 100 and 1000 μM, between 100 and 400 μM, between 1 and 300 μM, between 100 and 200 μM, between 250 and 5,000 μM, between 500 and 5,000 μM, between 750 and 5,000 μM, between 1,000 and 5,000 μM, between 2,000 and 5,000 μM, between 3,000 and 5,000 μM, between 4,000 and 5,000 μM, between 250 and 5,000 μM, between 500 and 5,000 μM, between 750 and 5,000 μM, between 1,000 and 5,000 μM, between 2,000 and 5,000 μM, between 3,000 and 5,000 μM, between 4,000 and 5,000 μM, between 100 and 1,000 μM, between 200 and 900 μM, between 300 and 800 μM, between 400 and 700 μM, or between 500 and 600 μM of ECM. In some embodiments, differentiation to mature hepatocyte-like cell occurs in the presence of an elastic hydrogel with a high density of ECM component. Stiff hydrogels that include a low density of ECM components and elastic hydrogels that include a low density of ECM components promote maturation to mature hepatocyte-like cells. In some embodiments, differentiation to mature hepatocyte-like cell occurs in the presence of an elastic hydrogel that includes a high density of ECM component. In some embodiments, differentiation to mature hepatocyte-like cell occurs in the presence of a stiff hydrogel that includes a low density of ECM component.

In some embodiments, the differentiating cell is co-cultured in the presence of Human Umbilical Vein Endothelial Cells (HUVECs). It has been shown that differentiation to mature hepatocyte-like cell enhances in vitro ureagenesis capability. In some embodiments wherein differentiation occurs by a hepatocyte-like progenitor cell intermediate, the hepatocyte-like progenitor cell is cultured in the presence of the HUVECs. In some embodiments, the hepatocyte-like progenitor cell is in direct contact with the HUVECs in the culture. In other embodiments, the hepatocyte-like progenitor cell and HUVECs are cultured in two different compartments separated by a porous membrane (e.g., a transwell culture system).

4. Hepatocyte Differentiation Protocol in 2D Adherent Cultures

Provided herein is a method for generating pluripotent stem cell-derived hepatocyte-like cells using a 2D adherent culture system. The method includes single-cell passaging and xeno-free components. In some embodiments, pluripotent stem cells are maintained in a cell culture media to maintain pluripotency prior to differentiation.

In some embodiments, the pluripotent stem cells are cultured in a media comprising one or more factors including, but not limited to, activin A, bFGF, BMP4, LY294002, CHIR99021 on day 0. In many embodiments, the pluripotent stem cells are cultured in a first media comprising activin A, bFGF, BMP4, LY294002, CHIR99021 on day 0. In some instances, the pluripotent stem cells are cultured on one or more components of the ECM, including those described herein.

In some embodiments, the differentiating cells are cultured in a media comprising one or more factors including, but not limited to, activin A, bFGF, BMP4, and LY294002 on day 1. In many embodiments, the differentiating cells are cultured in a second media comprising activin A, bFGF, BMP4, and LY294002 on day 1. In many embodiments, the cultured cells at day 1 form networks of cells.

In some embodiments, the differentiating cells are cultured in a media comprising one or more factors including, but not limited to, activin A and bFGF on day 2. In many embodiments, the differentiating cells are cultured in a third media comprising activin A and bFGF on day 2. In some instances, the media further includes a B27 supplement. In many embodiments, the cultured cells at day 2 exhibit a definitive endoderm-like cell morphology and/or activity.

In some embodiments, the differentiating cells are cultured in a media comprising one or more factors including, but not limited to, activin A on days 3-7. In many embodiments, the differentiating cells are cultured in a fourth media comprising activin A on days 3-7. In some embodiments, the cultured cells at days 2-4 exhibit a mesendoderm-like cell morphology and/or activity. In other embodiments, the cultured cells at days 5-7 exhibit a definitive endoderm-like cell morphology and/or activity. In some embodiments, the cultured cells at about days 6-7 comprise hepatoblast-like cells. In certain embodiments, the cultured cells at days 6-7 exhibit a hepatoblast-like cell morphology and/or activity.

In some embodiments, the differentiating cells are cultured in a media comprising one or more factors including, but not limited to, oncostatin-M and HGF on days 8-27. In many embodiments, the differentiating cells are cultured in a fifth media comprising oncostatin-M and HGF on days 8-27. In some instances, the media further includes a B27 supplement. In some embodiments, the cultured cells at days 8-10 exhibit a defined cuboidal morphology. In various embodiments, the cultured cells at days 13-15 exhibit a polyhedral morphology. In some embodiments, at days 13-15 of the differentiation method, the cells exhibit a hepatic endoderm cell morphology and/or activity.

In some embodiments, the differentiating cells are cultured in a media comprising one or more factors including, but not limited to, oncostatin-M, HGF, and forskolin on days 28-35. In many embodiments, the differentiating cells are cultured in a sixth media comprising oncostatin-M, HGF, and forskolin on days 28-35. In some instances, the media further includes chemically defined lipids and a mixture of insulin, transferrin, and selenium.

In some embodiments, at days 20-28 the cells exhibit a hepatocyte-like cell (immature hepatocyte-like cell) morphology and/or activity. In some embodiments, at days 25-35 the cells exhibit a hepatocyte-like cell (mature hepatocyte-like cell) morphology and/or activity. In some embodiments, at days 20-35 the cells comprise hepatocyte-like cells including immature hepatocyte-like cells, mature hepatocyte-like cells, or both. In some embodiments, the cells differentiated according to the method of the present technology comprise at least 80%, 81, 82%, 83%, 84%, 85%, 86%, 87%, 88%, 89%, 90%, 91%, 92%, 93%, 94%, 95%, 96%, 97%, 98%, 99% or more immature hepatocyte-like cells. In some embodiments, the cells differentiated according to the method of the present technology comprise at least 80%, 81, 82%, 83%, 84%, 85%, 86%, 87%, 88%, 89%, 90%, 91%, 92%, 93%, 94%, 95%, 96%, 97%, 98%, 99% or more mature hepatocyte-like cells.

In some embodiments, the mature hepatocyte-like cells are cultured in a media for maintaining and/or expanding hepatocytes such as but not limited to primary hepatocytes. Any of the media compositions described herein can also contain components that are essential for maintain healthy cells including, but not limited to, non-essential amino acids, glutamine, and analogs thereof.

In some embodiments, the pluripotent stem cells are cultured in a first media comprising activin A, bFGF, BMP4, LY294002, CHIR99021 on day 0. After which in some embodiments, the cells are cultured in a second media comprising activin A, bFGF, BMP4, and LY294002 on day 1. After which in some embodiments, the differentiating cells are cultured in a third media comprising activin A and bFGF on day 2. After which in some embodiments, the differentiating cells are cultured in a fourth media comprising activin A on days 3-7. After which in some embodiments, the differentiating cells are cultured in a fifth media comprising oncostatin-M and HGF on days 8-27. After which in some embodiments the differentiating cells are cultured in a sixth media comprising oncostatin-M, HGF and forskolin on days 28-35.

In some embodiment, LY294002 is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 M, about 7 μM, about 8 μM, about 9 μM, about 10 μM, about 11 μM, about 12 μM, about 13 M, about 14 μM, about 15 μM, about 16 μM, about 17 μM, about 18 μM, about 19 μM, or about 20 μM. In many embodiments, the media comprises LY294002 at a concentration of 10 M.

In some embodiment, CHIR-99021 is present at a concentration of about 1-15 μM, about 1-10 μM, about 1-8 μM, about 1-5 μM, about 2-15 μM, about 2-10 PM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM. In many embodiments, the media comprises CHIR-99021 at a concentration of 3 μM.

In some embodiment, activin A is present at a concentration of about 20 ng/ml-200 ng/ml, about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 20 ng/ml-100 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In many embodiments, the media comprises activin A at a concentration of 50 ng/ml or 100 ng/ml.

In some embodiment, bFGF is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In many embodiments, the media comprises bFGF at a concentration of 80 ng/ml

In some embodiment, BMP4 is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In many embodiments, the media comprises BMP4 at a concentration of 10 ng/ml.

In some embodiment, OSM is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-50 ng/ml, about 5 ng/ml-40 ng/ml, about 5 ng/ml-30 ng/ml, about 30 ng/ml-40 ng/ml, about 5 ng/ml-20 ng/ml, or about 30 ng/ml-50 ng/ml. In many embodiments, the media comprises OSM at a concentration of 10 ng/ml.

In some embodiment, HGF is present at a concentration of about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In many embodiments, the media comprises HGF at a concentration of 50 ng/ml.

In some embodiment, forskolin is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 M, about 7 μM, about 8 μM, about 9 μM, about 10 μM, about 11 μM, about 12 μM, about 13 M, about 14 μM, about 15 μM, about 16 μM, about 17 μM, about 18 μM, about 19 μM, or about 20 μM. In many embodiments, the media comprises forskolin at a concentration of 10 M.

5. Hepatocyte Differentiation Protocol in 3D Suspension Cultures

Provided herein is a method for generating pluripotent stem cell-derived hepatocyte-like cells using a 3D suspension culture system. In some embodiments, pluripotent stem cells are maintained in a cell culture media to maintain pluripotency prior to differentiation.

In some embodiments, the cells are cultured in a media comprising one or more factors including, but not limited to, insulin, PIK-90, activin A, bFGF, BMP4, and CHIR99021 on day 0. In many embodiments, the pluripotent stem cells are cultured in a first media comprising insulin, PIK-90, activin A, bFGF, BMP4, and CHIR99021 on day 0. In some instances, the pluripotent stem cells are cultured on a solid support that facilitate low or no attachment to the support surface, including those described herein. In some instances, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health.

In some embodiments, the cells are cultured at a cell concentration (cell density) of about 500,000 to about 5,000,000 cells per mL, e.g., about 500,000-5,000,000; about 500,000-4,500,000; about 500,000-4,000,000; about 500,000-3,500,000; about 500,000-3,000,000; about 500,000-2,500,000; about 500,000-2,000,000; about 500,000-2,500,000; about 500,000-1,500,000; about 500,000-1,000,000; about 1,000,000-5,000,000; about 1,000,000-4,500,000; about 1,000,000-4,000,000; about 1,000,000-3,500,000; about 1,000,000-3,000,000; about 1,000,000-2,500,000; about 1,000,000-2,000,000; about 1,000,000-1,500,000 cells per mL.

In some embodiments, the cells are cultured under a hypoxic condition. In some embodiments, the hypoxic condition comprises an oxygen content of about 0% to about 5% oxygen, e.g., about 0%-5%, about 0%-4%, about 0%-3%, about 0%-2%, about 0%-3%, about 0%-4%, about 1%-5%, about 1%-4%, about 1%-3%, about 1%-2%, about 2%-3%, about 3%-4%, about 4%-5%, about 2%-5%, and about 3%-5% oxygen content. In some embodiments, the cells from into aggregates. Exemplary examples of such aggregates are provided in the Figures.

In some embodiments, the cell aggregates are between about 100-250 m in diameter, e.g., about 100-250, about 100-240, about 100-230, about 100-220, about 100-210, about 100-200, about 110-250, about 120-250, about 130-250, about 140-250, about 150-250, about 160-250, about 100-200, about 100-190, about 100-180, about 100-170, about 140-250, about 140-250, about 140-240, about 140-230, about 140-220, about 140-210, about 140-200, about 140-190 m in diameter.

In some embodiments, the differentiating cell aggregates are cultured in a media comprising one or more factors including, but not limited to, insulin, PIK-90, activin A, bFGF, and BMP4 on day 1. In many embodiments, the cell aggregates are cultured in a second media comprising insulin, PIK-90, activin A, bFGF, and BMP4 on day 1. In some instances, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health. In some embodiments, the cells outlined are cultured under a hypoxic condition from days 0-21.

In some embodiments, the differentiating cell aggregates are cultured in a media comprising one or more factors including, but not limited to, insulin, PIK-90, activin A, and bFGF on day 2. In many embodiments, the cell aggregates are cultured in a third media comprising insulin, PIK-90, activin A, and bFGF on day 2. In some instances, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health.

In some embodiments, the differentiating cell aggregates are cultured in a media comprising one or more factors including, but not limited to, B27 supplement and activin A on days 3-5. In many embodiments, the cell aggregates are cultured in a fourth media comprising B27 supplement and activin A on days 3-5. In some instances, the media further includes some embodiments, the media also includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health. The media can be refreshed, changed, or replaced daily, every other day, or as needed.

In some embodiments, the differentiating cell aggregates are cultured in a media comprising one or more factors including, but not limited to, B27 supplement, BMP4, and FGF10 on days 6-9. In many embodiments, the cell aggregates are cultured in a fifth media comprising B27 supplement, BMP4, and FGF10 on days 6-9. In some instances, the media further includes one or more of the following components: human serum albumin, chemically defined lipids, non-essential amino acids, ascorbic acid-2-phosphate, holo-transferrin, sodium selenite, and any additional reagent for facilitating hepatocyte differentiation and/or cell health. The media can be refreshed, changed, or replaced daily, every other day, or as needed.

In some embodiments, the differentiating cell aggregates are cultured in a media comprising one or more factors including, but not limited to, HGF on days 10-14. In many embodiments, the cell aggregates are cultured in a sixth media comprising HGF on days 10-14. In some instances, the media further includes chemically defined lipids and a mixture of insulin, transferrin, and selenium. The media can be refreshed, changed, or replaced daily, every other day, or as needed.

In some embodiments, the differentiating cells are cultured in a media comprising one or more factors including, but not limited to, oncostatin-M on days 15-27. In many embodiments, the cell aggregates are cultured in a seventh media comprising oncostatin-M on days 15-27. In many embodiments, the media at this stage of differentiation is free of HGF. In some instances, the media further includes chemically defined lipids and a mixture of insulin, transferrin, and selenium. The media can be refreshed, changed, or replaced daily, every other day, or as needed.

In some embodiments, the differentiating cells are cultured in a media comprising one or more factors including, but not limited to, oncostatin-M and forskolin on days 28-35. In many embodiments, the cell aggregates are cultured in a eighth media comprising oncostatin-M and forskolin on days 28-35. In many embodiments, the media at this stage of differentiation is free of HGF. In some instances, the media further includes chemically defined lipids and a mixture of insulin, transferrin, and selenium. The media can be refreshed, changed, or replaced daily, every other day, or as needed.

In some embodiments, at days 15-35 the cells comprise hepatocyte-like cells including immature hepatocyte-like cells, mature hepatocyte-like cells, or both. In some embodiments, the cells differentiated according to the method of the present technology comprise at least 80%, 81, 82%, 83%, 84%, 85%, 86%, 87%, 88%, 89%, 90%, 91%, 92%, 93%, 94%, 95%, 96%, 97%, 98%, 99% or more immature hepatocyte-like cells. In some embodiments, the cells differentiated according to the method of the present technology comprise at least 80%, 81, 82%, 83%, 84%, 85%, 86%, 87%, 88%, 89%, 90%, 91%, 92%, 93%, 94%, 95%, 96%, 97%, 98%, 99% or more mature hepatocyte-like cells.

In some embodiments, the cell aggregates are cultured under a normoxic condition (normoxia). In some embodiments, the normoxic condition comprises an oxygen content of about 20% to about 22% oxygen, e.g., about 20%-22%, about 20%-2 1%, about 21%-22%, about 20%, about 21%, and about 22% oxygen content. In other words, normoxic condition includes ambient levels of O₂. In some instances, the cell aggregates are cultured under a normoxic condition at least days 15-27, 21-27, 20-28, 20-35, 27-35, 28-35, 29-35, or 30-35.

In some embodiments, the pluripotent stem cells are cultured in a first media comprising insulin, PIK-90, activin A, bFGF, BMP4, and CHIR99021 on day 0. On day 0 the cells from aggregates (also referred to as spheroids). After which in some embodiments, the cell aggregates are cultured in a second media comprising insulin, PIK-90, activin A, bFGF, and BMP4 on day 1. After which in some embodiments, the cell aggregates are cultured in a third media comprising insulin, PIK-90, activin A, and bFGF on day 2. After which in some embodiments, the cell aggregates are cultured in a fourth media comprising B27 supplement and activin A on days 3-5. After which in some embodiments, the cell aggregates are cultured in a fifth media comprising B27 supplement, BMP4, and FGF10 on days 6-9. After which in some embodiments, the cell aggregates are cultured in a sixth media comprising HGF on days 10-14. After which in some embodiments, the cell aggregates are cultured in a seventh media comprising oncostatin-M on days 15-27. After which in some embodiments, the cell aggregates are cultured in a eighth media comprising oncostatin-M and forskolin on days 28-35.

In some embodiments, the differentiating cells exhibit definite endoderm cell characteristics at days 0-2. In some embodiments, the differentiating cells exhibit anterior definite endoderm cell characteristics at days 3-5. In some embodiments, the differentiating cells exhibit foregut endoderm cell characteristics at days 6-9. In some embodiments, the differentiating cells exhibit hepatic endoderm cell characteristics at days 10-14. In some embodiments, the differentiating cells exhibit immature hepatocyte-like cell characteristics at days 15-35.

In some embodiment, insulin is present at a concentration of about 0.1 μg/ml, about 0.2 g/ml, about 0.3 μg/ml, about 0.4 μg/ml, about 0.5 μg/ml, about 0.6 μg/ml, about 0.1 μg/ml-about 0.6 μg/ml, about 0.1 μg/ml-about 0.5 μg/ml, about 0.1 μg/ml-about 0.4 μg/ml, about 0.2 g/ml-about 0.6 μg/ml, about 0.2 μg/ml-about 0.5 μg/ml, or about 0.2 μg/ml-about 0.4 μg/ml. In many embodiments, the media comprises insulin at a concentration of 0.2 μg/ml.

In some embodiment, PIK-90 is present at a concentration of about 0.1 μM, about 0.2 M, about 0.3 μM, about 0.4 μM, about 0.5 μM, about 0.6 μM, about 0.1 μM-about 0.6 μM, about 0.1 μM-about 0.5 μM, about 0.1 μM-about 0.4 μM, about 0.2 μM-about 0.6 μM, about 0.2 μM-about 0.5 μM, or about 0.2 μM about 0.4 μM. In many embodiments, the media comprises PIK-90 at a concentration of 0.1 μM.

In some embodiment, CHIR-99021 is present at a concentration of about 1-15 μM, about 1-10 μM, about 1-8 μM, about 1-5 μM, about 2-15 μM, about 2-10 PM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM. In many embodiments, the media comprises CHIR-99021 at a concentration of 3 μM.

In some embodiment, activin A is present at a concentration of about 20 ng/ml-200 ng/ml, about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 20 ng/ml-100 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In many embodiments, the media comprises activin A at a concentration of 50 ng/ml or 100 ng/ml.

In some embodiment, bFGF is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In many embodiments, the media comprises bFGF at a concentration of 10 ng/ml or 40 ng/ml

In some embodiment, BMP4 is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-40 ng/ml, about 5 ng/ml-30 ng/ml, about 10 ng/ml-40 ng/ml, about 5 ng/ml-20 ng/ml, or about 10 ng/ml-50 ng/ml. In many embodiments, the media comprises BMP4 at a concentration of 10 ng/ml or 20 ng/ml.

In some embodiment, OSM is present at a concentration of about 5 ng/ml, about 7 ng/ml, about 8 ng/ml, about 10 ng/ml, about 13 ng/ml, about 15 ng/ml, about 17 ng/ml, about 18 ng/ml, about 20 ng/ml, about 23 ng/ml, about 25 ng/ml, about 27 ng/ml, about 28 ng/ml, about 30 ng/ml, about 35 ng/ml, about 37 ng/ml, about 38 ng/ml, about 40 ng/ml, about 43 ng/ml, about 45 ng/ml, about 47 ng/ml, about 48 ng/ml, about 50 ng/ml, about 51 ng/ml, about 52 ng/ml, about 53 ng/ml, about 55 ng/ml, about 58 ng/ml, about 5 ng/ml-50 ng/ml, about 5 ng/ml-40 ng/ml, about 5 ng/ml-30 ng/ml, about 30 ng/ml-40 ng/ml, about 5 ng/ml-20 ng/ml, or about 30 ng/ml-50 ng/ml. In many embodiments, the media comprises OSM at a concentration of 30 ng/ml.

In some embodiment, HGF is present at a concentration of about 25 ng/ml-200 ng/ml, about 30 ng/ml-200 ng/ml, about 40 ng/ml-200 ng/ml, about 50 ng/ml-200 ng/ml, about 60 ng/ml-200 ng/ml, about 70 ng/ml-200 ng/ml, about 80 ng/ml-200 ng/ml, about 90 ng/ml-200 ng/ml, about 100 ng/ml-200 ng/ml, about 30 ng/ml-100 ng/ml, about 40 ng/ml-100 ng/ml, about 50 ng/ml-100 ng/ml, about 60 ng/ml-100 ng/ml, about 70 ng/ml-100 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml. In many embodiments, the media comprises HGF at a concentration of 50 ng/ml.

In some embodiment, forskolin is present at a concentration of about 1-20 μM, about 1-15 μM, about 1-10 μM, about 1-8 μM, about 1-5 μM, about 2-15 μM, about 2-10 μM, about 2-8 μM, about 1-5 μM, about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In many embodiments, the media comprises forskolin at a concentration of 10 μM.

C. Pharmaceutical Compositions

Any of the hepatocyte-like cells produced by the methods described herein can be used treat a subject. In one aspect, provided herein are pharmaceutical compositions that include the subject mature hepatocyte-like cells with enhanced in vitro ureagenesis capability. In some embodiments, the ureagenesis capability of the mature hepatocyte-like cells is assessed prior to use in a pharmaceutical composition. Ureagenesis can be measured using any suitable assay known in the art, for example, colorimetric urea assays (e.g., QuantiCrom Assay Kit by BiosAssay Systems). In exemplary embodiments, the pharmaceutical composition includes mature hepatocyte-like cell that exhibits at least 50%, 55%, 60%, 65%, 70%, 75%, 85%, 90%, 95% or 99% ureagenesis as compared to a reference wild-type mature hepatocyte.

In certain embodiments, isolated or purified hepatocyte-like cells are present within a composition adapted for and suitable for delivery, i.e., physiologically compatible. In some embodiments, the pharmaceutical composition includes one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In other embodiments, the isolated or purified cell populations are present within a composition adapted for or suitable for freezing or storage.

In certain embodiments, the purity of the cells for administration to a subject is about 100%. In other embodiments, it is 95% to 100%. In some embodiments. it is 85% to 95%. Particularly in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-9 0%, or 90%-95%. Isolation/purity can also be expressed in terms of cell doublings where the cells have undergone, for example, 10-20, 20-30, 30-40, 40-50 or more cell doublings.

The numbers of hepatocyte-like cells in a given volume can be determined by well-known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The choice of formulation for administering the hepatocyte-like cells for a given application will depend on a variety of factors including, but not limited to, species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, preferably succinylated collagen, can also act as lubricants. Such lubricants are generally used to improve the injectability, intrudability and dispersion of the injected biomaterial at the site of injection and to decrease the amount of spiking by modifying the viscosity of the compositions. This final formulation is by definition the cells in a pharmaceutically acceptable carrier.

A pharmaceutically acceptable preservative or stabilizer can be employed to increase the life of cell/medium compositions. If such preservatives are included, it is well within the purview of the skilled artisan to select compositions that will not affect the viability or efficacy of the cells.

Sterile injectable solutions can be prepared by incorporating the cells/medium utilized in practicing the present technology in the required amount of the appropriate solvent with various amounts of the other ingredients, as desired.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

In some embodiments, cells are encapsulated for administration, particularly where encapsulation enhances the effectiveness of the therapy, or provides advantages in handling and/or shelf life. Encapsulation in some embodiments where it increases the efficacy of cell mediated immunosuppression may, as a result, also reduce the need for immunosuppressive drug therapy.

Also, encapsulation in some embodiments provides a barrier to a subject's immune system that may further reduce a subject's immune response to the cells (which generally are not immunogenic or are only weakly immunogenic in allogeneic transplants), thereby reducing any graft rejection or inflammation that might occur upon administration of the cells.

Cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval. In some embodiments, the cell is encapsulated by a polymer matrix. In exemplary embodiments, the polymer matrix is includes alginate, a modified alginate, chitosan, hydroxyethyl methacrylate, methyl methacrylate, agarose, collagen, polylysine, polyethersulfone, polysulfone, polyornithine, and/or aminopropylsilicate or combinations thereof.

A wide variety of materials may be used in various embodiments for microencapsulation of cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. In some embodiments, the hepatocyte-like cells are encapsulated in an alginate capsule. See, e.g., Kerby et al., Artif Organs 36:564-590 (2012); and Kerby et al., Nat Protoc 8:430-437 (2013). All of the foregoing are incorporated herein by reference, particularly in parts pertinent to alginate encapsulation of cells.

Techniques for microencapsulation of cells that may be used for administration of cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference, particularly in parts pertinent to encapsulation of cells.

Certain embodiments incorporate cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines, can also be incorporated into the polymer. In other embodiments, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

In some embodiments, 3D spheroids of the hepatocyte-like cells are generated prior to encapsulation. In certain embodiments, the 3D spheroid includes at least 1×10², 1×10³, 1×10⁴, 1×10⁵, 1×10⁶, 1×10⁷, or 1×10⁸ cells. In certain embodiments, the 3D spheroid includes between 1×10² and 1×10¹⁰ cells, between 1×10³ and 1×10⁹ cells, between 1×10⁴ and 1×10⁸ cells, between 1×10⁵ and 1×10⁷ cells, between 1×10² and 1×10³ cells, between 1×10³ and 1×10⁴ cells, between 1×10⁴ and 1×10⁵ cells, between 1×10⁵ and 1×10⁶ cells, between 1×10⁶ and 1×10⁷ cells, between 1×10⁷ and 1×10⁸ cells, between 1×10⁹ and 1×10¹⁰ cells, between 1×10² and 1×10⁵ cells, between 1×10³ and 1×10⁶ cells, between 1×10⁴ and 1×10⁷ cells, between 1×10⁵ and 1×10⁸ cells, between 1×10⁶ and 1×10⁹ cells, or between 1×10⁷ and 1×10¹⁰ cells. Methods of 3D spheroid formation of hepatocyte-like cells are disclosed, for example, in Blackford et al., Stem Cells Transl Med. 8(2):124-137 (2019), which is incorporated herein by reference, particularly in parts pertinent to methods of 3D spheroid formation.

In the case of treating liver deficiency, in particular, the cells may be enclosed in a device that can be implanted in a subject. Cells can be implanted in or near the liver or elsewhere to replace or supplement liver function. Cells can also be implanted without being in a device, e.g., in existing liver tissue.

D. Methods of Treatment

In another aspect, provided herein are methods of treatment using the subject mature hepatocyte-like cells. In some embodiments, the hepatocyte-like cells are used to restore a degree of liver function to a subject needing such therapy, perhaps due to an acute, chronic, or inherited impairment of liver function. In exemplary embodiments, the mature hepatocyte-like cell used in the method of treatment exhibits at least 50%, 55%, 60%, 65%, 70%, 75%, 85%, 90%, 95% or 99% ureagenesis as compared to a reference wild-type mature hepatocyte.

To determine the suitability of hepatocytes provided herein for therapeutic applications, the cells can first be tested in a suitable animal model. At one level, cells are assessed for their ability to survive and maintain their phenotype in vivo. Hepatocytes provided herein are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether starting cell types such as pluripotent stem cells are still present. Where hepatocyte-like cells provided herein are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. General descriptions for determining the fate of hepatocyte-like cells in animal models is provided in Grompe et al. (1999); Peeters et al., (1997); and Ohashi et al. (2000).

The hepatocyte-like cells provided herein are assessed for their ability to restore liver function in an animal lacking full liver function. Acute liver disease can be modeled by 90% hepatectomy (Kobayashi et al., 2000). Acute liver disease can also be modeled by treating animals with a hepatotoxin such as galactosamine, CCl₄, or thioacetamide.

Chronic liver diseases such as cirrhosis can be modeled by treating animals with a sub-lethal dose of a hepatotoxin long enough to induce fibrosis (Rudolph et al., 2000). Assessing the ability of hepatocytes provided herein to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

Hepatocyte-like cells that demonstrate desirable functional characteristics according to their profile of metabolic enzymes, or efficacy in animal models, may also be suitable for direct administration to human subjects with impaired liver function. For purposes of hemostasis, the cells can be administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In one embodiment, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another embodiment, the cells are administered by placing a bolus in a cavity near the target organ, typically in an excipient or matrix that will keep the bolus in place. In another embodiment, the cells are injected directly into a lobe of the liver or the spleen.

The hepatocyte-like cells provided herein can be used for therapy of any subject in need of having hepatic function restored or supplemented. Human conditions that may be appropriate for such therapy include, but are not limited to, fulminant hepatic failure due to any cause, viral hepatitis, drug-induced liver injury, cirrhosis, inherited hepatic insufficiency (such as Wilson's disease, Gilbert's syndrome, or ai-antitrypsin deficiency), hepatobiliary carcinoma, autoimmune liver disease (such as autoimmune chronic hepatitis or primary biliary cirrhosis), and any other condition that results in impaired hepatic function.

In another aspect, the hepatocyte-like cells provided herein are encapsulated or part of a bioartificial liver device. Bioartificial organs for clinical use are designed to support an individual with impaired liver function-either as a part of long-term therapy, or to bridge the time between a fulminant hepatic failure and hepatic reconstitution or liver transplant. Bioartificial liver devices are disclosed, for example, in U.S. Pat. Nos. 5,290,684, 5,624,840, 5,837,234, 5,853,717, and 5,935,849. Suspension-type bioartificial livers comprise cells suspended in plate dialysers, microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has an inlet and outlet through which the subject's blood is passed, and sometimes a separate set of ports for supplying nutrients to the cells.

Hepatocytes are prepared according to the methods described herein, and then plated into the device on a suitable substrate, such as a matrix of Matrigel® or collagen. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the hepatocytes provided in certain aspects of this technology under conditions that permit the cell to remove or modify a toxin in the fluid. The detoxification will involve removing or altering at least one ligand, metabolite, or other compound (either natural and/or synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. The device can also be used to enrich the efferent fluid with synthesized proteins such as albumin, acute phase reactants, and unloaded carrier proteins. The device can be optimized so that a variety of these functions is performed, thereby restoring as many hepatic functions as are needed. In the context of therapeutic care, the device processes blood flowing from a patient in hepatocyte failure, and then the blood is returned to the patient.

### EXAMPLES

### Example 1. Differentiation of Hepatocyte-Like Cells from Human iPSCs Using Forskolin

Human iPSCs were differentiated to hepatocyte-like cells essentially as described in Hannan et al., Nature Prot. 8(2):430-437 (2013) (Protocol A) or Blackford et al., Stem Cells Transl Med. 8(2):124-137 (2019) (Protocol B) with the following modifications: cells were maintained in the final medium until a total culture time of 35 days and HepatoZYME basal medium was substituted in Protocol B starting at day 8. Forskolin was added to the final medium at a concentration of 10 mM on day 21 (Protocol A) or day 28 (Protocols A and B). Expression of hepatocyte and urea cycle genes was measured by qPCR and/or fluorescent Western blot analysis. Ureagenesis of the cells was assayed using Urea Assay Kit (BioChain).

Addition of forskolin to the medium at days 21-28 of Protocol A had no significant effect on expression of hepatocyte-like cell genes (HNF4a, AFP, ALB, ASGR1) as compared to cells without forskolin treatment (qPCR). By contrast, a significant increase (5-8-fold) in the urea cycle gene CPS1 was observed, with more modest increases in other urea cycle genes NAGS, ARG1, and ASS1.

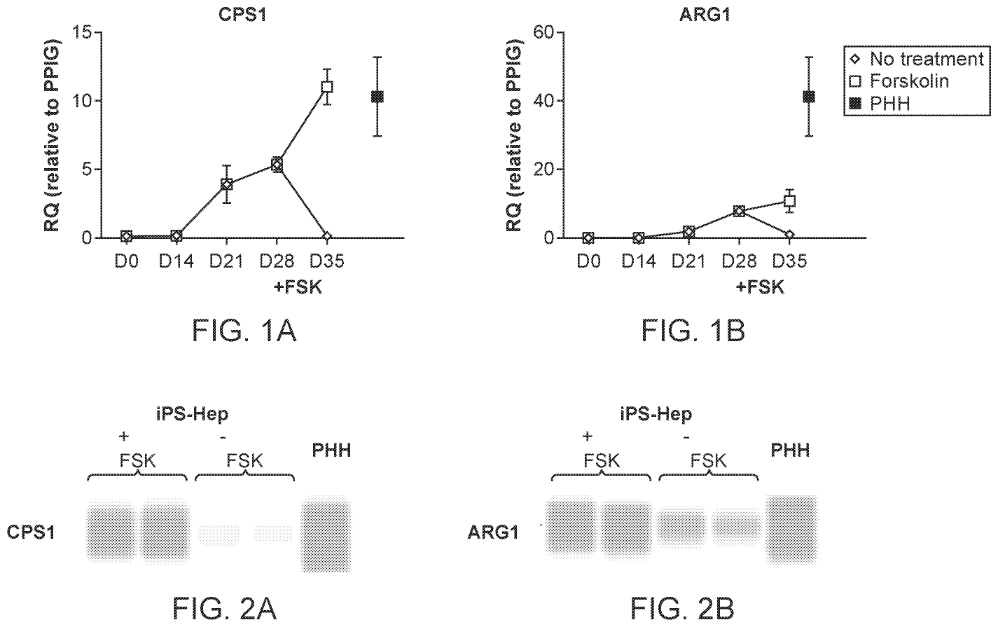

Addition of forskolin to the medium at days 28-35 of Protocol B showed a significant enhancement (2-4-fold) of hepatocyte-like cell gene (HNF4a, ALB, and ASGR1) expression at day 35 as compared to cells without forskolin treatment. Expression of urea cycle genes CPS1 and ARG1 was significantly increased at day 35 (FIGS. 1A and 1B, respectively), and protein expression of CPS1 and ARG1 was similarly increased (FIGS. 2A and 2B, respectively). Addition of forskolin to the medium at days 28-35 of Protocol A showed similar increases in CPS1 and ARG1 expression by qPCR.

Ureagenesis activity was measured in day 35 cells from Protocol A and Protocol B following seven-day treatment with forskolin, following challenge of the hepatocyte-like cells with 0-16 mM ammonia. Forskolin-treated cells from both protocols exhibited ammonia-stimulated ureagenesis, whereas cells without forskolin treatment had no stimulation of ureagenesis. For Protocol A, ureagenesis activity peaked at stimulation with 5 mM ammonia treatment at about 20 nmol/min/10⁶ cells, and levels were observed higher than those seen using primary human hepatocytes. For Protocol A, ureagenesis activity peaked at stimulation with 10 mM ammonia at a level similar to primary human hepatocytes, about 3 nmol/min/10⁶ cells.

This example demonstrates that forskolin treatment during maturation of hepatocyte-like cells can induce urea cycle genes and drive ureagenesis and ammonia detoxification activity.

### Example 2. 3D Hepatocyte Differentiation Method Including Forskolin

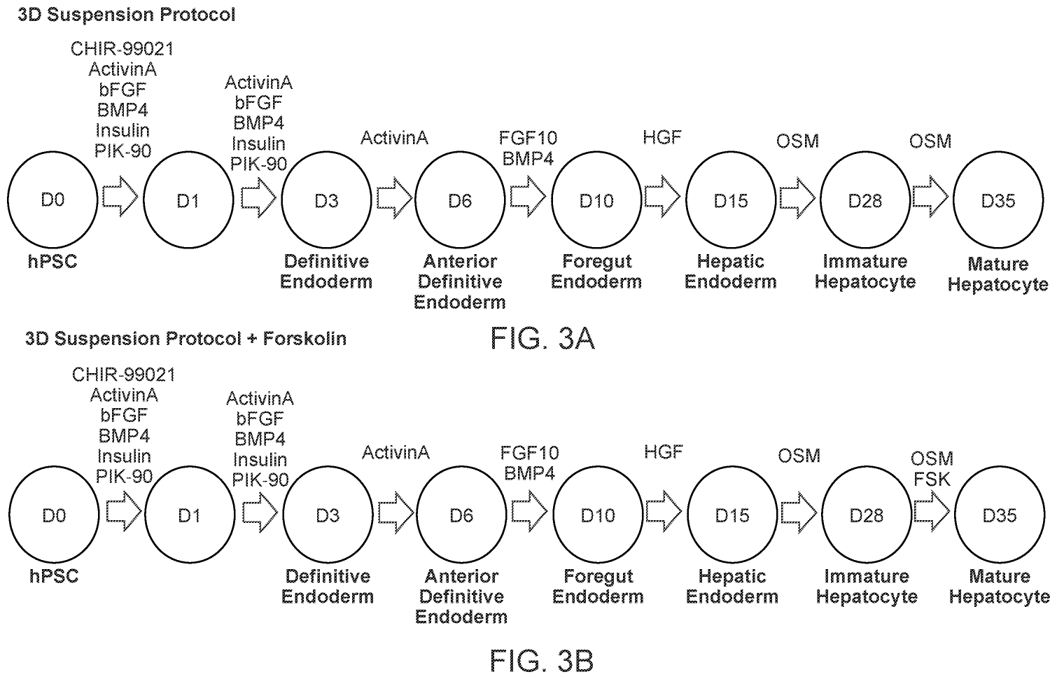

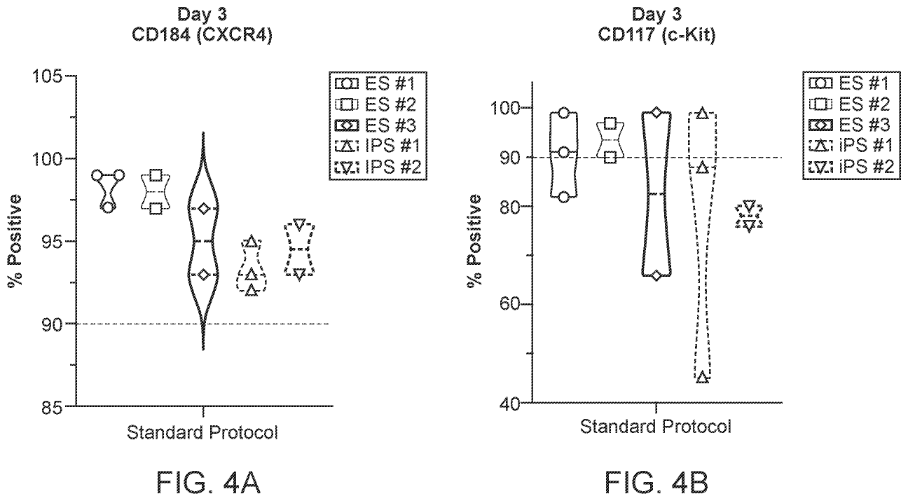

This example shows a 3D differentiation protocol that includes the use of forskolin in the process of differentiating pluripotent stem cells to mature hepatocytes. FIG. 3A shows a diagram of a 3D differentiation protocol without forskolin (FIG. 3A) and the a 3D differentiation protocol that includes the addition of forskolin (FIG. 3B) during maturation from immature hepatocyte to mature hepatocyte. In the process, the pluripotent stem cells differentiate through different stages including a definitive endoderm stage, an anterior definitive endoderm stage, a foregut endoderm stage, a hepatic endoderm stage, an immature hepatocyte stage, and a mature hepatocyte stage. Flow cytometry analysis showed expression of canonical definitive endoderm markers CD184 (CXCR4) and CD117 (c-kit) in cells at day 3 of the a 3D differentiation process (FIGS. 4A and 4B). All pluripotent cell lines robustly differentiated in the endoderm lineage in the 3D format, with all cell lines achieving a target efficiency of 90% CXCR4 expressing cells.

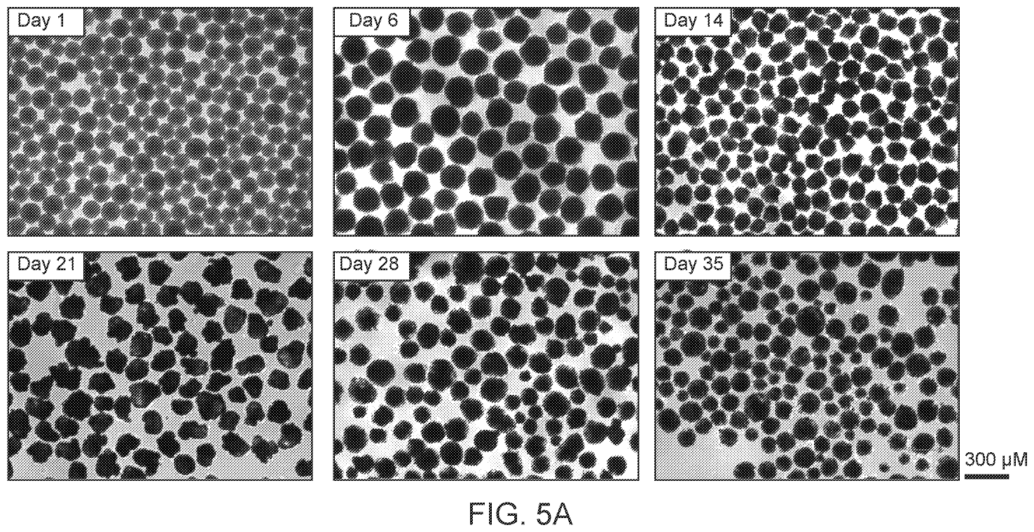

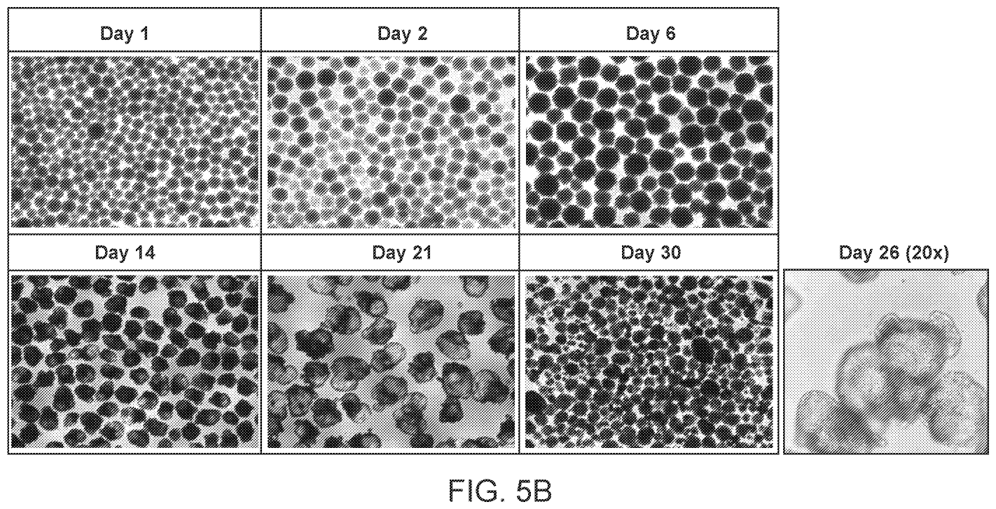

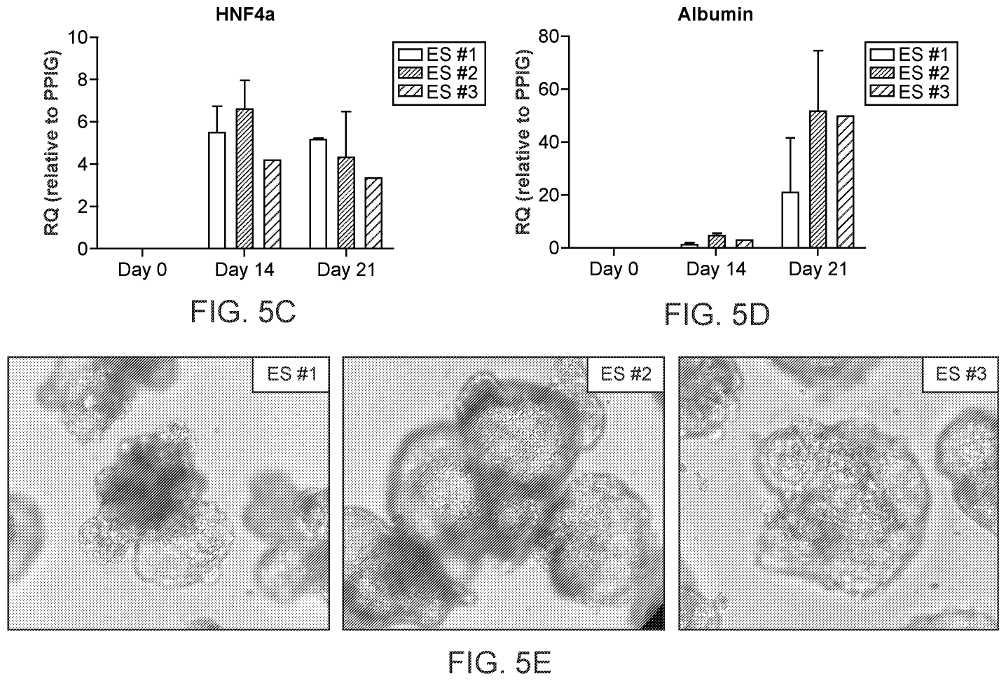

The pluripotent stem cells were differentiated in suspension aggregation culture in order to mimic the native substrate stiffness that occurs in a native embryo. Over the course of the differentiation, the aggregate size remained stable and consistent, with mean aggregate diameter between 150-300 μM. Images of suspension aggregation cultures at different stages of the differentiation process are shown in FIGS. 5A-5B. The differentiating cells at day 14 and day 21 expressed hepatocyte-specific markers such as HNFa and albumin (FIGS. 5C and 5D). Phase contrast images of the spheroids from the 3D cultures at day 14 are shown in FIG. 5E. The spheroid clusters contained multi-lobular structures containing cuboidal hepatocyte-like cells.

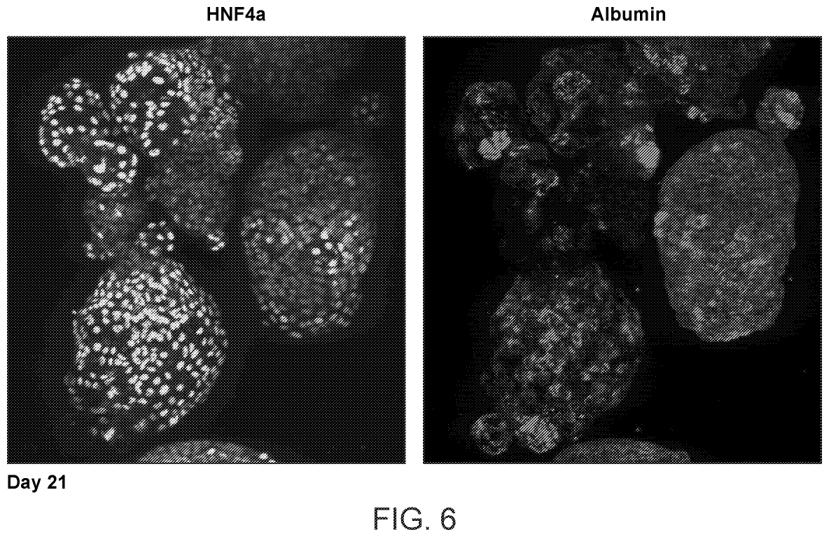

Day 21 hepatic spheroids were fixed and immunostained in whole mount for HNF4a and albumin, markers of hepatocyte differentiation. Robust expression of both markers was detected in each hepatic spheroid, consistent with a high differentiation efficiency in the culture (FIG. 6). Whole mount immunofluorescence staining of the spheroids also showed expression of HNFa and albumin (FIG. 6).

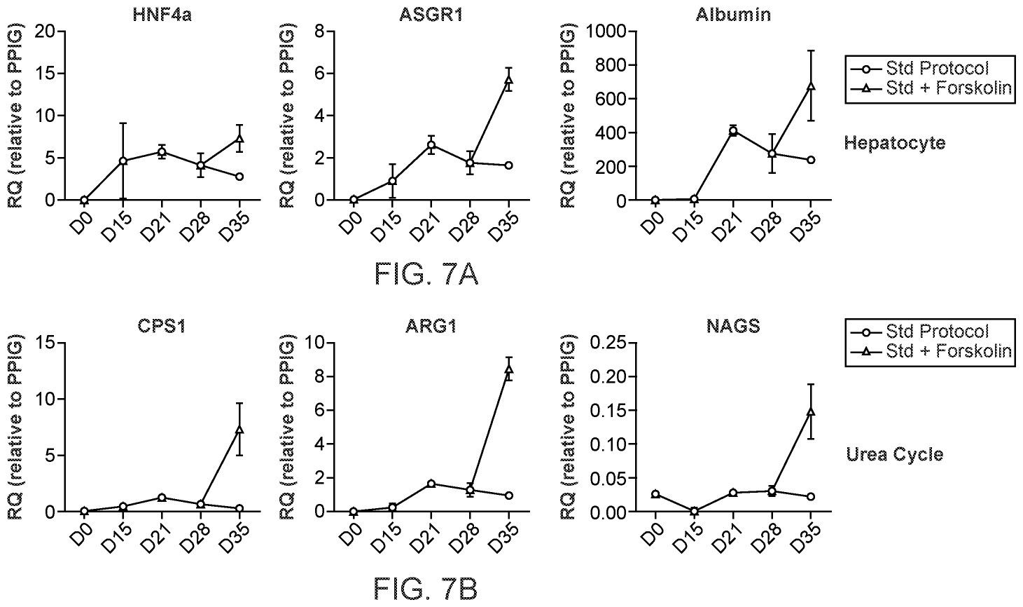

Gene expression analysis was performed on hepatocyte-like cells produced by either (i) a 3D differentiation protocol or (ii) a 3D differentiation protocol with the addition of 10 μM forskolin from days 28 to day 35. Duplicate RNA samples were isolated at selected time point of the differentiation and gene expression was analyzed by Taqman qPCR. Gene expression analysis of hepatocyte maturation genes (FIG. 7A) and urea cycle genes (FIG. 7B) showed increases in expression in differentiating cells treated with forskolin compared to those untreated.

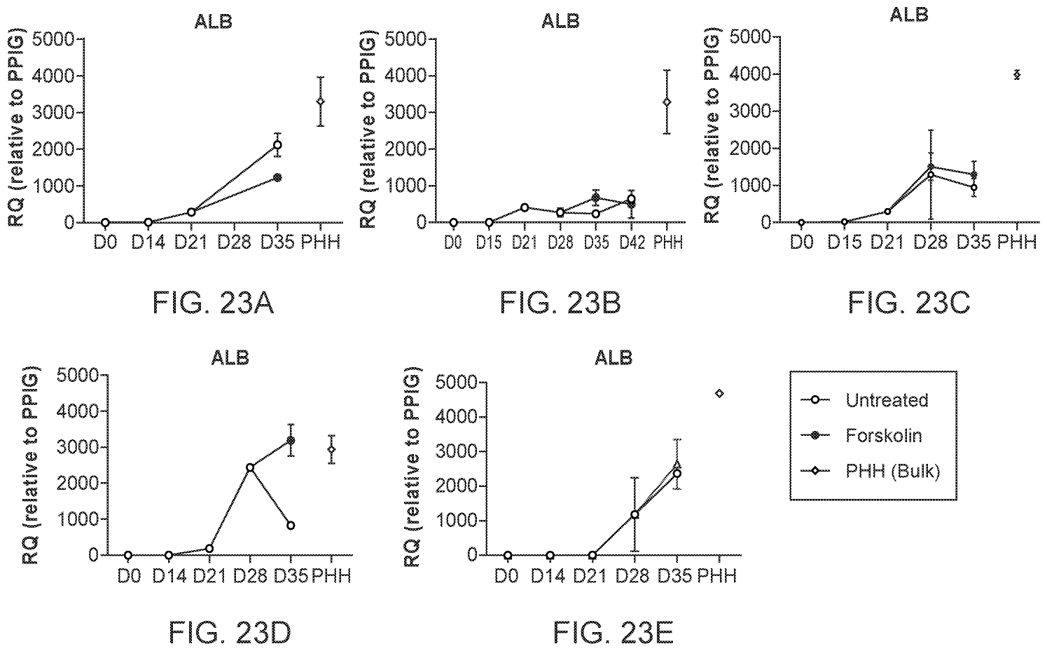

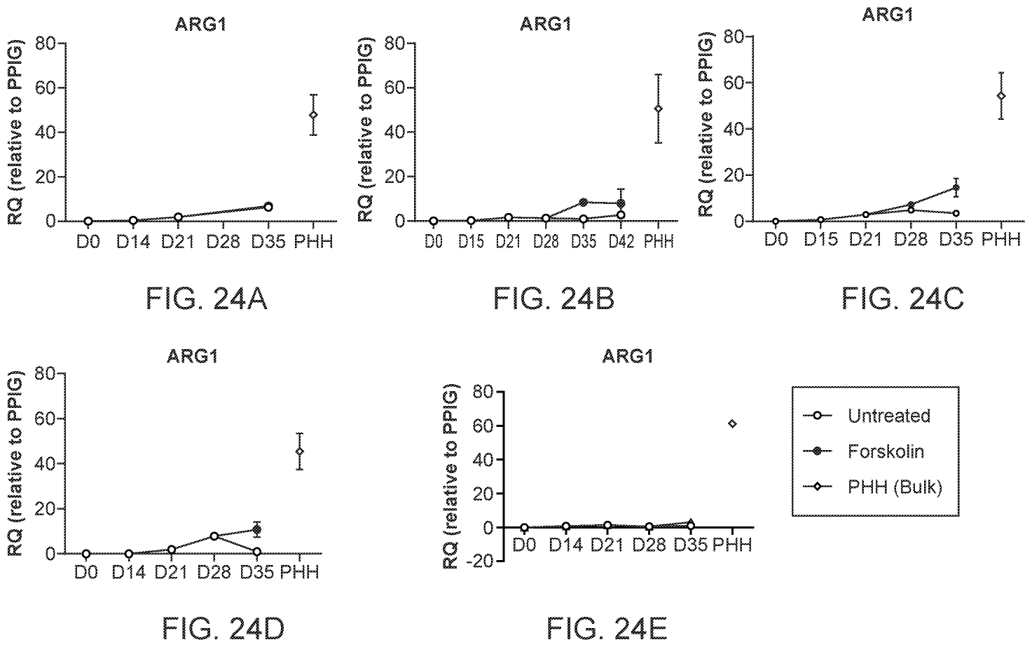

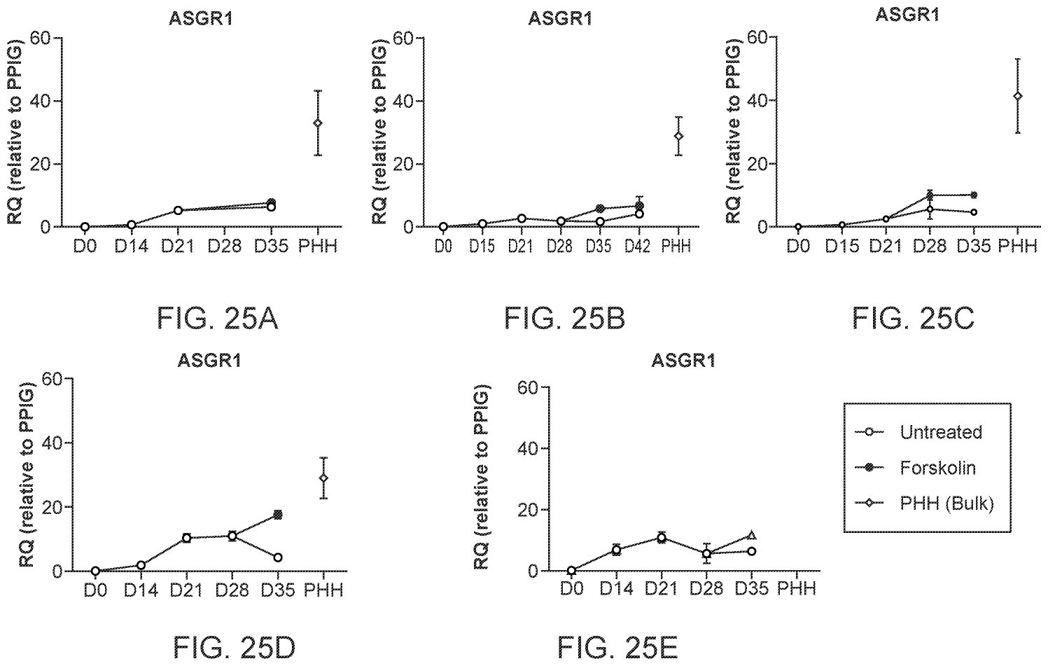

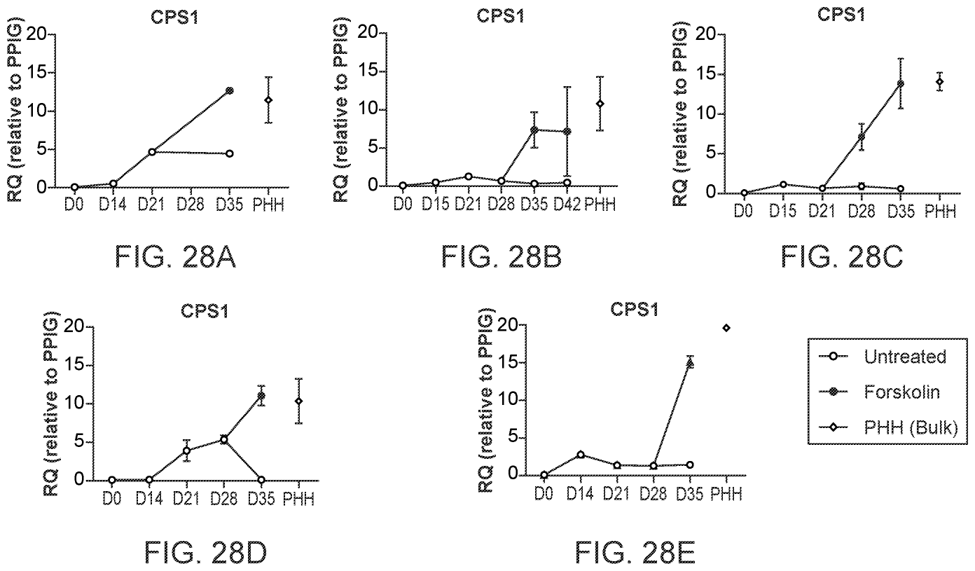

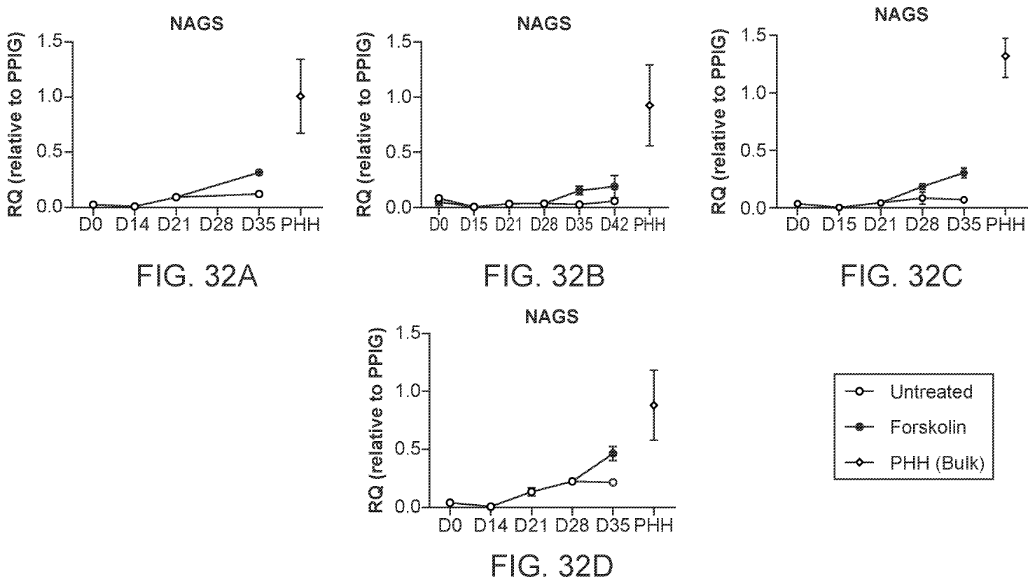

Consistent with data seen in 2D hepatocyte differentiations, addition of forskolin during the last 7 days of the differentiation increase the expression of general hepatocyte maturation markers such as ASGR1 (FIGS. 25D-E), and albumin (FIG. 23D-E), as well as increasing the expression of the urea cycle genes CPS1 (FIGS. 28D-E), ARG1 (FIGS. 24D-E), and NAGS (FIG. 32D).

This example describes an effective method for the differentiation of pluripotent stem cells into mature hepatocytes. The 3D differentiation method includes culturing differentiating cells as spheroid and treating them with forskolin as the cells become mature hepatocytes.

### Example 3. Substrate Stiffness on the Maturation of Hepatocyte-Like Cells

This example shows that albumin production rate of hepatocyte-like cells increases with stiffness of PEG hydrogels in 2D differentiation methods.

A 2D differentiation protocol was followed for the first 14 days of differentiation (Protocol A). After which cells were seeded onto 2D poly(ethylene glycol) (PEG) hydrogels (PEG solution) and cultured until day 35. PEG hydrogels of differing elastic modulus were tested. A PEG hydrogel with 2.5% solid content was referred to as a “soft” PEG hydrogel. A PEG hydrogel with 10% solid content was referred to as a “stiff” PEG hydrogel. The soft PEG hydrogel exhibited an elastic modulus of greater than 1 kPa. The stiff PEG hydrogel exhibited an elastic modulus of about 8 kPa. Albumin production by the differentiated hepatocytes was determined using known assays including CYP3A4 Promega Glo and Albumin ELISA assays.

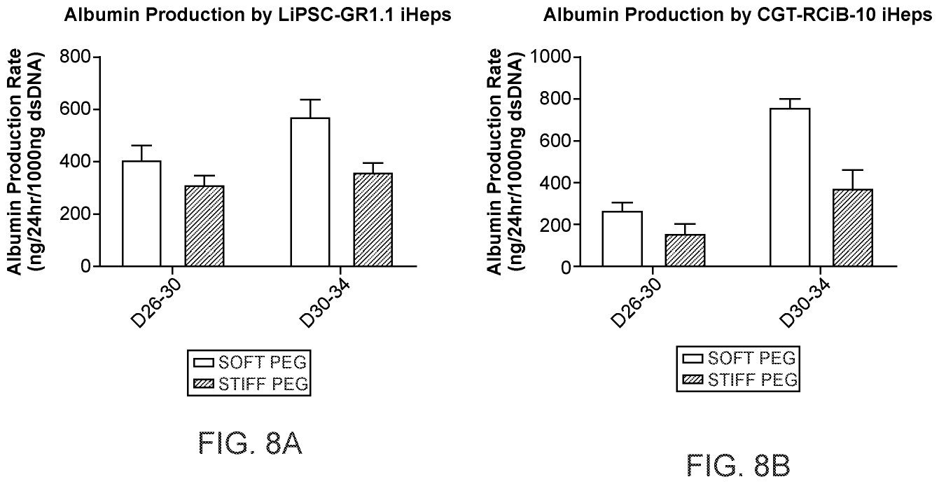

FIGS. 8A and 8B shows the albumin production rate of hepatocyte-like cells differentiated from two iPSC cell lines. The results show that albumin production by mature hepatocyte-like cells increased when the hepatocyte cells were differentiated using a substrate with a low stiffness index (such as a low elastic modulus) compared to a high stiffness index.

### Example 4. Vitamin K on the Maturation of Hepatocyte-Like Cells

This example shows that differentiation in the presence of vitamin K produces mature hepatocyte-like cells with increased urea synthesis.

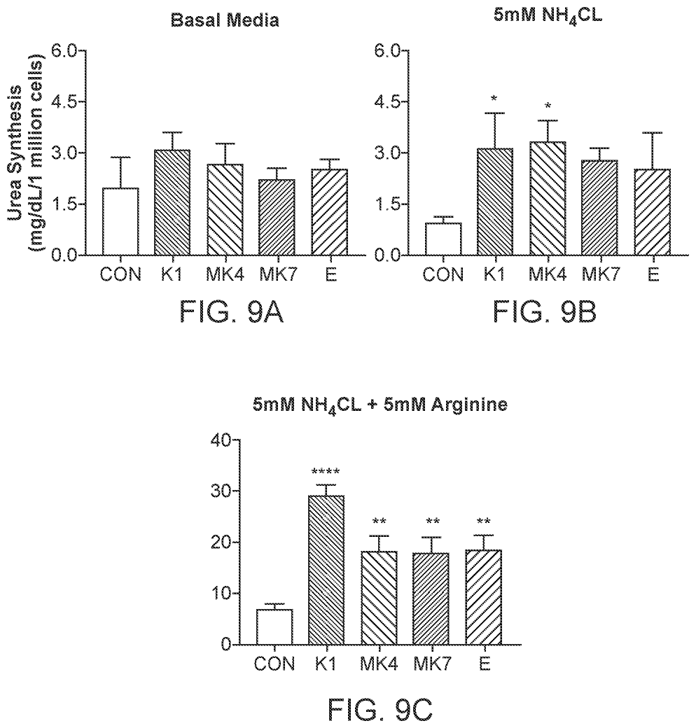

Human pluripotent stem cell-derived hepatocytes (hPSC-Heps) were obtained after 21 days of differentiation using a 2D adherent protocol. At day 21 the hPSC-Heps were aggregated into spheroids, and then encapsulated within alginate 48 hours later. The spheroids including the alginate encapsulated spheroids were cultured for 14 days in either vitamin K1, vitamin K2 MK4, vitamin K1 MK7, vitamin E, or a combination of all. The spheroids were cultured at a concentration of about 1×10⁶ encapsulated cells/ml for the culture period. Urea synthesis was assayed and the results are shows in FIGS. 9A-9C.

Culturing the spheroids in media containing either vitamin K1 or vitamin K2 (vitamin K2 MK4 or MK7) increased the functional maturation status of the hepatocytes (FIGS. 9A-9C). The vitamin K1- and vitamin K2-treated mature hepatocyte-like cells were more effective at detoxifying ammonia (FIG. 9B). In an urea cycle assay supplemented with excess arginine (a urea cycle substrate), the vitamin K1- and vitamin K2-treated mature hepatocyte-like cells detoxified ammonia even faster (FIG. 9C). The results support the notion that vitamin K1 and/or vitamin K2 supplementation can facilitate the in vitro generation of mature hepatocyte-like cells possessing ureagenesis activity.

### Example 5. Removal of Hepatocyte Growth Factor (HGF) for the Maturation of Hepatocyte-Like Cells

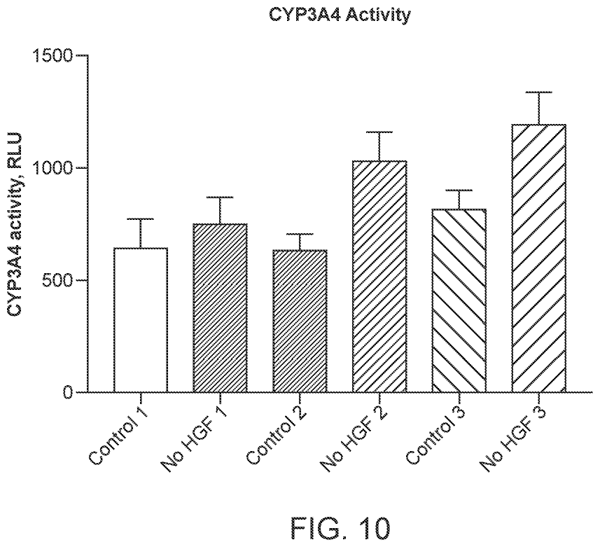

This example shows that differentiation in media that is free of HGF increases maturation of immature hepatocyte-like cells to mature hepatocyte-like cells. Mature hepatocyte-like cells were differentiated from human pluripotent stem cells using a 2D differentiation protocol for the first 14 days of differentiation, and then the cells were cultured in media lacking recombinant HGF. At day 14, hepatocytes at the hepatic endoderm stage were dissociated and seeded into these plates in fully supplemented HepatoZYME-SFM media. 24 hours later the cells were cultured and maintained in media lacking recombinant HGF. Hepatocyte maturation was determined using a CYP3A4 enzyme activity assay. The absence of HGF in the culture media after the first 14 days of differentiation enhanced hepatocyte maturation (FIG. 10).

Differentiation using media free of HGF also increased maturation of immature hepatocyte-like cells to mature hepatocyte-like cells in 3D differentiation methods. Briefly, 3D spheroid cultures were produced using a 2D differentiation protocol for the first 14 days of differentiation, and then the cells were cultured in media lacking recombinant HGF. In day 21, the differentiating hepatocytes were aggregated into spheroids, and then encapsulated within alginate 48 hours later. Cells cultured in media without HGF were maintained until day 22 Control cells were cultured in media containing HGF. Hepatocyte maturation was determined using a CYP3A4 enzyme activity assay (data not shown).

### Example 6. Forskolin of the Maturation of Hepatocyte-Like Cells

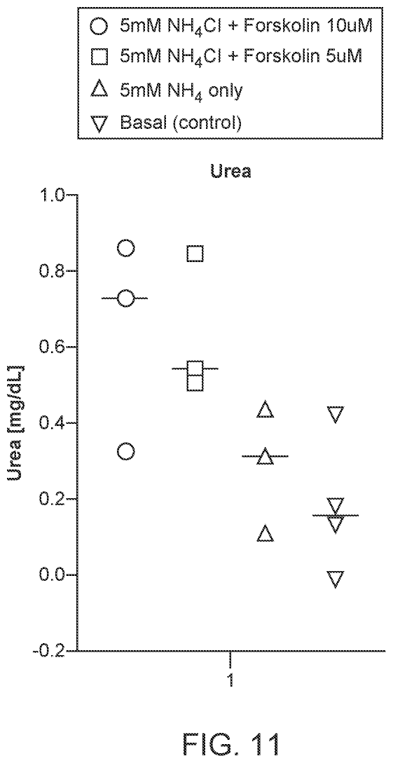

This example shows that differentiation media supplemented with forskolin increases maturation of immature hepatocyte-like cells to mature hepatocyte-like cells. Mature hepatocyte-like cells were differentiated from human pluripotent stem cells using a 2D differentiation protocol for the first 26 days of differentiation, and then the immature hepatocyte-like cells were cultured in media containing forskolin (ranging from about 5 μM to about 10 μM). At day 35, the activity for the hepatocytes was determined using a urea production assay. The presence of forskolin in the culture media after the first 26 days of differentiation enhanced the ureagenesis activity of the differentiated mature hepatocyte (FIG. 11). The increase in urea production appeared to be dose dependent.

### Example 7. Oxygen Tension of the Maturation of Hepatocyte-Like Cells

This example shows the effect of oxygen conditions on the maturation of immature hepatocyte-like cells to mature hepatocyte-like cells. Mature hepatocyte-like cells were differentiated from human pluripotent stem cells using either a 2D differentiation protocol or a 3D differentiation protocol (see, for example, Hannan et al., supra and Blackford et al., supra) with the modifications below.

In the modified 2D differentiation protocol, at day 17 the differentiating cells were plated on collagen type I coated plates and cultured in either a hypoxic condition (5% oxygen content) or a normoxic condition (atmospheric oxygen content).

In the modified 3D differentiation protocol, at day 17 the differentiating cells were aggregated into spheroids, and then encapsulated in alginate 48 hours later. The encapsulated spheroid were cultured in either a hypoxic condition (5% oxygen content) or a normoxic condition (atmospheric oxygen content).

The ureagenesis activity of the mature hepatocyte-like cells generated using either of differentiation protocols was determined. The results from the urea assay show that culturing cells under normoxic conditions significantly increased ureagenesis of mature hepatocyte-like cells cultured using a 3D differentiation method (FIG. 12).

### Example 8. 3D Clustering and Alginate Encapsulation to Produce Mature Hepatocyte-Like Cells

This example describes a method for producing mature hepatocyte-like cells using 3D clustering and alginate encapsulation. The example also describes urea production activity and urea cycle gene expression of these mature hepatocyte-like cells. In addition, the activity of encapsulated hepatocyte-like cells that were transplanted into a mouse model of acute liver failure was described.

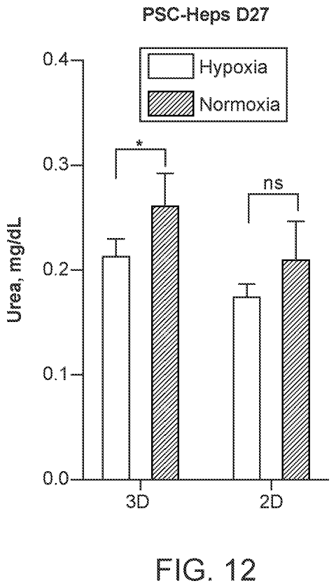

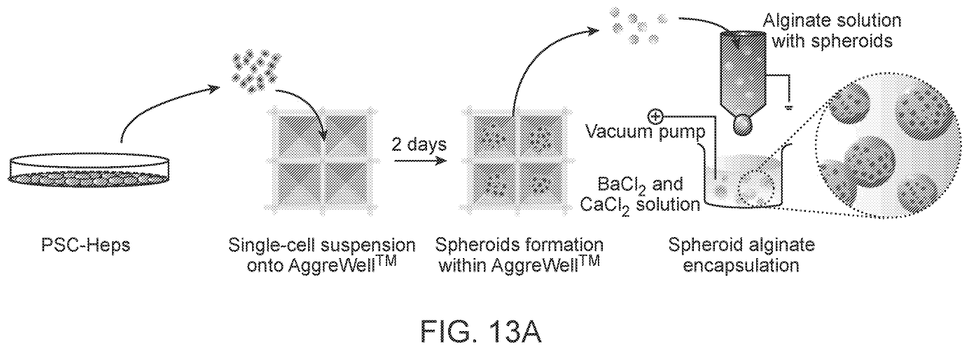

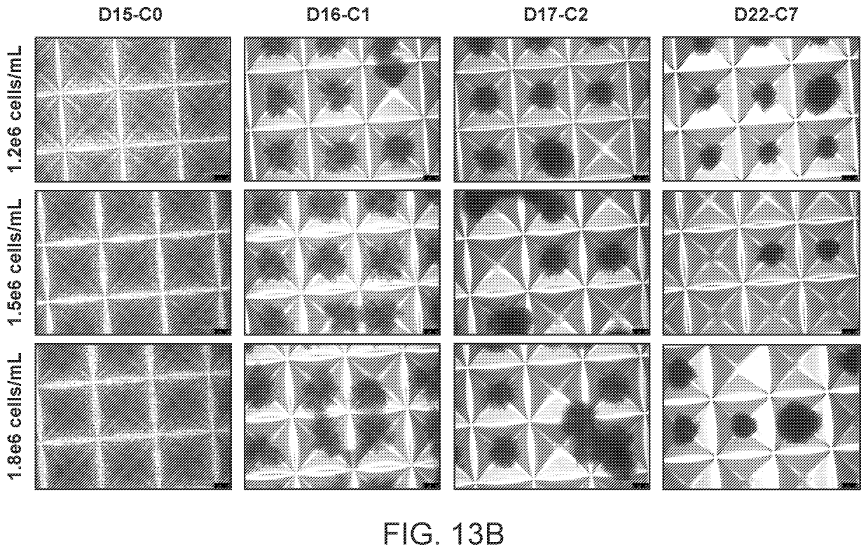

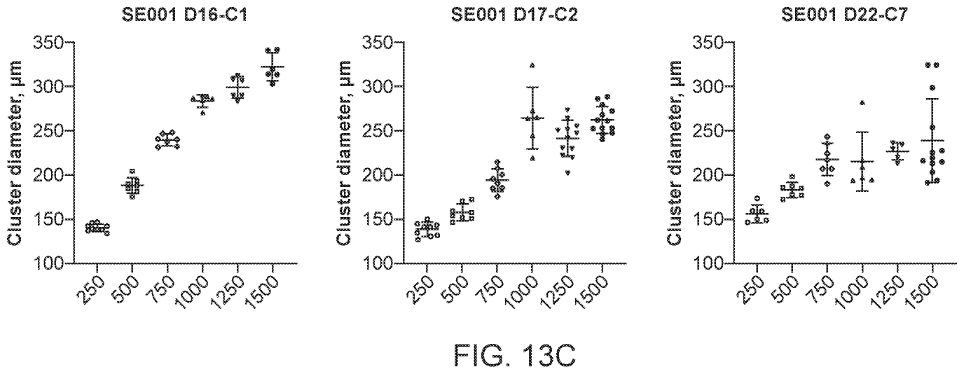

FIG. 13A depicts a schematic diagram of the 3D differentiation method starting with immature hepatocytes in a 2D culture. The cells were dissociated into a single-cell suspension and then aggregated into spheroids. Next, the spheroid were encapsulated in alginate. FIG. 13B shows aggregation of the immature hepatocyte-like cells into spheroids at different days of differentiation (d15, d16, d17 and d22). Three concentrations of cells (1.2×10⁶/ml, 1.5×10⁶/ml, and 1.8×10⁶/ml) were evaluated. FIG. 13C shows that aggregates (clusters) containing about 750 yielded clusters of significant size with negligible necrosis. Alginate encapsulation did not affect hepatocyte function. It appears that 3D clustering prior to alginate encapsulation is important for cell survival.

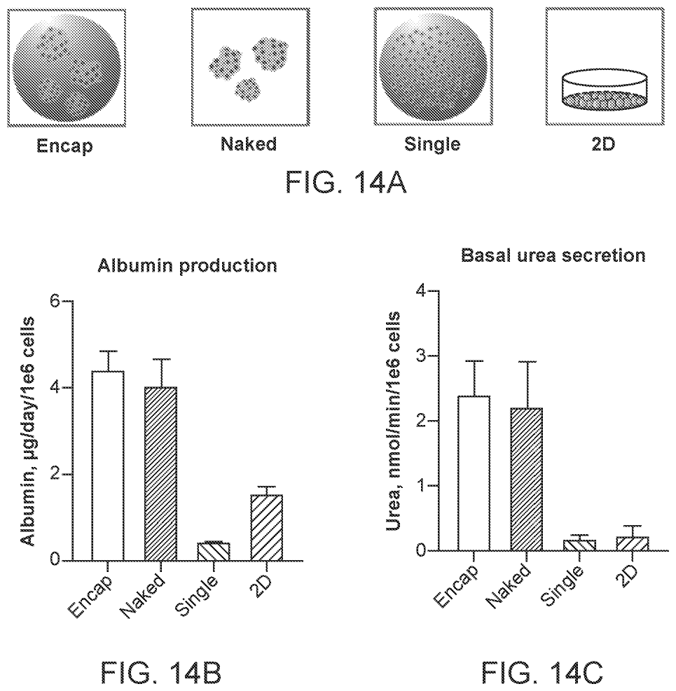

The mature hepatocyte-like cells produced from alginate encapsulated spheroids were compared to cells generated from several other methods including naked (non-encapsulated) spheroids, alginate encapsulated single-cell suspension, and 2D differentiation (FIG. 14A).

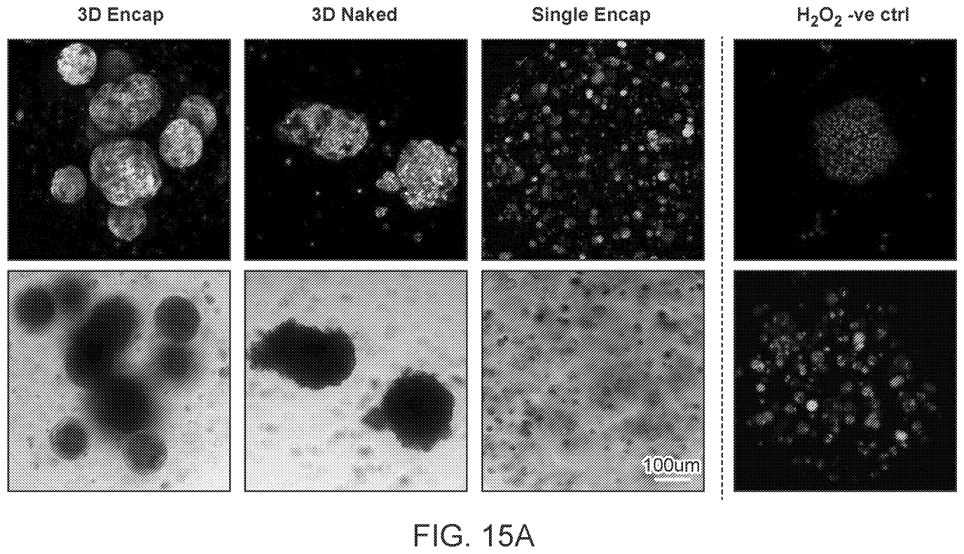

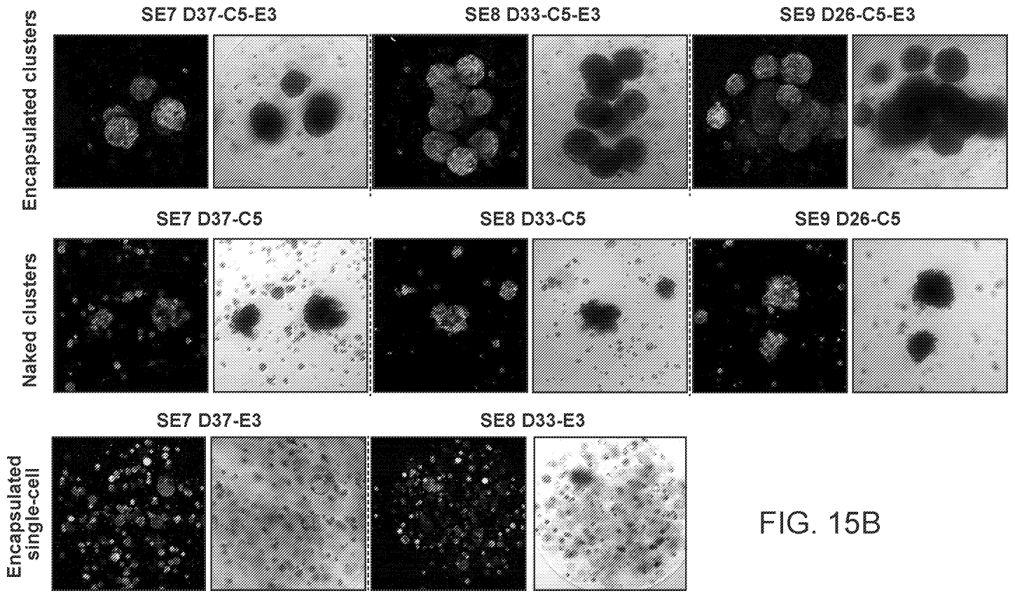

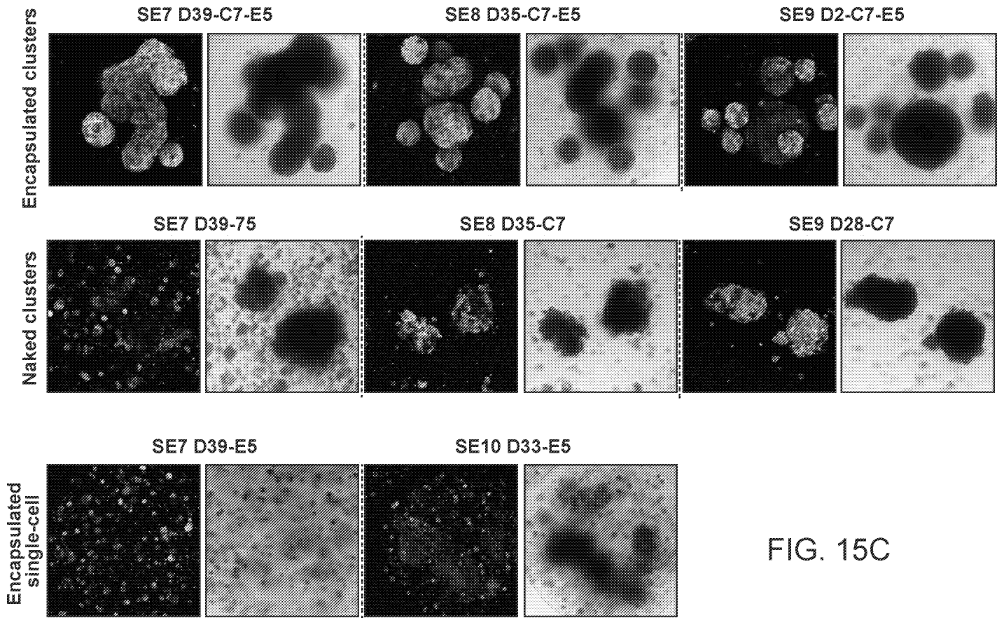

FIG. 14A shows a schematic diagram of various differentiation methods that include forming: (1) alginate encapsulated spheroids, (2) naked spheroids, (3) alginate encapsulated single-cell suspensions, or (4) 2D cell layers. FIG. 14B shows albumin production in hepatocyte-like cells produced using the formats of FIG. 14A. FIG. 14C shows basal urea secretion in these hepatocyte-like cells. Images of hepatocyte cells in the alginate encapsulated spheroids, naked spheroids and alginate encapsulated single-cell suspension are shown in FIG. 15A (7 days post encapsulation), FIG. 15B (3 days post encapsulation), and FIG. 15C (5 days post encapsulation). The 3D cluster configuration maintained cell viability better than single-cell suspensions in alginate beads. Naked 3D clusters were prone to either disintegration or fusion, thus leading to higher cell death.

Urea production was analyzed in the mature hepatocyte-like cells formed by the methods described above.

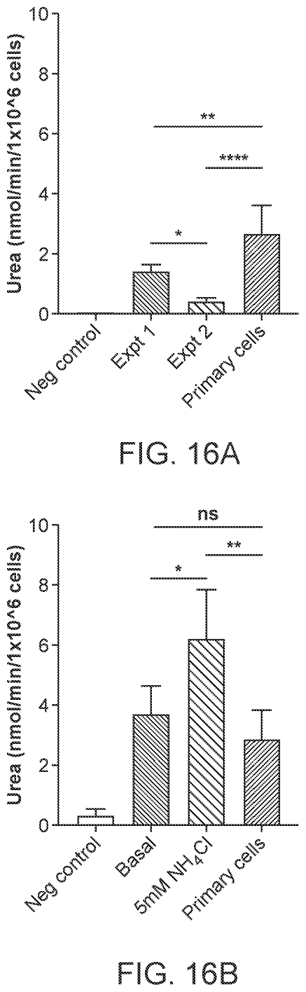

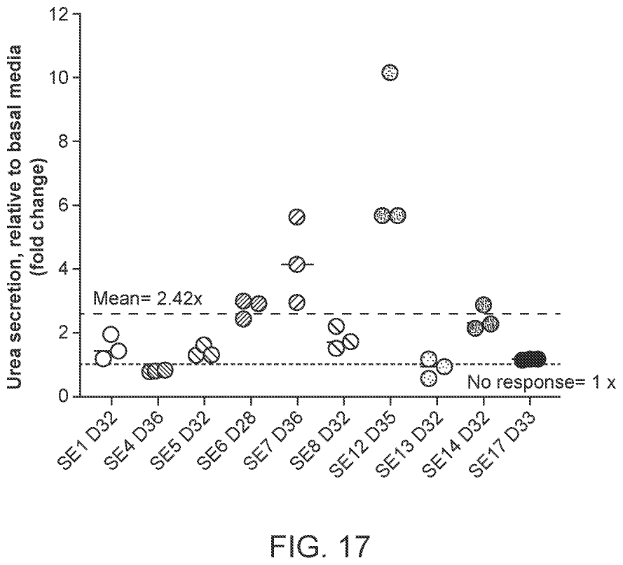

Primary hepatocytes produced more urea compared to hepatocytes cultured using the 2D differentiation method (FIG. 16A). Urea assays were performed on day 21 hepatocytes from the 2D culture. Hepatocytes produced by using the 3D differentiation method including forming alginate encapsulated spheroids were more effective at urea production compared to primary hepatocytes (FIG. 16B). In a further experiment, hepatocytes from the alginate encapsulated spheroids were more effective at ammonia-urea conversion when exposed to ammonia compared to basal media (FIG. 17).

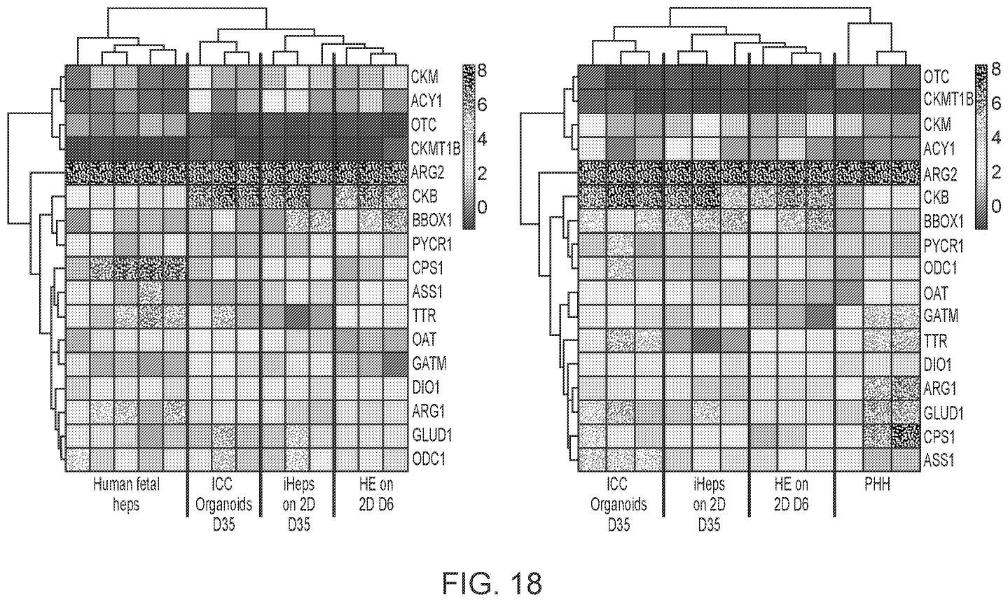

RNAseq analysis was performed to compare gene expression of urea cycle genes in samples from human fetal hepatocytes, day 35 hepatocytes differentiated using a 3D differentiation method, day 6 and day 35 hepatocytes differentiated using a 2D differentiation method, and primary human hepatocytes (PHH). FIG. 18 shows heat maps and hierarchal clustering of urea cycle gene in the samples.

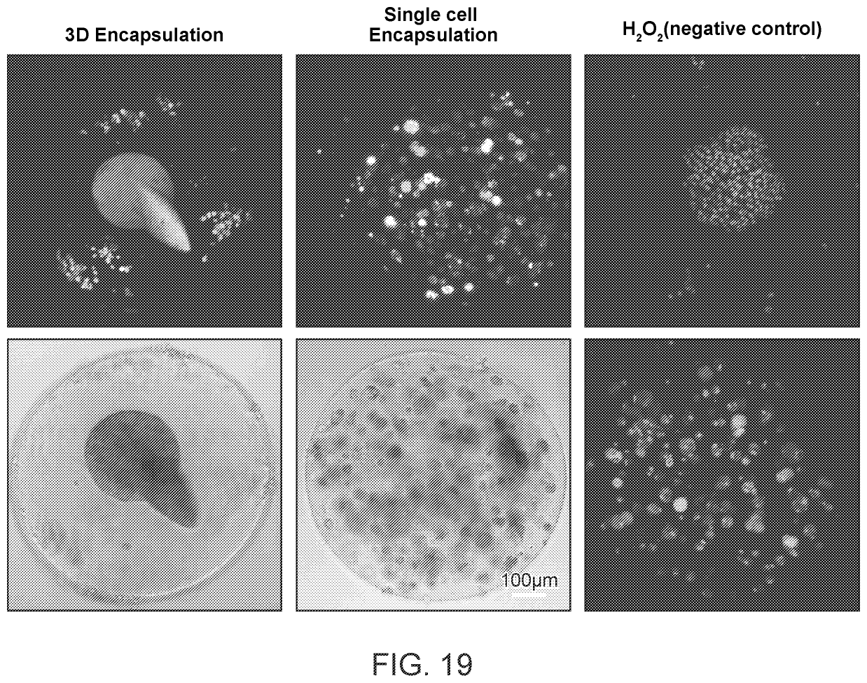

To test the in vivo cell survival of the mature hepatocytes differentiated from human pluripotent stem cells, the alginate encapsulated spheroids were transplanted into the liver of a mouse model. Images of the alginate encapsulated hepatocyte spheroids and encapsulated single-cell suspensions are shown in FIG. 19.

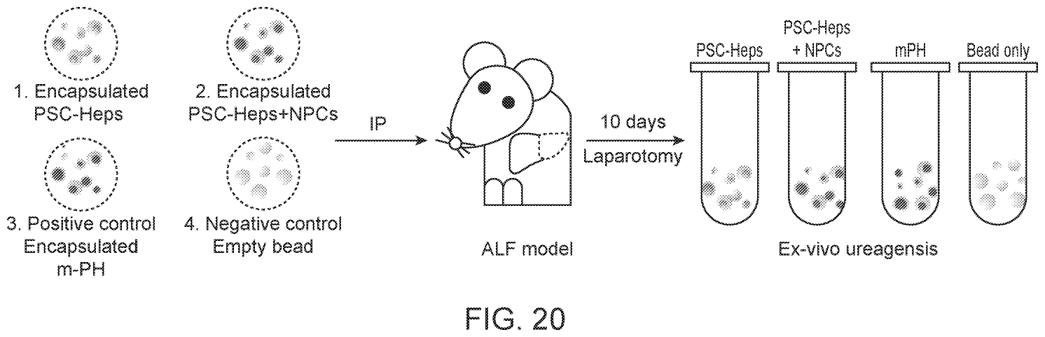

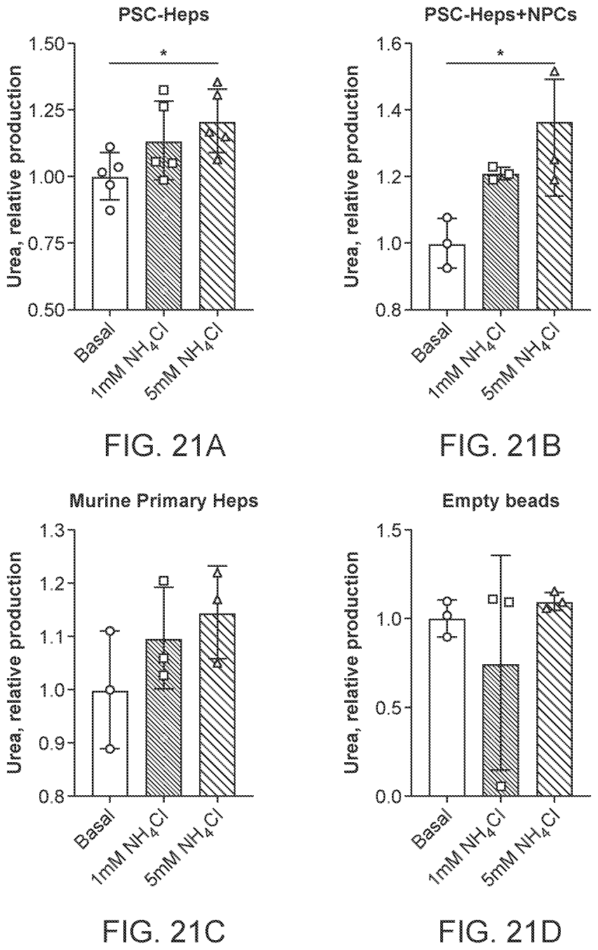

Additional studies for analyzing ureagenesis of the differentiated hepatocytes transplanted into a mouse model of acute liver failure (ALF) were performed (FIG. 20). Alginate encapsulated spheroids containing mature hepatocytes or alginate encapsulated spheroids containing mature hepatocytes and non-parenchymal liver cells (NPCs) were transplanted into an ALF mouse model. Day 10 post transplantation, the ells were harvested by laparotomy and the recovered cells were analyzed for ex vivo ureagenesis. As a positive control, encapsulated mouse primary hepatocytes were transplanted into an ALF mouse model, and as a negative control empty beads were transplanted into an ALF mouse model. FIGS. 21A-21D show ex vivo urea production in the hepatocytes exposed to basal media and increasing amounts of ammonia. Ammonia conversion to urea was detected in the encapsulated hepatocytes and the encapsulated cell mixture of hepatocytes and NPCs.

The results show that mature hepatocyte-like cells generated using a 3D differentiation method of the present technology were effectively transplanted into a mouse model and the mature hepatocyte-like cells exhibited urea production activity.

### Example 9. Urea Cycle Gene Expression of Hepatocyte-Like Cells Treated with Forskolin and Produced Using 3D and 2D Differentiation Methods

This example provides urea cycle gene expression of hepatocyte-like cells differentiated using three different methods, such as a 3D differentiation method with forskolin treatment at days 21-35 (method A), a 3D differentiation method with forskolin treatment at days 30-35 (method B), and a 2D differentiation method with forskolin treatment at days 28-35 (method C). The expression of the following genes were measured: alpha fetoprotein (AFP), albumin (ALB), arginase 1 (ARG1), asialoglycoprotein receptor 1 (ASGR1), agininosuccinate lyase (ASL), argininosuccinate synthase 1 (ASS1), carbamoyl-phosphate synthase 1 (CPS1), glucose-6-phosphatase (G6PC), hepatocyte nuclear factor 4 alpha (HNF4a), keratin type I cytoskeletal 18 (KRT18), N-acetylgalactosamine (NAGS), ornithine transcarbamylase (OTC), and SOX9. Expression of cardiac-specific markers NKX2.5 and TNNT2 were used as a negative expression control.

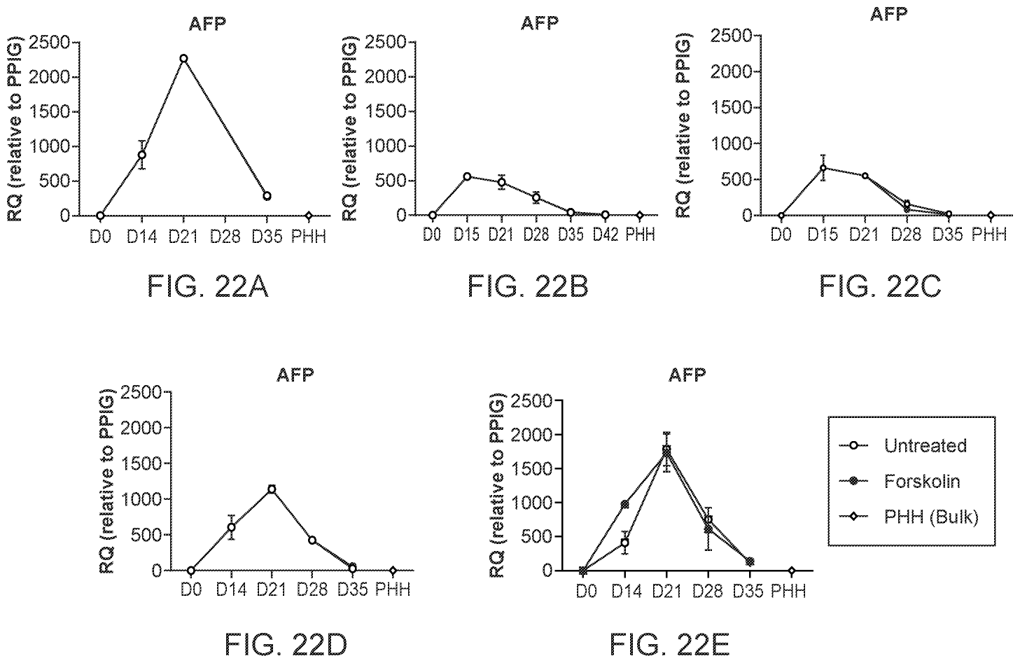

FIGS. 22A-22E show AFP expression in hepatocytes differentiating from pluripotent stem cells at various days of the differentiation method A (FIGS. 22A and 22C), the differentiation method B (FIG. 22B) and the differentiation method C (FIGS. 22D and 22E). AFP is a marker for immature hepatocytes.

FIGS. 23A-23E show ALB expression in hepatocytes differentiating from pluripotent stem cells at various days of the differentiation method A (FIGS. 23A and 23C), the differentiation method B (FIG. 23B), and the differentiation method C (FIGS. 23D and 23E). ALB is a marker for mature hepatocytes.

FIGS. 24A-24E show ARG1 expression in hepatocytes differentiating from pluripotent stem cells at various days of the differentiation method A (FIGS. 24A and 24C), the differentiation method B (FIG. 24B), and the differentiation method C (FIGS. 24D and 24E). ARG1 is a urea cycle enzyme.

FIGS. 25A-25E show ASGR1 expression in hepatocytes differentiating from pluripotent stem cells at various days of the differentiation method A (FIGS. 25A and 25C), the differentiation method B (FIG. 25B) and the differentiation method C (FIGS. 25D and 25E). ALB is a mature hepatocyte surface marker.

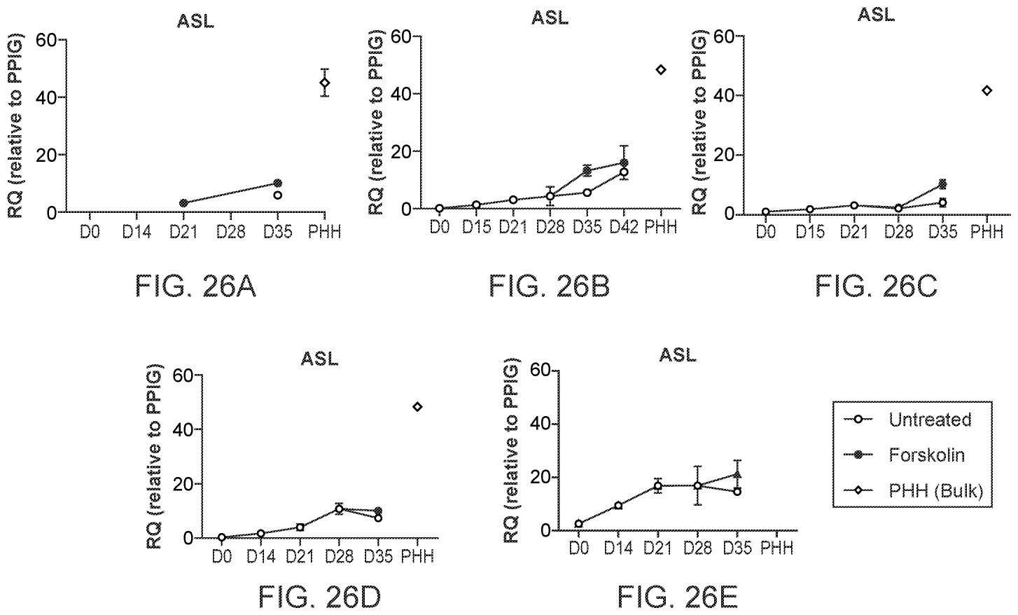

FIGS. 26A-26E show ASL1 expression in hepatocytes differentiating from pluripotent stem cells at various days of the differentiation method A (FIGS. 26A and 26C), the differentiation method B (FIG. 26B), and the differentiation method C (FIGS. 26D and 26E). ASL is a urea cycle enzyme.

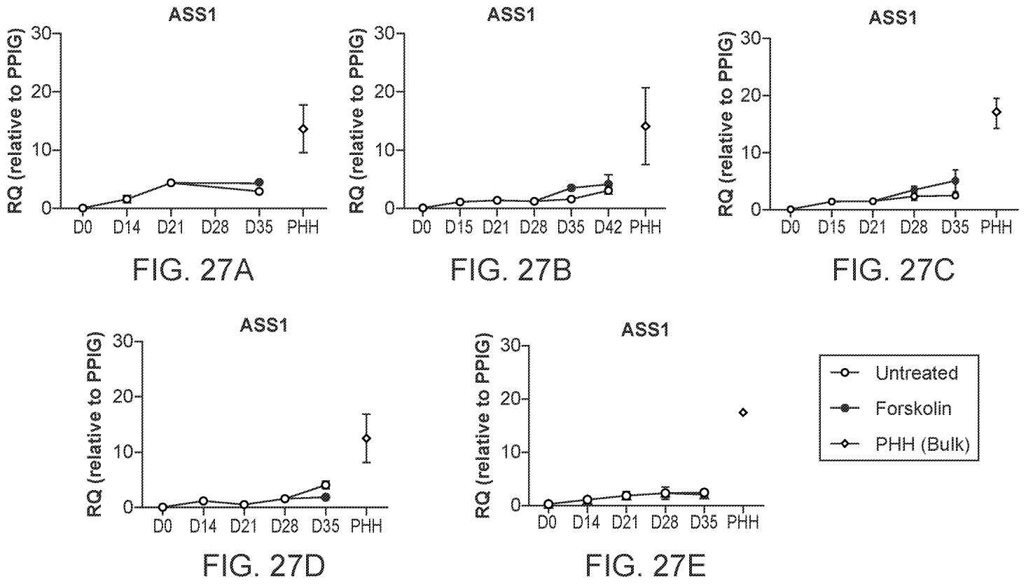

FIGS. 27A-27E show ASS1 expression in hepatocytes differentiating from pluripotent stem cells at various days of the differentiation method A (FIGS. 27A and 27C), the differentiation method B (FIG. 27B), and the differentiation method C (FIGS. 27D and 27E). ASS1 is a urea cycle enzyme.

FIGS. 28A-28E show CPS1 expression in hepatocytes differentiating from pluripotent stem cells at various days of the differentiation method A (FIGS. 28A and 28C), the differentiation method B (FIG. 28B), and the differentiation method C (FIGS. 28D and 28E). CPS1 is a urea cycle enzyme.

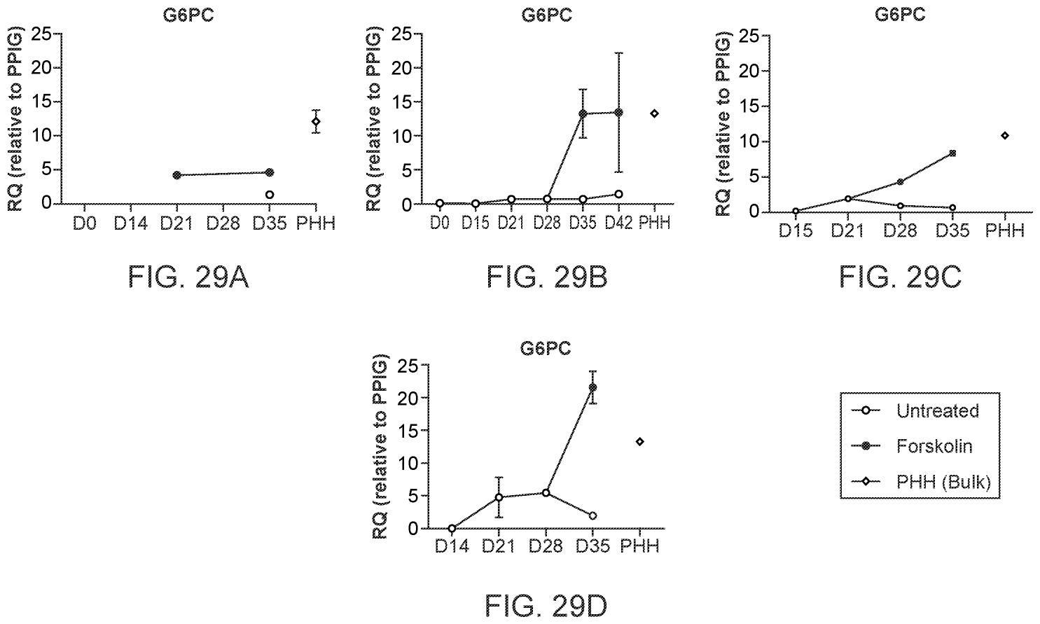

Hepatocytes differentiated using either the 3D or 2D methods expressed G6PC, a hepatocyte identity marker. FIGS. 29A-29D show G6PC expression in hepatocytes differentiating from pluripotent stem cells at various days of the differentiation method A (FIGS. 29A and 29C), the differentiation method B (FIG. 29B), and the differentiation method C (FIG. 29D). G6PC is a marker for cAMP activation.

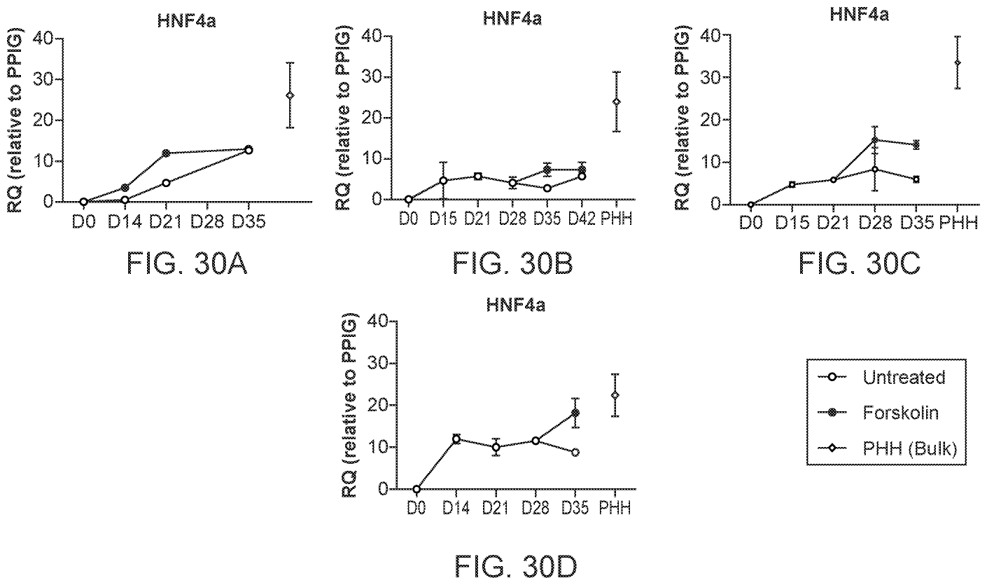

Hepatocytes differentiated using either the 3D or 2D methods expressed HNFa, a hepatocyte identity marker. FIGS. 30A-30D show HNFa expression in hepatocytes differentiating from pluripotent stem cells at various days of the differentiation method A (FIGS. 30A and 30C), the differentiation method B (FIG. 30B), and the differentiation method C (FIG. 30D). HNFa is a hepatocyte identity marker.

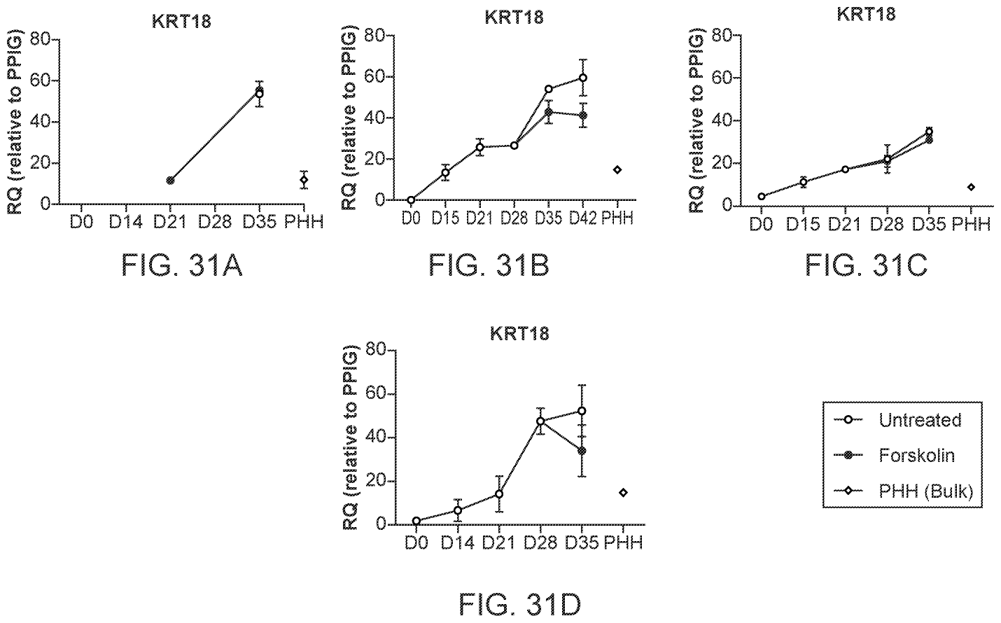

Hepatocytes differentiated using either the 3D or 2D methods expressed KRT18, a hepatocyte identity marker. FIGS. 31A-31D show KRT18 expression in hepatocytes differentiating from pluripotent stem cells at various days of the differentiation method A (FIGS. 31A and 31C), the differentiation method B (FIG. 31B), and the differentiation method C (FIG. 31D). KRT18 is a hepatocyte identity marker.

Hepatocytes differentiated using either the 3D or 2D methods of the present technology expressed NAGS, a urea cycle enzyme. FIGS. 32A-32D show NAGS expression in hepatocytes differentiating from pluripotent stem cells at various days of the differentiation method A (FIGS. 32A and 32C), the differentiation method B (FIG. 32B), and the differentiation method C (FIG. 32D). NAGS is a urea cycle enzyme.

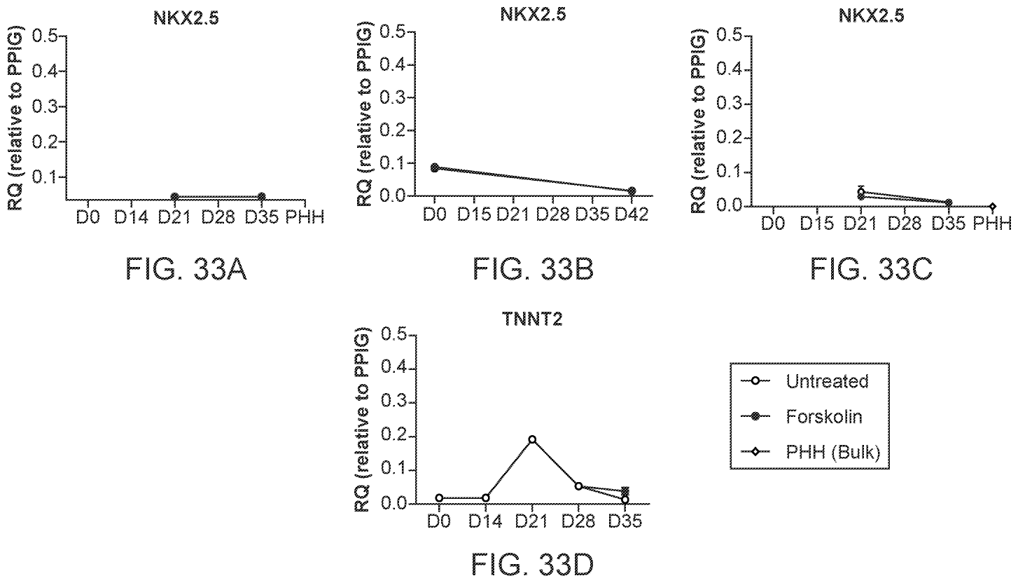

The differentiated cells produced using the 3D or 2D methods did not express cardiac-specific markers such as NKX2.5 and TNNT2. FIGS. 33A-33C show NKX2.5 expression in hepatocytes differentiating from pluripotent stem cells at various days of the differentiation method A (FIGS. 33A and 33C) and the differentiation method B (FIG. 33B). TNNT2 expression in hepatocytes differentiating from pluripotent stem cells at various days using the differentiation C is shown in FIG. 33D.

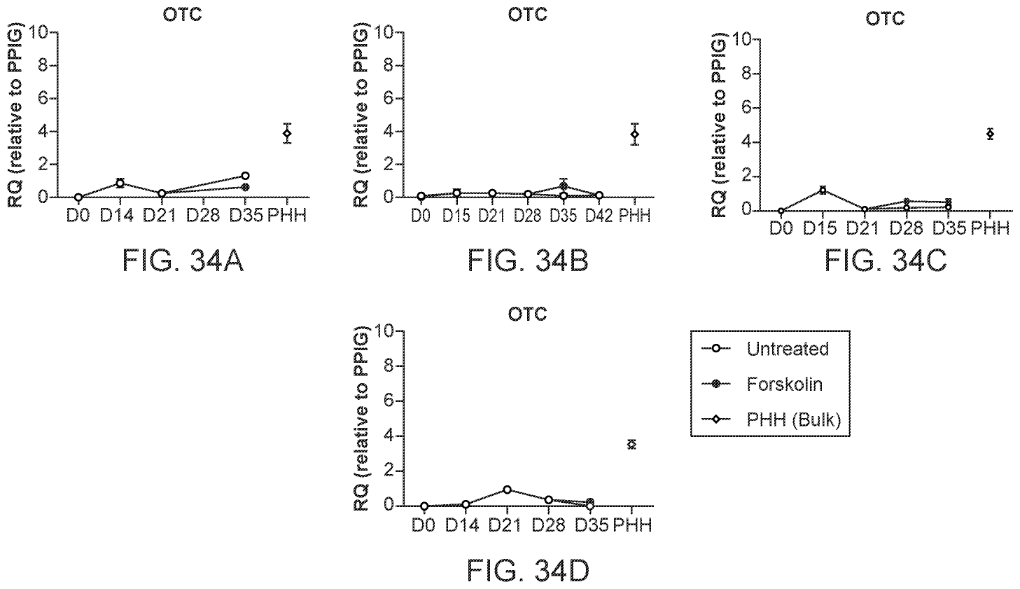

Differentiated hepatocytes expressed OTC, a urea cycle enzyme. FIGS. 34A-34D show OTC expression in hepatocytes differentiating from pluripotent stem cells at various days of the differentiation method A (FIGS. 34A and 34C), the differentiation method B (FIG. 34B), and the differentiation method C (FIG. 34D).

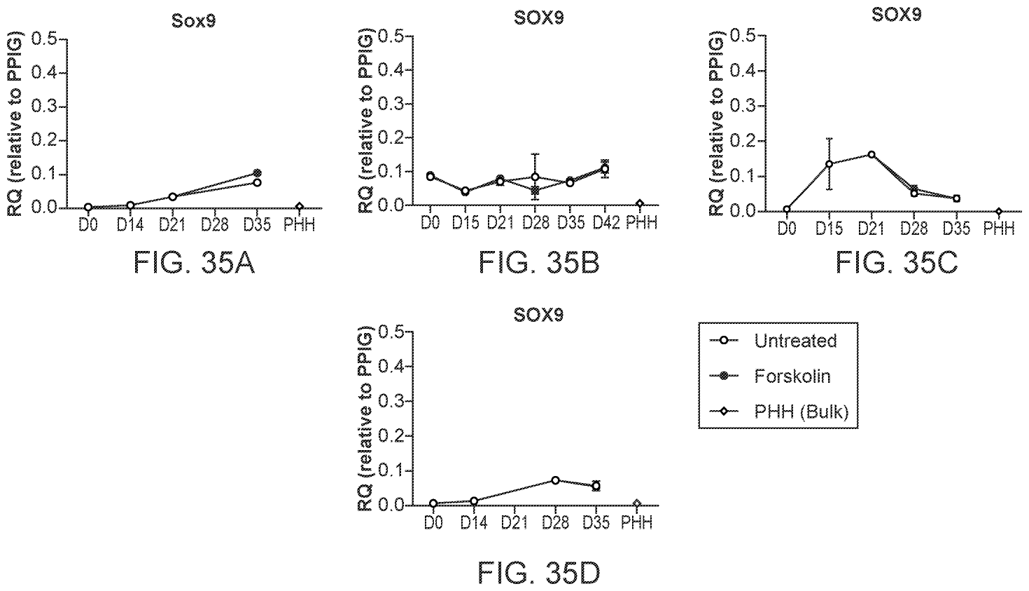

Differentiated hepatocytes expressed SOX9, a hepatocyte identity marker. FIGS. 35A-35D show SOX9 expression in hepatocytes differentiating from pluripotent stem cells at various days of the differentiation method A (FIGS. 35A and 35C), the differentiation method B (FIG. 35B) and the differentiation method C (FIG. 35D).

In this study, forskolin treatment was included at days 21-35 of the 3D hepatocyte differentiation process outlined above, at days 30-35 of the 3D hepatocyte differentiation process outlined above, or days 28-35 of the 2D hepatocyte differentiation process outlined above. Gene expression of numerous hepatocyte and urea cycle markers was measured at different time points. Forskolin addition during the maturation of the hepatocytes process increased expression of hepatocyte-specific genes and urea cycle enzyme genes including ALB, ARG1, ASGR1, ASL, CPS1, G6PC, HNF4a, and/or NAGS.

What is claimed is:

1. An isolated hepatocyte-like cell comprising enhanced ureagenesis capability.

2. The isolated hepatocyte-like cell of claim 1, wherein the cell produces converts ammonia at a rate of at least 3 nmol/min/10⁶ cells following stimulation with 5 mM or 10 mM ammonia.

3. The isolated hepatocyte-like cell of claim 1 or 2, wherein the cell has increased expression of one or more urea cycle pathway enzymes.

4. The isolated hepatocyte-like cell of any one of claims 1-3, wherein the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

5. The isolated hepatocyte-like cell of any one of claims 1-4, wherein the hepatocyte-like cell has increased RNA expression of the one or more urea cycle pathway enzymes.

6. The isolated hepatocyte-like cell of any one of claims 1-5, wherein the cell has increased protein expression of the one or more urea cycle pathway enzymes.

7. The isolated hepatocyte-like cell of any one of claims 1-6, wherein the cell has increased expression of one or more genes selected from the group consisting of albumin (ALB), asialoglycoprotein receptor 1 (ASGR1), ASGR2, alpha fetoprotein (AFP), glucose-6-phosphatase catalytic subunit (G6PC), hepatocyte nuclear factor 4 alpha (HNF4a), keratin, type I cytoskeletal 18 (KRT18), SOX9, SERPINA1, CYP1A1, CYP1A2, CYP2C19, CYP2B6, CYP2D6, CYP3A4, and CYP3A7.

8. The isolated hepatocyte-like cell of any one of claims 1-7, wherein the cell secretes one or more of albumin, α-1 antitrypsin (A1AT), and coagulation Factor V.

9. The isolated hepatocyte-like cell of any one of claims 1-7, wherein the cell has cytochrome p450 activity.

10. The isolated hepatocyte-like cell of claim 9, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

11. The isolated hepatocyte-like cell of any one of claims 1-7, wherein the cell has glycogen synthesis capability and/or storage capability.

12. The isolated hepatocyte-like cell of any one of claims 1-7, wherein the cell has low density lipoprotein (LDL) uptake and/or storage capability.

13. The isolated hepatocyte-like cell of any one of claims 1-7, wherein the cell has lipid storage capability.

14. The isolated hepatocyte-like cell of any one of claims 1-7, wherein the cell has indocyanine green (ICG) uptake and/or clearance capability.

15. The isolated hepatocyte-like cell of any one of claims 1-7, wherein the cell has gamma-glutamyl transpeptidase activity.

16. A composition comprising a population of the isolated hepatocyte-like cell of any one of claims 1-15.

17. The composition of claim 16, further comprising a pharmaceutically acceptable carrier.

18. A composition comprising:
a) a polymer matrix; and
b) a population of the isolated hepatocyte-like cell of any one of claims 1-15,
wherein the population of the isolated hepatocyte-like cell is encapsulated by the polymer matrix.

19. The composition of claim 18, wherein the polymer matrix is semipermeable.

20. The composition of claim 18, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

21. The composition of claim 18, wherein the population of the isolated hepatocyte-like cell comprise spheroids.

22. A method comprising:
a) providing a source cell;
b) differentiating the source cell in vitro in at least one culture medium comprising an agent that increases intracellular cyclic AMP, wherein the source cell differentiates into a mature hepatocyte-like cell having enhanced in vitro ureagenesis capability; and
c) recovering the mature hepatocyte-like cell.

23. The method of claim 22, wherein the source cell is a stem cell, a fibroblast, a gastric epithelial cell, a ductal cell, or a hepatocyte.

24. The method of claim 23, wherein the source cell is a stem cell.

25. The method of claim 24, wherein the stem cell is an induced pluripotent stem cell.

26. The method of any one of claims 22-25, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

27. The method of claim 26, wherein the agent is forskolin.

28. The method of claim 27, wherein the culture medium comprises 5-20 μM of forskolin.

29. The method of any one of claims 22-28, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

30. The method of any one of claims 22-29, wherein the source cell differentiates into a progenitor hepatocyte-like cell prior to differentiating into the mature hepatocyte-like cell.

31. The method of claim 30, wherein the differentiating step b) takes place after the source cell differentiates into the progenitor hepatocyte-like cell.

32. The method of any one of claims 22-29, wherein the source cell differentiates into an immature hepatocyte-like cell prior to differentiating into the mature hepatocyte-like cell.

33. The method of claim 32, wherein the differentiating step b) takes place during the differentiation of the immature hepatocyte-like cell to the mature hepatocyte-like cell.

34. The method of any one of claims 22-33, wherein the differentiating step b) takes places in a two-dimensional (2D) culture system.

35. The method of claim 34, wherein the two-dimensional culture system comprises a substrate comprising an extracellular matrix (ECM) component.

36. The method of claim 35, wherein the ECM component comprises laminin and/or collagen.

37. The method of claim 35, wherein the ECM component comprises an ECM maturation component.

38. The method of any one of claims 35-37, wherein the substrate is fetal bovine serum (FBS) free.

39. The method of claim 34, wherein the two-dimensional culture system comprises a soft hydrogel substrate.

40. The method of claim 39, wherein the soft hydrogel substrate has an elastic modulus ranging from about 1 kPa to about 8 kPa.

41. The method of claim 39 or 40, wherein the soft hydrogel substrate comprises poly(ethylene glycol) (PEG).

42. The method of any one of claims 22-33, wherein the differentiating step b) takes places in a three-dimensional culture system.

43. The method of claim 42, wherein the three-dimensional culture system comprises an inverse colloidal crystal scaffold.

44. The method of claim 43, wherein the inverse colloidal crystal scaffold is coated with an extracellular matrix (ECM) component.

45. The method of claim 44, wherein the extracellular matrix (ECM) component comprises laminin and/or collagen.

46. The method of any one of claims 22-45, wherein the differentiating step b) is carried out in the presence of an endothelial cell.

47. The method of claim 46, wherein the endothelial cell is a human umbilical vein endothelial cell (HUVEC).

48. The method of any one of claims 22-47, wherein the differentiating step b) is carried out at one or more oxygen conditions.

49. The method of claim 48, wherein the one or more oxygen conditions comprise a hypoxic condition.

50. The method of claim 48 or 49, wherein the one or more oxygen conditions comprise a normoxic condition.

51. The method of any one of claims 22-47, wherein the mature hepatocyte-like cell is contacted with vitamin K selected from the group consisting of vitamin K1, vitamin K2, and both vitamins K1 and K2.

52. The method of any one of claims 22-47, further comprising step d) encapsulating the mature hepatocyte-like cell in a polymer matrix.

53. The method of claim 52, wherein the polymer matrix is semipermeable.

54. The method of claim 53, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

55. The method of any one of claims 52-54, wherein the mature hepatocyte-like cell is cultured into a spheroid.

56. The method of any one of claims 29-55, wherein the mature hepatocyte-like cell has increased RNA expression of the one or more urea cycle pathway enzymes.

57. The method of any one of claims 29-56, wherein the mature hepatocyte-like cell has increased protein expression of the one or more urea cycle pathway enzymes.

58. The method of any one of claims 22-57, wherein the culture medium further comprises hepatocyte growth factor (HGF), and oncostatin-M (OSM).

59. The method of any one of claims 22-57, wherein the culture medium is free of hepatocyte growth factor (HGF).

60. The method of any one of claims 22-59, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

61. The method of any one of claims 22-60, wherein the mature hepatocyte-like cell secretes albumin and/or α-1 antitrypsin (A1AT).

62. The method of any one of claims 22-60, wherein the mature hepatocyte-like cell has cytochrome p450 activity.

63. The method of claim 62, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

64. The method of any one of claims 22-60, wherein the mature hepatocyte-like cell has glycogen synthesis capability and/or storage capability.

65. The method of any one of claims 22-60, wherein the mature hepatocyte-like cell has low density lipoprotein (LDL) uptake and/or storage capability.

66. The method of any one of claims 22-60, wherein the mature hepatocyte-like cell has lipid storage capability.

67. The method of any one of claims 22-60, wherein the mature hepatocyte-like cell has indocyanine green (ICG) uptake and/or clearance capability.

68. The method of any one of claims 22-60, wherein the mature hepatocyte-like cell has gamma-glutamyl transpeptidase activity.

69. A method for producing a mature hepatocyte-like cell having enhanced ureagenesis capability comprising:
a) providing a source cell;
b) differentiating the source cell in vitro in a two dimensional culture system comprising:
i. a fetal bovine serum (FBS) free substrate comprising an extracellular matrix (ECM) component; and
ii. at least one culture medium comprising an agent that increases intracellular cyclic AMP,
wherein the source cell differentiates into a mature hepatocyte-like cell having enhanced in vitro ureagenesis capability; and
c) recovering the mature hepatocyte-like cell.

70. The method of claim 69, wherein the extracellular matrix component comprises laminin and/or collagen.

71. The method of claim 69, wherein the source cell is a stem cell, a fibroblast, a gastric epithelial cell, a ductal cell, or a hepatocyte.

72. The method of claim 71, wherein the source cell is a stem cell.

73. The method of claim 72, wherein the stem cell is an induced pluripotent stem cell.

74. The method of any one of claims 69-73, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

75. The method of claim 74, wherein the agent is forskolin.

76. The method of claim 75, wherein the culture medium comprises 5-20 μM of forskolin.

77. The method of any one of claims 69-76, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

78. The method of any one of claims 69-77, wherein the source cell differentiates into a progenitor hepatocyte-like cell prior to differentiating into the mature hepatocyte-like cell.

79. The method of claim 78, wherein the differentiating step b) takes place after the source cell differentiates into the progenitor hepatocyte-like cell.

80. The method of any one of claims 69-77, wherein the source cell differentiates into an immature hepatocyte-like cell prior to differentiating into the mature hepatocyte-like cell.

81. The method of claim 80, wherein the differentiating step b) takes place during the differentiation of the immature hepatocyte-like cell to the mature hepatocyte-like cell.

82. The method of claim 79, wherein the differentiating step b) takes places in a two-dimensional (2D) culture system.

83. The method of claim 82, wherein the two-dimensional culture system comprises a substrate comprising an extracellular matrix (ECM) component.

84. The method of claim 83, wherein the ECM component comprises laminin and/or collagen.

85. The method of claim 83 or 84, wherein the ECM component comprises an ECM maturation component.

86. The method of claim 82, wherein the two-dimensional culture system comprises a soft hydrogel substrate.

87. The method of claim 86, wherein the soft hydrogel substrate has an elastic modulus ranging from about 1 kPa to about 8 kPa.

88. The method of claim 87, wherein the soft hydrogel substrate comprises poly(ethylene glycol) (PEG).

89. The method of any one of claims 69-81, wherein the differentiating step b) takes places in a three-dimensional culture system.

90. The method of claim 89, wherein the three-dimensional culture system comprises an inverse colloidal crystal scaffold.

91. The method of claim 90, wherein the inverse colloidal crystal scaffold is coated with an extracellular matrix (ECM) component.

92. The method of claim 91, wherein the extracellular matrix (ECM) component comprises laminin and/or collagen.

93. The method of any one of claims 69-92, wherein the differentiating step b) is carried out in the presence of an endothelial cell.

94. The method of claim 93, wherein the endothelial cell is a human umbilical vein endothelial cell (HUVEC).

95. The method of any one of claims 69-94, wherein the differentiating step b) is carried out at one or more oxygen conditions.

96. The method of claim 95, wherein the one or more oxygen conditions comprise a hypoxic condition.

97. The method of claim 95 or 96, wherein the one or more oxygen conditions comprise a normoxic condition.

98. The method of any one of claims 69-98, wherein the mature hepatocyte-like cell is contacted with vitamin K selected from the group consisting of vitamin K1, vitamin K2, and both vitamins K1 and K2.

99. The method of any one of claims 69-98, further comprising step d) encapsulating the mature hepatocyte-like cell in a polymer matrix.

100. The method of claim 99, wherein the polymer matrix is semipermeable.

101. The method of claim 100, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

102. The method of any one of claims 89-101, wherein the mature hepatocyte-like cell is cultured into a spheroid.

103. The method of any one of claims 77-102, wherein the mature hepatocyte-like cell has increased RNA expression of the one or more urea cycle pathway enzymes.

104. The method of any one of claims 77-103, wherein the mature hepatocyte-like cell has increased protein expression of the one or more urea cycle pathway enzymes.

105. The method of any one of claims 69-104, wherein the culture medium further comprises hepatocyte growth factor (HGF), and oncostatin-M (OSM).

106. The method of any one of claims 69-104, wherein the culture medium is free of hepatocyte growth factor (HGF).

107. The method of any one of claims 69-106, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

108. The method of any one of claims 69-106, wherein the mature hepatocyte-like cell secretes albumin and/or α-1 antitrypsin (A1AT).

109. The method of any one of claims 69-106, wherein the mature hepatocyte-like cell has cytochrome p450 activity.

110. The method of claim 109, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

111. The method of any one of claims 69-106, wherein the mature hepatocyte-like cell has glycogen synthesis capability and/or storage capability.

112. The method of any one of claims 69-106, wherein the mature hepatocyte-like cell has low density lipoprotein (LDL) uptake and/or storage capability.

113. The method of any one of claims 69-106, wherein the mature hepatocyte-like cell has lipid storage capability.

114. The method of any one of claims 69-106, wherein the mature hepatocyte-like cell has indocyanine green (ICG) uptake and/or clearance capability.

115. The method of any one of claims 69-106, wherein the mature hepatocyte-like cell has gamma-glutamyl transpeptidase activity.

116. A method for producing a mature hepatocyte-like cell having enhanced ureagenesis capability comprising:
a) providing a source cell;
b) differentiating the source cell in vitro in a two dimensional culture system comprising:
i. a soft hydrogel substrate; and
ii. at least one culture medium comprising an agent that increases intracellular cyclic AMP,
wherein the source cell differentiates into a mature hepatocyte-like cell having enhanced in vitro ureagenesis capability; and
c) recovering the mature hepatocyte-like cell.

117. The method of claim 116, wherein the soft hydrogel substrate has an elastic modulus ranging from about 1 kPa to about 8 kPa.

118. The method of claim 116 or 117, wherein the soft hydrogel substrate comprises poly(ethylene glycol) (PEG).

119. The method of any one of claims 116-118, wherein the source cell is a stem cell, a fibroblast, a gastric epithelial cell, a ductal cell, or a hepatocyte.

120. The method of claim 119, wherein the source cell is a stem cell.

121. The method of claim 120, wherein the stem cell is an induced pluripotent stem cell.

122. The method of any one of claims 116-121, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

123. The method of claim 122, wherein the agent is forskolin.

124. The method of claim 123, wherein the culture medium comprises 5-20 μM of forskolin.

125. The method of any one of claims 116-124, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

126. The method of any one of claims 116-125, wherein the source cell differentiates into a progenitor hepatocyte-like cell prior to differentiating into the mature hepatocyte-like cell.

127. The method of claim 126, wherein the differentiating step b) takes place after the source cell differentiates into the progenitor hepatocyte-like cell.

128. The method of any one of claims 116-125, wherein the source cell differentiates into an immature hepatocyte-like cell prior to differentiating into the mature hepatocyte-like cell.

129. The method of claim 128, wherein the differentiating step b) takes place during the differentiation of the immature hepatocyte-like cell to the mature hepatocyte-like cell.

130. The method of claim 127, wherein the differentiating step b) takes places in a two-dimensional (2D) culture system.

131. The method of any one of claims 116-130, wherein the two-dimensional culture system comprises a substrate comprising an extracellular matrix (ECM) component.

132. The method of claim 131, wherein the ECM component comprises laminin and/or collagen.

133. The method of claim 131 or 132, wherein the ECM component comprises an ECM maturation component.

134. The method of any one of claims 130-133, wherein the substrate is fetal bovine serum (FBS) free.

135. The method of claim 127, wherein the differentiating step b) takes places in a three-dimensional culture system.

136. The method of claim 135, wherein the three-dimensional culture system comprises an inverse colloidal crystal scaffold.

137. The method of claim 136, wherein the inverse colloidal crystal scaffold is coated with an extracellular matrix (ECM) component.

138. The method of claim 137, wherein the extracellular matrix (ECM) component comprises laminin and/or collagen.

139. The method of any one of claims 116-138, wherein the differentiating step b) is carried out in the presence of an endothelial cell.

140. The method of claim 139, wherein the endothelial cell is a human umbilical vein endothelial cell (HUVEC).

141. The method of any one of claims 116-140, wherein the differentiating step b) is carried out at one or more oxygen conditions.

142. The method of claim 141, wherein the one or more oxygen conditions comprise a hypoxic condition.

143. The method of claim 141 or 142, wherein the one or more oxygen conditions comprise a normoxic condition.

144. The method of any one of claims 116-143, wherein the mature hepatocyte-like cell is contacted with vitamin K selected from the group consisting of vitamin K1, vitamin K2, and both vitamins K1 and K2.

145. The method of any one of claims 116-144, further comprising step d) encapsulating the mature hepatocyte-like cell in a polymer matrix.

146. The method of claim 145, wherein the polymer matrix is semipermeable.

147. The method of claim 146, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

148. The method of any one of claims 145-147, wherein the mature hepatocyte-like cell is cultured into a spheroid.

149. The method of any one of claims 125-148, wherein the mature hepatocyte-like cell has increased RNA expression of the one or more urea cycle pathway enzymes.

150. The method of any one of claims 125-149, wherein the mature hepatocyte-like cell has increased protein expression of the one or more urea cycle pathway enzymes.

151. The method of any one of claims 116-150, wherein the culture medium further comprises hepatocyte growth factor (HGF), and oncostatin-M (OSM).

152. The method of any one of claims 116-150, wherein the culture medium is free of hepatocyte growth factor (HGF).

153. The method of any one of claims 116-152, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

154. The method of any one of claims 116-153, wherein the mature hepatocyte-like cell secretes albumin and/or α-1 antitrypsin (A1AT).

155. The method of any one of claims 116-153, wherein the mature hepatocyte-like cell has cytochrome p450 activity.

156. The method of claim 155, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

157. The method of any one of claims 116-153, wherein the mature hepatocyte-like cell has glycogen synthesis capability and/or storage capability.

158. The method of any one of claims 116-153, wherein the mature hepatocyte-like cell has low density lipoprotein (LDL) uptake and/or storage capability.

159. The method of any one of claims 116-153, wherein the mature hepatocyte-like cell has lipid storage capability.

160. The method of any one of claims 116-153, wherein the mature hepatocyte-like cell has indocyanine green (ICG) uptake and/or clearance capability.

161. The method of any one of claims 116-153, wherein the mature hepatocyte-like cell has gamma-glutamyl transpeptidase activity.

162. A method for producing a mature hepatocyte-like cell having enhanced ureagenesis capability comprising:
a) providing a source cell;
b) differentiating the source cell in vitro in at least one culture medium comprising an agent that increases intracellular cyclic AMP, wherein the source cell differentiates into a mature hepatocyte-like cell having enhanced in vitro ureagenesis capability; and
c) recovering the mature hepatocyte-like cell,
wherein the differentiating is initially carried out on a first substrate comprising gelatin and fetal bovine serum and transferred to a second substrate comprising laminin and/or collagen on about day 14 of the differentiating.

163. The method of claim 162, wherein the second substrate further comprises fetal bovine serum.

164. The method of claim 162 or 163, wherein the source cell is selected from the group consisting of a stem cell, a fibroblast, a gastric epithelial cell, a ductal cell, and a hepatocyte.

165. The method of claim 164, wherein the source cell is a stem cell.

166. The method of claim 165, wherein the stem cell is an induced pluripotent stem cell.

167. The method of any one of claims 162-166, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

168. The method of claim 167, wherein the agent is forskolin.

169. The method of claim 168, wherein the culture medium comprises 5-20 μM of forskolin.

170. The method of any one of claims 162-169, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

171. The method of any one of claims 162-170, wherein the source cell differentiates into a progenitor hepatocyte-like cell prior to differentiating into the mature hepatocyte-like cell.

172. The method of claim 171, wherein the differentiating step b) takes place after the source cell differentiates into the progenitor hepatocyte-like cell.

173. The method of any one of claims 162-170, wherein the source cell differentiates into an immature hepatocyte-like cell prior to differentiating into the mature hepatocyte-like cell.

174. The method of claim 173, wherein the differentiating step b) takes place during the differentiation of the immature hepatocyte-like cell to the mature hepatocyte-like cell.

175. The method of any one of claims 162-174, wherein the differentiating step b) is carried out in the presence of an endothelial cell.

176. The method of claim 175, wherein the endothelial cell is a human umbilical vein endothelial cell (HUVEC).

177. The method of any one of claims 162-176, wherein the differentiating step b) is carried out at one or more oxygen conditions.

178. The method of claim 176, wherein the one or more oxygen conditions comprise a hypoxic condition.

179. The method of claim 176 or 177, wherein the one or more oxygen conditions comprise a normoxic condition.

180. The method of any one of claims 162-179, wherein the mature hepatocyte-like cell is contacted with vitamin K selected from the group consisting of vitamin K1, vitamin K2, and both vitamins K1 and K2.

181. The method of any one of claims 162-180, further comprising step d) encapsulating the mature hepatocyte-like cell in a polymer matrix.

182. The method of claim 181, wherein the polymer matrix is semipermeable.

183. The method of claim 182, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

184. The method of any one of claims 181-183, wherein the mature hepatocyte-like cell is cultured into a spheroid.

185. The method of any one of claims 170-184, wherein the mature hepatocyte-like cell has increased RNA expression of the one or more urea cycle pathway enzymes.

186. The method of any one of claims 170-185, wherein the mature hepatocyte-like cell has increased protein expression of the one or more urea cycle pathway enzymes.

187. The method of any one of claims 162-186, wherein the culture medium further comprises hepatocyte growth factor (HGF), and oncostatin-M (OSM).

188. The method of any one of claims 162-186, wherein the culture medium is free of hepatocyte growth factor (HGF).

189. The method of any one of claims 162-188, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

190. The method of any one of claims 162-189, wherein the mature hepatocyte-like cell secretes albumin and/or α-1 antitrypsin (A1AT).

191. The method of any one of claims 162-189, wherein the mature hepatocyte-like cell has cytochrome p450 activity.

192. The method of claim 191, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

193. The method of any one of claims 162-189, wherein the mature hepatocyte-like cell has glycogen synthesis capability and/or storage capability.

194. The method of any one of claims 162-189, wherein the mature hepatocyte-like cell has low density lipoprotein (LDL) uptake and/or storage capability.

195. The method of any one of claims 162-189, wherein the mature hepatocyte-like cell has lipid storage capability.

196. The method of any one of claims 162-189, wherein the mature hepatocyte-like cell has indocyanine green (ICG) uptake and/or clearance capability.

197. The method of any one of claims 162-189, wherein the mature hepatocyte-like cell has gamma-glutamyl transpeptidase activity.

198. A method for producing a mature hepatocyte-like cell having enhanced ureagenesis capability comprising:
a) providing a source cell;
b) differentiating the source cell in vitro in a normoxic condition in at least one culture medium comprising an agent that increases intracellular cyclic AMP, wherein the source cell differentiates into a mature hepatocyte-like cell having enhanced in vitro ureagenesis capability; and
c) recovering the mature hepatocyte-like cell.

199. The method of claim 198, wherein the normoxic condition comprises about 20% partial pressure of 02.

200. The method of claim 198 or 199, wherein the source cell is selected from the group consisting of a stem cell, a fibroblast, a gastric epithelial cell, a ductal cell, and a hepatocyte.

201. The method of claim 200, wherein the source cell is a stem cell.

202. The method of claim 201, wherein the stem cell is an induced pluripotent stem cell.

203. The method of any one of claims 198-202, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

204. The method of claim 203, wherein the agent is forskolin.

205. The method of claim 204, wherein the culture medium comprises 5-20 μM of forskolin.

206. The method of any one of claims 198-205, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

207. The method of any one of claims 198-206, wherein the source cell differentiates into a progenitor hepatocyte-like cell prior to differentiating into the mature hepatocyte-like cell.

208. The method of claim 207, wherein the differentiating step b) takes place after the source cell differentiates into the progenitor hepatocyte-like cell.

209. The method of any one of claims 198-206, wherein the source cell differentiates into an immature hepatocyte-like cell prior to differentiating into the mature hepatocyte-like cell.

210. The method of claim 209, wherein the differentiating step b) takes place during the differentiation of the immature hepatocyte-like cell to the mature hepatocyte-like cell.

211. The method of claim 208, wherein the differentiating step b) takes places in a two-dimensional (2D) culture system.

212. The method of claim 211, wherein the two-dimensional culture system comprises a substrate comprising an extracellular matrix (ECM) component.

213. The method of claim 212, wherein the ECM component comprises laminin and/or collagen.

214. The method of claim 212 or 213, wherein the ECM component comprises an ECM maturation component.

215. The method of any one of claims 211-214, wherein the substrate is fetal bovine serum (FBS) free.

216. The method of claim 211, wherein the two-dimensional culture system comprises a soft hydrogel substrate.

217. The method of claim 216, wherein the soft hydrogel substrate has an elastic modulus ranging from about 1 kPa to about 8 kPa.

218. The method of claim 216 or 217, wherein the soft hydrogel substrate comprises poly(ethylene glycol) (PEG).

219. The method of any one of claims 208-210, wherein the differentiating step b) takes places in a three-dimensional culture system.

220. The method of claim 219, wherein the three-dimensional culture system comprises an inverse colloidal crystal scaffold.

221. The method of claim 220, wherein the inverse colloidal crystal scaffold is coated with an extracellular matrix (ECM) component.

222. The method of claim 221, wherein the extracellular matrix (ECM) component comprises laminin and/or collagen.

223. The method of any one of claims 198-222, wherein the differentiating step b) is carried out in the presence of an endothelial cell.

224. The method of claim 223, wherein the endothelial cell is a human umbilical vein endothelial cell (HUVEC).

225. The method of any one of claims 198-224, wherein the mature hepatocyte-like cell is contacted with vitamin K selected from the group consisting of vitamin K1, vitamin K2, and both vitamins K1 and K2.

226. The method of any one of claims 198-225, further comprising step d) encapsulating the mature hepatocyte-like cell in a polymer matrix.

227. The method of claim 226, wherein the polymer matrix is semipermeable.

228. The method of claim 227, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

229. The method of any one of claims 226-228, wherein the mature hepatocyte-like cell is cultured into a spheroid.

230. The method of any one of claims 206-229, wherein the mature hepatocyte-like cell has increased RNA expression of the one or more urea cycle pathway enzymes.

231. The method of any one of claims 206-230, wherein the mature hepatocyte-like cell has increased protein expression of the one or more urea cycle pathway enzymes.

232. The method of any one of claims 198-231, wherein the culture medium further comprises hepatocyte growth factor (HGF) and oncostatin-M (OSM).

233. The method of any one of claims 198-231, wherein the culture medium is free of hepatocyte growth factor (HGF).

234. The method of any one of claims 198-233, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

235. The method of any one of claims 198-233, wherein the mature hepatocyte-like cell secretes albumin and/or α-1 antitrypsin (A1AT).

236. The method of any one of claims 198-233, wherein the mature hepatocyte-like cell has cytochrome p450 activity.

237. The method of claim 236, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

238. The method of any one of claims 198-233, wherein the mature hepatocyte-like cell has glycogen synthesis capability and/or storage capability.

239. The method of any one of claims 198-233, wherein the mature hepatocyte-like cell has low density lipoprotein (LDL) uptake and/or storage capability.

240. The method of any one of claims 198-233, wherein the mature hepatocyte-like cell has lipid storage capability.

241. The method of any one of claims 198-233, wherein the mature hepatocyte-like cell has indocyanine green (ICG) uptake and/or clearance capability.

242. The method of any one of claims 198-233, wherein the mature hepatocyte-like cell has gamma-glutamyl transpeptidase activity.

243. A method for producing a mature hepatocyte-like cell having enhanced ureagenesis capability comprising:
a) providing a source cell;
b) differentiating the source cell in vitro in at least one culture medium comprising vitamin K1, wherein the source cell differentiates into a mature hepatocyte-like cell having enhanced in vitro ureagenesis capability; and
c) recovering the mature hepatocyte-like cell.

244. The method of claim 243, wherein the vitamin K1 is at a concentration of 750 μM-10 mM.

245. The method of claim 243 or 244, wherein the source cell is selected from the group consisting of a stem cell, a fibroblast, a gastric epithelial cell, a ductal cell, and a hepatocyte.

246. The method of claim 245, wherein the source cell is a stem cell.

247. The method of claim 246, wherein the stem cell is an induced pluripotent stem cell.

248. The method of any one of claims 243-247, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

249. The method of claim 248, wherein the agent is forskolin.

250. The method of claim 249, wherein the culture medium comprises 5-20 μM of forskolin.

251. The method of any one of claims 243-250, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

252. The method of any one of claims 243-251, wherein the source cell differentiates into a progenitor hepatocyte-like cell prior to differentiating into the mature hepatocyte-like cell.

253. The method of claim 252, wherein the differentiating step b) takes place after the source cell differentiates into the progenitor hepatocyte-like cell.

254. The method of any one of claims 243-251, wherein the source cell differentiates into an immature hepatocyte-like cell prior to differentiating into the mature hepatocyte-like cell.

255. The method of claim 254, wherein the differentiating step b) takes place during the differentiation of the immature hepatocyte-like cell to the mature hepatocyte-like cell.

256. The method of claim 253, wherein the differentiating step b) takes places in a two-dimensional (2D) culture system.

257. The method of claim 256, wherein the two-dimensional culture system comprises a substrate comprising an extracellular matrix (ECM) component.

258. The method of claim 257, wherein the ECM component comprises laminin and/or collagen.

259. The method of claim 257 or 258, wherein the ECM component comprises an ECM maturation component.

260. The method of any one of claims 256-259, wherein the substrate is fetal bovine serum (FBS) free.

261. The method of claim 256, wherein the two-dimensional culture system comprises a soft hydrogel substrate.

262. The method of claim 261, wherein the soft hydrogel substrate has an elastic modulus ranging from about 1 kPa to about 8 kPa.

263. The method of claim 261 or 262, wherein the soft hydrogel substrate comprises poly(ethylene glycol) (PEG).

264. The method of claim 253, wherein the differentiating step b) takes places in a three-dimensional culture system.

265. The method of claim 264, wherein the three-dimensional culture system comprises an inverse colloidal crystal scaffold.

266. The method of claim 265, wherein the inverse colloidal crystal scaffold is coated with an extracellular matrix (ECM) component.

267. The method of claim 266, wherein the extracellular matrix (ECM) component comprises laminin and/or collagen.

268. The method of any one of claims 243-267, wherein the differentiating step b) is carried out in the presence of an endothelial cell.

269. The method of claim 268, wherein the endothelial cell is a human umbilical vein endothelial cell (HUVEC).

270. The method of any one of claims 243-269, wherein the differentiating step b) is carried out at one or more oxygen conditions.

271. The method of claim 270, wherein the one or more oxygen conditions comprise a hypoxic condition.

272. The method of claim 270 or 271, wherein the one or more oxygen conditions comprise a normoxic condition.

273. The method of any one of claims 243-272, further comprising step d) encapsulating the mature hepatocyte-like cell in a polymer matrix.

274. The method of claim 273, wherein the polymer matrix is semipermeable.

275. The method of claim 274, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and combination thereof.

276. The method of any one of claims 273-275, wherein the mature hepatocyte-like cell is cultured into a spheroid.

277. The method of any one of claims 251-276, wherein the mature hepatocyte-like cell has increased RNA expression of the one or more urea cycle pathway enzymes.

278. The method of any one of claims 251-277, wherein the mature hepatocyte-like cell has increased protein expression of the one or more urea cycle pathway enzymes.

279. The method of any one of claims 243-278, wherein the culture medium further comprises hepatocyte growth factor (HGF), and oncostatin-M (OSM).

280. The method of any one of claims 243-278, wherein the culture medium is free of hepatocyte growth factor (HGF).

281. The method of any one of claims 243-280, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

282. The method of any one of claims 243-281, wherein the mature hepatocyte-like cell secretes albumin and/or α-1 antitrypsin (A1AT).

283. The method of any one of claims 243-281, wherein the mature hepatocyte-like cell has cytochrome p450 activity.

284. The method of claim 283, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

285. The method of any one of claims 243-281, wherein the mature hepatocyte-like cell has glycogen synthesis capability and/or storage capability.

286. The method of any one of claims 243-281, wherein the mature hepatocyte-like cell has low density lipoprotein (LDL) uptake and/or storage capability.

287. The method of any one of claims 243-281, wherein the mature hepatocyte-like cell has lipid storage capability.

288. The method of any one of claims 243-281, wherein the mature hepatocyte-like cell has indocyanine green (ICG) uptake and/or clearance capability.

289. The method of any one of claims 243-281, wherein the mature hepatocyte-like cell has gamma-glutamyl transpeptidase activity.

290. A method for treating a liver disorder in a patient comprising administering to the patient a therapeutically effective amount of a population of hepatocyte-like cells comprising enhanced ureagenesis capability.

291. The method of claim 290, wherein the liver disorder is selected from the group consisting of fibrosis, cirrhosis, end-stage liver disease, a metabolic liver disease, acute liver failure, and chronic liver failure.

292. The method of claim 290 or 291, wherein the administering comprises grafting the population of hepatocyte-like cells into the patient's liver.

293. The method of claim 292, wherein the grafting comprises injecting the population of hepatocyte-like cells into the patient.

294. The method of claim 293, wherein the injecting of the population of hepatocyte-like cells is into the patient's liver.

295. The method of any one of claims 290-294, wherein the population of hepatocyte-like cells converts ammonia at a rate of at least 3 nmol/min/10⁶ cells following stimulation with 5 mM or 10 mM ammonia.

296. The method of any one of claims 290-295, wherein the hepatocyte-like cells further have increased expression of one or more urea cycle pathway enzymes.

297. The method of claim 296, wherein the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

298. The method of any one of claims 290-297, wherein the hepatocyte-like cells have increased RNA expression of the one or more urea cycle pathway enzymes.

299. The method of any one of claims 290-298, wherein the hepatocyte-like cells has increased protein expression of the one or more urea cycle pathway enzymes.

300. The method of any one of claims 290-299, wherein the hepatocyte-like cells have increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

301. The method of any one of claims 290-300, wherein the hepatocyte-like cells secrete albumin and/or α-1 antitrypsin (A1AT).

302. The method of any one of claims 290-300, wherein the hepatocyte-like cells have cytochrome p450 activity.

303. The method of claim 302, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

304. The method of any one of claims 290-300, wherein the hepatocyte-like cells have glycogen synthesis capability and/or storage capability.

305. The method of any one of claims 290-300, wherein the hepatocyte-like cells have low density lipoprotein (LDL) uptake and/or storage capability.

306. The method of any one of claims 290-300, wherein the hepatocyte-like cells have lipid storage capability.

307. The method of any one of claims 290-300, wherein the hepatocyte-like cells have indocyanine green (ICG) uptake and/or clearance capability.

308. The method of any one of claims 290-300, wherein the hepatocyte-like cells have gamma-glutamyl transpeptidase activity.

309. A mature hepatocyte-like cell differentiated from a source cell, wherein the mature hepatocyte-like cell has increased ureagenesis capability, wherein the source cell is differentiated in vitro in at least one culture medium comprising an agent that increases intracellular cyclic AMP.

310. The mature hepatocyte-like cell of claim 309, wherein the source cell is selected from the group consisting of a stem cell, a fibroblast, a gastric epithelial cell, a ductal cell, and a hepatocyte.

311. The mature hepatocyte-like cell of claim 310, wherein the source cell is a stem cell.

312. The mature hepatocyte-like cell of claim 311, wherein the stem cell is an induced pluripotent stem cell.

313. The mature hepatocyte-like cell of any one of claims 309-312, wherein the agent is selected from the group consisting of forskolin, glucagon, glucagon-like peptide-1 (GLP-1), glucose-dependent insulinotropic peptide (GIP), and an analog thereof.

314. The mature hepatocyte-like cell of claim 313, wherein the agent is forskolin.

315. The mature hepatocyte-like cell of claim 314, wherein the culture medium comprises 5-20 μM of forskolin.

316. The mature hepatocyte-like cell of any one of claims 309-315, wherein the agent increases the expression of one or more urea cycle pathway enzymes selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

317. The mature hepatocyte-like cell of any one of claims 309-316, wherein the mature hepatocyte-like cell has increased RNA expression of the one or more urea cycle pathway enzymes.

318. The mature hepatocyte-like cell of any one of claims 309-317, wherein the mature hepatocyte-like cell has increased protein expression of the one or more urea cycle pathway enzymes.

319. The mature hepatocyte-like cell of any one of claims 309-318, wherein the source cell differentiates into a progenitor hepatocyte-like cell prior to differentiating into the mature hepatocyte-like cell.

320. The mature hepatocyte-like cell of claim 319, wherein the differentiating step b) takes place after the source cell differentiates into the progenitor hepatocyte-like cell.

321. The mature hepatocyte-like cell of any one of claims 309-320, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

322. The mature hepatocyte-like cell of any one of claims 309-321, wherein the mature hepatocyte-like cell secretes albumin and/or α-1 antitrypsin (A1AT).

323. The mature hepatocyte-like cell of any one of claims 309-321, wherein the mature hepatocyte-like cell has cytochrome p450 activity.

324. The method of claim 323, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

325. The mature hepatocyte-like cell of any one of claims 309-321, wherein the mature hepatocyte-like cell has glycogen synthesis capability and/or storage capability.

326. The mature hepatocyte-like cell of any one of claims 309-321, wherein the mature hepatocyte-like cell has low density lipoprotein (LDL) uptake and/or storage capability.

327. The mature hepatocyte-like cell of any one of claims 309-321, wherein the mature hepatocyte-like cell has lipid storage capability.

328. The mature hepatocyte-like cell of any one of claims 309-321, wherein the mature hepatocyte-like cell has indocyanine green (ICG) uptake and/or clearance capability.

329. The mature hepatocyte-like cell of any one of claims 309-321, wherein the mature hepatocyte-like cell has gamma-glutamyl transpeptidase activity.

330. A composition comprising:
a) a polymer matrix; and
b) a population of the mature hepatocyte-like cell of any one of claims 309-329, wherein the population of the isolated hepatocyte-like cell is encapsulated by the polymer matrix.

331. The composition of claim 330, wherein the polymer matrix is semipermeable.

332. The composition of claim 331, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

333. The composition of any one of claims 330-332, wherein population of the mature hepatocyte-like cell comprises spheroids.

334. A method comprising
a) providing a mature hepatocyte-like cell that has been differentiated in vitro from a source cell that is not a hepatocyte or hepatocyte-like cell;
b) culturing the mature hepatocyte-like cell in at least one culture medium comprising an agent that increases intracellular cyclic AMP; and
c) recovering the mature hepatocyte-like cell.

335. The method of claim 334, wherein the source cell is selected from the group consisting of a stem cell, a fibroblast, a gastric epithelial cell, and a ductal cell.

336. The method of claim 335, wherein the source cell is a stem cell.

337. The method of claim 336, wherein the stem cell is an induced pluripotent stem cell.

338. The method of any one of claims 334-337, wherein the agent is selected from the group consisting of forskolin, glucagon, glucagon-like peptide-1 (GLP-1), glucose-dependent insulinotropic peptide (GIP), a phosphodiesterase inhibitor, and an analog of any of the foregoing.

339. The method of claim 338, wherein the agent is forskolin.

340. The method of claim 339, wherein the culture medium comprises 5-20 μM of forskolin.

341. A hepatocyte-like cell comprising enhanced ureagenesis capability for the treatment of a liver disorder.

342. The hepatocyte-like cell of claim 341, wherein the liver disorder is selected from fibrosis, cirrhosis, end-stage liver disease, a metabolic liver disease, acute liver failure, and chronic liver failure.

343. The hepatocyte-like cell of claim 341 or 342, wherein the cell converts ammonia at a rate of at least 3 nmol/min/10⁶ cells following stimulation with 5 mM or 10 mM ammonia.

344. The hepatocyte-like cell of any one of claims 341-343, wherein the cell further has increased expression of one or more urea cycle pathway enzymes.

345. The hepatocyte-like cell of claim 344, wherein the one or more urea cycle pathway enzymes are selected from the group consisting of forskolin carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

346. The hepatocyte-like cell of any one of claims 341-345, wherein the cell has increased RNA expression of the one or more urea cycle pathway enzymes.

347. The hepatocyte-like cell of any one of claims 341-346, wherein the cell has increased protein expression of the one or more urea cycle pathway enzymes.

348. The hepatocyte-like cell of any one of claims 341-347, wherein the cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

349. The hepatocyte-like cell of any one of claims 341-348, wherein the cell secretes albumin and/or α-1 antitrypsin (A1AT).

350. The hepatocyte-like cell of any one of claims 341-348, wherein the cell has cytochrome p450 activity.

351. The hepatocyte-like cell of claim 350, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

352. The hepatocyte-like cell of any one of claims 341-348, wherein the cell has glycogen synthesis capability and/or storage capability.

353. The hepatocyte-like cell of any one of claims 341-348, wherein the cell has low density lipoprotein (LDL) uptake and/or storage capability.

354. The hepatocyte-like cell of any one of claims 341-348, wherein the cell has lipid storage capability.

355. The hepatocyte-like cell of any one of claims 341-348, wherein the cell has indocyanine green (ICG) uptake and/or clearance capability.

356. Use of a hepatocyte-like cell comprising enhanced ureagenesis capability in the manufacture of a medicament for the treatment of a liver disorder in a patient in need thereof.

357. The use of claim 356, wherein the liver disorder is selected from the group consisting of fibrosis, cirrhosis, end-stage liver disease, a metabolic liver disease, acute liver failure, and chronic liver failure.

358. The use of claim 356 or 357, wherein the hepatocyte-like cell converts ammonia at a rate of at least 3 nmol/min/10⁶ cells following stimulation with 5 mM or 10 mM ammonia.

359. The use of any one of claims 356-358, wherein the hepatocyte-like cell further has increased expression of one or more urea cycle pathway enzymes.

360. The use of claim 359, wherein the one or more urea cycle pathway enzymes are selected from the group consisting of carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

361. The use of any one of claims 356-360, wherein the hepatocyte-like cell has increased RNA expression of the one or more urea cycle pathway enzymes.

362. The use of any one of claims 356-361, wherein the hepatocyte-like cell has increased protein expression of the one or more urea cycle pathway enzymes.

363. The use of any one of claims 356-362, wherein the hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

364. The use of any one of claims 356-363, wherein the hepatocyte-like cell secretes albumin and/or α-1 antitrypsin (A1AT).

365. The use of any one of claims 356-363, wherein the hepatocyte-like cell has cytochrome p450 activity.

366. The use of claim 365, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

367. The use of any one of claims 356-363, wherein the hepatocyte-like cell has glycogen synthesis capability and/or storage capability.

368. The use of any one of claims 356-363, wherein the hepatocyte-like cell has low density lipoprotein (LDL) uptake and/or storage capability.

369. The use of any one of claims 356-363, wherein the hepatocyte-like cell has lipid storage capability.

370. The use of any one of claims 356-363, wherein the hepatocyte-like cell has indocyanine green (ICG) uptake and/or clearance capability.

371. The use of any one of claims 356-363, wherein the hepatocyte-like cell has gamma-glutamyl transpeptidase activity.

372. A method for producing a mature hepatocyte-like cell having enhanced ureagenesis capability comprising:
a) providing a source cell;
b) differentiating the source cell in vitro in a three dimensional culture system comprising at least one culture medium comprising an agent that increases intracellular cyclic AMP,
wherein the source cell differentiates into a mature hepatocyte-like cell having enhanced in vitro ureagenesis capability; and
c) recovering the mature hepatocyte-like cell.

373. The method of claim 372, wherein the three dimensional culture system further comprises a soft hydrogel substrate.

374. The method of claim 373, wherein the soft hydrogel substrate has an elastic modulus ranging from about 1 kPa to about 8 kPa.

375. The method of claim 373 or 374, wherein the soft hydrogel substrate comprises poly(ethylene glycol) (PEG).

376. The method of claim 372, wherein the three-dimensional culture system comprises an inverse colloidal crystal scaffold.

377. The method of claim 376, wherein the inverse colloidal crystal scaffold is coated with an extracellular matrix (ECM) component.

378. The method of claim 377, wherein the extracellular matrix (ECM) component comprises laminin and/or collagen.

379. The method of any one of claims 372-378, wherein the three dimensional culture system further comprises a polymer matrix.

380. The method of claim 379, wherein the polymer matrix is semipermeable.

381. The method of claim 379 or 380, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

382. The method of any one of claims 372-373, wherein the source cell is selected from the group consisting of a stem cell, a fibroblast, a gastric epithelial cell, and a ductal cell.

383. The method of claim 382, wherein the source cell is a stem cell.

384. The method of claim 383, wherein the stem cell is an induced pluripotent stem cell.

385. The method any one of claims 372-384, wherein the agent is selected from the group consisting of forskolin, glucagon, glucagon-like peptide-1 (GLP-1), glucose-dependent insulinotropic peptide (GIP), and an analog thereof.

386. The method of claim 385, wherein the agent is forskolin.

387. The method of claim 386, wherein the culture medium comprises 5-20 μM of forskolin.

388. The method of any one of claims 372-387, wherein the culture medium further comprises hepatocyte growth factor (HGF) and oncostatin-M (OSM).

389. The method of any one of claims 372-387, wherein the culture medium is free of hepatocyte growth factor (HGF).

390. The method of any one of claims 372-387, wherein the differentiating step b) is carried out at one or more oxygen conditions.

391. The method of claim 390, wherein the one or more oxygen conditions comprise a hypoxic condition.

392. The method of claim 387 or 390, wherein the one or more oxygen conditions comprise a normoxic condition.

393. The method of any one of claims 372-392, wherein the mature hepatocyte-like cell is contacted with vitamin K selected from the group consisting of vitamin K1, vitamin K2, and both vitamins K1 and K2.

394. The method of any one of claims 372-393, wherein the mature hepatocyte-like cell further has increased expression of one or more urea cycle pathway enzymes.

395. The method of claim 394, wherein the one or more urea cycle pathway enzymes are selected from the group consisting of forskolin carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

396. The method of any one of claims 372-395, wherein the mature hepatocyte-like cell has increased RNA expression of the one or more urea cycle pathway enzymes.

397. The method of any one of claims 372-396, wherein the mature hepatocyte-like cell has increased protein expression of the one or more urea cycle pathway enzymes.

398. The method of any one of claims 372-397, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

399. The method of any one of claims 372-398, wherein the mature hepatocyte-like cell secretes albumin and/or α-1 antitrypsin (A1AT).

400. The method of any one of claims 372-398, wherein the mature hepatocyte-like cell has cytochrome p450 activity.

401. The method of claim 400, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

402. The method of any one of claims 372-398, wherein the mature hepatocyte-like cell has glycogen synthesis capability and/or storage capability.

403. The method of any one of claims 372-398, wherein the mature hepatocyte-like cell has low density lipoprotein (LDL) uptake and/or storage capability.

404. The method of any one of claims 372-398, wherein the mature hepatocyte-like cell has lipid storage capability.

405. The method of any one of claims 372-398, wherein the mature hepatocyte-like cell has indocyanine green (ICG) uptake and/or clearance capability.

406. A method for producing a mature hepatocyte-like cell having enhanced ureagenesis capability comprising:
a) providing a source cell;
b) differentiating the source cell in vitro in a three dimensional culture system comprising:
i. a fetal bovine serum (FBS) free substrate; and
ii. at least one culture medium comprising an agent that increases intracellular cyclic AMP,
wherein the source cell differentiates into a mature hepatocyte-like cell having enhanced in vitro ureagenesis capability; and
c) recovering the mature hepatocyte-like cell.

407. The method of claim 406, wherein the FBS free substrate comprises a soft hydrogel substrate.

408. The method of claim 407, wherein the soft hydrogel substrate has an elastic modulus ranging from about 1 kPa to about 8 kPa.

409. The method of claim 407 or 408, wherein the soft hydrogel substrate comprises poly(ethylene glycol) (PEG).

410. The method of claim 406 or 407, wherein the three-dimensional culture system comprises an inverse colloidal crystal scaffold.

411. The method of claim 410, wherein the inverse colloidal crystal scaffold is coated with an extracellular matrix (ECM) component.

412. The method of claim 411, wherein the extracellular matrix (ECM) component comprises laminin and/or collagen.

413. The method of any one of claims 406-409, wherein the three dimensional culture system further comprises a polymer matrix.

414. The method of claim 413, wherein the polymer matrix is semipermeable.

415. The method of claim 413 or 414, wherein the polymer matrix comprises a matrix component selected from the group consisting of alginate, a modified alginate, chitosan, hydroxyethyl methacrylate, methyl methacrylate, agarose, collagen, polylysine, polyethersulfone, polysulfone, polyornithine, aminopropylsilicate, and any combination thereof.

416. The method of any one of claims 406-415, wherein the source cell is selected from the group consisting of a stem cell, a fibroblast, a gastric epithelial cell, and a ductal cell.

417. The method of claim 416, wherein the source cell is a stem cell.

418. The method of claim 417, wherein the stem cell is an induced pluripotent stem cell.

419. The method any one of claims 406-418, wherein the agent is selected from the group consisting of forskolin, glucagon, glucagon-like peptide-1 (GLP-1), glucose-dependent insulinotropic peptide (GIP), and an analog thereof.

420. The method of claim 419, wherein the agent is forskolin.

421. The method of claim 420, wherein the culture medium comprises 5-20 μM of forskolin.

422. The method of any one of claims 406-421, wherein the culture medium further comprises hepatocyte growth factor (HGF) and oncostatin-M (OSM).

423. The method of any one of claims 406-421, wherein the culture medium is free of hepatocyte growth factor (HGF).

424. The method of any one of claims 406-423, wherein the differentiating step b) is carried out at one or more oxygen conditions.

425. The method of claim 424, wherein the one or more oxygen conditions comprise a hypoxic condition.

426. The method of claim 424 or 425, wherein the one or more oxygen conditions comprise a normoxic condition.

427. The method of any one of claims 406-426, wherein the mature hepatocyte-like cell is contacted with vitamin K selected from the group consisting of vitamin K1, vitamin K2, and both vitamins K1 and K2.

428. The method of any one of claims 406-427, wherein the mature hepatocyte-like cell further has increased expression of one or more urea cycle pathway enzymes.

429. The method of claim 428, wherein the one or more urea cycle pathway enzymes are selected from the group consisting of forskolin carbamoylphosphate synthetase I (CPS1), ornithine transcarbamylase (OTC), argininosuccinic acid synthetase (ASS1), argininosuccinic acid lyase (ASL), arginase (ARG1), N-acetyl glutamate synthetase (NAGS), ornithine translocase (ORNT1), and citrin.

430. The method of any one of claims 406-429, wherein the mature hepatocyte-like cell has increased RNA expression of the one or more urea cycle pathway enzymes.

431. The method of any one of claims 406-430, wherein the mature hepatocyte-like cell has increased protein expression of the one or more urea cycle pathway enzymes.

432. The method of any one of claims 406-431, wherein the mature hepatocyte-like cell has increased expression of one or more genes selected from the group consisting of ALB, ASGR1, ASGR2, AFP, G6PC, HNF4a, KRT18, SOX9, SERPINA1, CYP1A1, CYP1A2, CYP2C19, CYP2B6, CYP2D6, CYP3A4, and CYP3A7.

433. The method of any one of claims 406-432, wherein the mature hepatocyte-like cell secretes albumin and/or α-1 antitrypsin (A1AT).

434. The method of any one of claims 406-432, wherein the mature hepatocyte-like cell has cytochrome p450 activity.

435. The method of claim 434, wherein the cytochrome p450 activity comprises activity of one or more cytochrome p450 family members selected from the group consisting of CYP1A1, CYP1A2, CYP2C19, CYP2B6, CYP2D6, CYP3A4, and CYP3A7.

436. The method of any one of claims 406-432, wherein the mature hepatocyte-like cell has glycogen synthesis capability and/or storage capability.

437. The method of any one of claims 406-432, wherein the mature hepatocyte-like cell has low density lipoprotein (LDL) uptake and/or storage capability.

438. The method of any one of claims 406-432, wherein the mature hepatocyte-like cell has lipid storage capability.

439. The method of any one of claims 406-432, wherein the mature hepatocyte-like cell has indocyanine green (ICG) uptake and/or clearance capability.
